(19) United States
(12) Patent Application Publication
(10) Pub. No.: US 2025/0154279 A1
(43) Pub. Date: May 15, 2025

(54) ANTI-GPC3 AND ANTI-CD137 MULTISPECIFIC ANTIBODIES AND METHODS OF USE

(71) Applicant: BeiGene, Ltd., Grand Cayman (KY)

(72) Inventors: Liang QU, Beijing (CN); Liu XUE, Beijing (CN); Ruyue JI, Shanghai (CN); Xi YUAN, Beijing (CN); Zhuo LI, Shanghai (CN); Jie LI, Shanghai (CN); Jian SUN, Beijing (CN)

(73) Assignee: BeiGene, Ltd., Grand Cayman (KY)

(21) Appl. No.: 18/954,427

(22) Filed: Nov. 20, 2024

Related U.S. Application Data
(63) Continuation of application No. PCT/CN2023/132564, filed on Nov. 20, 2023.

(30) Foreign Application Priority Data
Nov. 21, 2022 (WO) PCT/CN2022/133147
Nov. 21, 2022 (WO) PCT/CN2022/133148
Oct. 24, 2023 (WO) PCT/CN2023/126252

(51) Int. Cl. C07K 16/30; A61K 39/00; A61P 35/00; C07K 16/28

(52) U.S. Cl. CPC C07K 16/303; A61P 35/00; C07K 16/2878; A61K 2039/505; C07K 2317/31; C07K 2317/52; C07K 2317/565

(57) ABSTRACT

The present disclosure provides for the antibodies or antigen-binding fragment thereof that specifically binds human CD137, multispecific antibodies and antigen-binding fragments thereof that specifically bind to human GPC3 and CD137, a pharmaceutical composition comprising said antibody, and use of the antibody, multispecific antibody or the composition for treating a disease, such as cancer.

### CROSS-REFERENCE TO RELATED APPLICATIONS

This application is a bypass continuation of International Patent Application No. PCT/CN2023/132564, filed Nov. 20, 2023, which claims priority from International Patent Application Nos. PCT/CN2022/133148, filed Nov. 21, 2022, PCT/CN2022/133147, filed Nov. 21, 2022, and PCT/CN2023/126252, filed Oct. 24, 2023. All of these applications are incorporated herein by reference in their entirety.

### SEQUENCE LISTING

The contents of the electronic sequence listing submitted herewith named 138881_1048_Sequence_Listing_xml, was created on Nov. 20, 2023, is 130 bytes in size, and is incorporated herein by reference in its entirety.

### FIELD OF THE DISCLOSURE

Disclosed herein are antibodies that specifically bind to human CD137 (TNF receptor superfamily member 9 (TNFRSF9)), multispecific antibodies or antigen-binding fragments thereof that bind to human CD137, a composition comprising said antibodies, as well as methods or use for the treatment of cancer.

### BACKGROUND

Glypican-3 (GPC3) belongs to the heparan sulfate proteoglycan (HSPG) family, including a 60-70 kD core protein, which is linked to the surface of the cell membrane by a glycosylphosphatidylinositol anchor (GPI). The carboxy terminus of GPC3 is modified by a heparan sulfate side chain (Filmus J et al., J. Clin. Inv. 2001; 108: 497-501).

The specific expression of GPC3 in tumor cells has received widespread attention. GPC3 is expressed in hepatocellular carcinoma (HCC), the most common type of liver cancer. Notably, its expression is not detected in non-malignant tissues. The overexpression of GPC3 has also been reported in hepatoblastoma, lung squamous cell carcinoma (LSCC), and other cancers. Accordingly, GPC3 is suitable for targeted therapy as a tumor antigen. (Li N et al., Trends Cancer. 2018; 4: 741-54; Ho M, et al., Eur J Cancer. 2011; 47: 333-8; Moek et al., Am. J. Pathol. 2018; 188 (9): 1973-1981).

CD137 (also known as TNFRSF9/41BB) is a co-stimulatory molecule belonging to the TNFRSF family. It was discovered by T-cell-factor-screening on mouse helper and cytotoxic cells stimulated by concanavalin A. It was identified in 1989 as an inducible gene that was expressed on antigen-primed T cells but not on resting ones (Kwon et al., Proc. Natl. Acad. Sci. USA. 1989; 86:1963-1967). In addition, it is known to be expressed in dendritic cells (DCs), natural killer cells (NKs) (Vinay et al., Mol. Cancer Ther. 2012; 11:1062-1070), activated CD4+ and CD8+T lymphocytes, eosinophils, natural killer T cells (NKTs), and mast cells (Kwon et al., 1989 supra; Vinay D., Int. J. Hematol. 2006; 83:23-28). CD137 sustains and augments immune effector functions by inducing ThI cytokine production (Bartkowiak et al., Front Oncol. 2015; 5:117; Shuford et al., J Exp Med. 1997; 186:47-55). Upon binding to its sole ligand (CD137L, 4-1BBL, or TNFSF9), CD137 signaling results in increased expression of pro-survival molecules via NF-κB pathway activation (Wang et al., Immunol Rev. 2009; 229:192-215).

The anti-CD137 antibodies Urelumab (BMS-663513) which binds to CRD I of CD137 and Utomilumab (PF-05082566) which binds to CRDs III and IV of CD137 show potential as cancer therapeutics for their ability to activate cytotoxic T cells and to increase the production of interferon gamma (IFN-7). The mechanisms underlying tumor regression by these antibodies are the augmentive effects they have on the immune cell response to cancer. Specifically, anti-CD137 antibodies stimulate and activate effector T lymphocytes (e.g., by stimulating CD8+T lymphocytes to produce INFT), and enhance production of NKTs, and APCs (e.g., macrophages).

Urelumab demonstrated promising results in preclinical experiments and early clinical studies (Sznol et al., Clin. Oncol. 2008; 26 (Suppl. 15). However, in later studies, Urelumab demonstrated liver toxicity resulting in pausing development of the antibody until February 2012 (Segal et al., Clin. Cancer Res. 2017; 23:1929-1936). The liver toxicity was mostly due to S100A4 protein secreted by tumor and stromal cells, and studies that dose limited Urelumab to 8 mg or 0.1 mg/kg per patient for every 3 weeks have restored interest in this antibody (Segal et al., Clin. Cancer Res. 2017; 23:1929-1936).

In contrast with Urelumab, Utomilumab showed a better safety profile and initial studies show no liver toxicity or other dose limiting factors (Segal et al., J. Clin. Oncol. 2014; 32 (Suppl. 15). The reported outcomes from a phase I trial of Utomulumab as monotherapy indicated a good safety profile (Segal et al., Clin. Cancer Res. 2018; 24:1816-1823). The difference between the two antibodies has been speculated to be due to their different binding sites on the CD137 receptor.

There are no approved therapeutic antibodies against CD137, and there remains an unmet medical need for therapeutics targeting CD137. In addition, anti-TAA×CD137 multispecific antibodies that recruit immune cells to tumor associated antigen (TAA) expressing cancers would be useful in the treatment of cancer.

### SUMMARY OF THE DISCLOSURE

The present disclosure contains antibodies and antigen-binding fragments specifically binds human CD137. In addition, the CD137 VHH domain fragments disclosed herein can be used to construct multispecific antibodies with other modalities such as TAAs, immune checkpoints or immune stimulators. CD137 antibodies alone or in combination with other modalities could potentially be used for the treatment or prevention of cancer, autoimmune disease or infectious diseases.

Also, the present disclosure is directed to multispecific anti-GPC3×CD137 antibodies and antigen-binding fragments thereof.

The present disclosure encompasses the following embodiments.

Embodiment 1: An antibody or antigen-binding fragment thereof which specifically binds human CD137, wherein:
(1) the antibody or antigen-binding fragment thereof specifically binds to an epitope comprising or consisting of amino acid residues Phe36, Asp38, Pro49, Pro50, Asn51, Thr61, Cys62, Asp63, Ile64, Gln67 of human CD137 (SEQ ID NO:35);
(2) the antibody or antigen-binding fragment thereof specifically binds to an epitope comprising or consisting of amino acid residues Ser55, Ala56, Arg75, Glu85, Ala97 and Gly98 of human CD137 (SEQ ID NO:35); or
(3) the antibody or antigen-binding fragment thereof specifically binds to a human CD137 dimer comprising or consisting of a first human CD137 monomer and a second human CD137 monomer, wherein the antibody or antigen-binding fragment thereof specifically binds to an epitope of the first human CD137 monomer (SEQ ID NO:35) comprising or consisting of amino acid residues Phe36, Asp38, Pro49, Pro50, Asn51, Thr61, Cys62, Asp63, Ile64, Gln67, and the antibody or antigen-binding fragment thereof specifically binds to an epitope of the second human CD137 monomer (SEQ ID NO:35) comprising or consisting of amino acid residues Ser55, Ala56, Arg75, Glu85, Ala97 and Gly98; and/or the antibody or antigen-binding fragment thereof binds to a human CD137 dimer and promotes human CD137 clustering.

Embodiment 2. An antibody or antigen-binding fragment thereof which specifically binds human CD137, comprising:
(i) a heavy chain variable region (VH) that comprises (a) a HCDR1 (Heavy Chain Complementarity Determining Region 1) of SEQ ID NO: 1, (b) a HCDR2 of SEQ ID NO: 2, and (c) a HCDR3 of SEQ ID NO: 3; or
(ii) a heavy chain variable region that comprises (a) a HCDR1 of SEQ ID NO:1, (b) a HCDR2 of SEQ ID NO: 10, and (c) a HCDR3 of SEQ ID NO: 3.

Embodiment 3. The antibody or antigen-binding fragment thereof of any one of embodiments 1-2, that comprises:
(i) a heavy chain variable region (VH) comprising an amino acid sequence at least 90, 91, 92, 93, 94, 95, 96, 97, 98 or 99% identical to SEQ ID NO: 17;
(ii) a heavy chain variable region (VH) comprising an amino acid sequence at least 90, 91, 92, 93, 94, 95, 96, 97, 98 or 99% identical to SEQ ID NO: 11;
(iii) a heavy chain variable region (VH) comprising an amino acid sequence at least 90, 91, 92, 93, 94, 95, 96, 97, 98 or 99% identical to SEQ ID NO: 13;
(iv) a heavy chain variable region (VH) comprising an amino acid sequence at least 90, 91, 92, 93, 94, 95, 96, 97, 98 or 99% identical to SEQ ID NO: 15; or
(v) a heavy chain variable region (VH) comprising an amino acid sequence at least 90, 91, 92, 93, 94, 95, 96, 97, 98 or 99% identical to SEQ ID NO: 4.

Embodiment 4. The antibody or antigen-binding fragment thereof of embodiment 3, wherein one, two, three, four, five, six, seven, eight, nine, or ten amino acids within SEQ ID NO: 17, SEQ ID NO: 11, SEQ ID NO: 13, SEQ ID NO: 15, or SEQ ID NO: 4 have been inserted, deleted or substituted.

Embodiment 5. The antibody or antigen-binding fragment thereof of any one of the preceding embodiments, that comprises:
(i) a heavy chain variable region (VH) that comprises SEQ ID NO: 17;
(ii) a heavy chain variable region (VH) that comprises SEQ ID NO: 11;
(iii) a heavy chain variable region (VH) that comprises SEQ ID NO: 13
(iv) a heavy chain variable region (VH) that comprises SEQ ID NO: 15; or
(v) a heavy chain variable region (VH) that comprises SEQ ID NO: 4.

Embodiment 6. The antibody or antigen-binding fragment thereof of any one of the preceding embodiments, which is a monoclonal antibody, a chimeric antibody, a humanized antibody, a human engineered antibody, a single chain antibody (scFv), a Fab fragment, a Fab′ fragment, or a F(ab′)2 fragment.

Embodiment 7. The antibody or antigen-binding fragment thereof of any one of the preceding embodiments, wherein the antibody or antigen-binding fragment thereof comprises a heavy chain constant region of the subclass of IgG1, IgG2, IgG3, or IgG4, and/or a light chain constant region of the type of kappa or lambda.

Embodiment 8. The antibody or antigen-binding fragment thereof of any one of the preceding embodiments, wherein the antibody or antigen-binding fragment thereof has antibody dependent cellular cytotoxicity (ADCC) or complement dependent cytotoxicity (CDC).

Embodiment 9. The antibody or antigen-binding fragment thereof of any one of the preceding embodiments, wherein the antibody or antigen-binding fragment thereof has reduced glycosylation or no glycosylation or is hypofucosylated.

Embodiment 10. The antibody or antigen-binding fragment thereof of any one of the preceding embodiments, wherein the antibody or antigen-binding fragment thereof comprises increased bisecting GlcNac structures.

Embodiment 11. The antibody or antigen-binding fragment thereof of any one of the preceding embodiments, wherein the antibody or antigen-binding fragment thereof comprises a Fc domain, and wherein the Fc domain is an IgG1 Fc with reduced effector function, optionally the Fc domain comprises an amino acid sequence of SEQ ID NO: 19 or SEQ ID NO: 53.

Embodiment 12. The antibody or antigen-binding fragment thereof of any one of the preceding embodiments, wherein the antibody or antigen-binding fragment thereof comprises a Fc domain, and wherein the Fc domain is an IgG1 Fc with reduced effector function and/or extended half-life, optionally the Fc domain comprises an amino acid sequence of SEQ ID NO: 20.

Embodiment 13. A pharmaceutical composition comprising the antibody or antigen-binding fragment thereof of any one of the preceding embodiments and a pharmaceutically acceptable carrier.

Embodiment 14. A method of treating a cancer comprising administering to a patient in need thereof a therapeutically effective amount of the antibody or antigen-binding fragment thereof of any one of embodiments 1-12, or the pharmaceutical composition of embodiment 13.

Embodiment 15. The method of embodiment 14, wherein the cancer is gastric cancer, colon cancer, pancreatic cancer, breast cancer, head and neck cancer, kidney cancer, liver cancer, lung cancer, small cell lung cancer, non-small cell lung cancer, ovarian cancer, skin cancer, mesothelioma, lymphoma, leukemia, myeloma and sarcoma.

Embodiment 16. The method of any one of embodiments 14-15, wherein the antibody or antigen-binding fragment thereof is administered in combination with another therapeutic agent.

Embodiment 17. The method of embodiment 16, wherein the therapeutic agent is an anti-PD-1 antibody.

Embodiment 18. The method of embodiment 17, wherein the anti-PD1 antibody is Tislelizumab.

Embodiment 19: A multispecific antibody or antigen-binding fragment thereof comprising at least a first antigen binding domain that specifically binds a human tumor-associated antigen (TAA), and at least a second antigen binding domain that specifically binds human CD137, wherein the second antigen binding domain is:
(1) an antibody or antigen-binding fragment thereof specifically binds to an epitope comprising or consisting of amino acid residues Phe36, Asp38, Pro49, Pro50, Asn51, Thr61, Cys62, Asp63, Ile64, Gln67 of human CD137 (SEQ ID NO:35);
(2) an antibody or antigen-binding fragment thereof specifically binds to an epitope comprising or consisting of amino acid residues Ser55, Ala56, Arg75, Glu85, Ala97 and Gly98 of human CD137 (SEQ ID NO:35); or
(3) an antibody or antigen-binding fragment thereof specifically binds to a human CD137 dimer comprising or consisting of a first human CD137 monomer and a second human CD137 monomer, wherein the antibody or antigen-binding fragment thereof specifically binds to an epitope of the first human CD137 monomer (SEQ ID NO:35) comprising or consisting of amino acid residues Phe36, Asp38, Pro49, Pro50, Asn51, Thr61, Cys62, Asp63, Ile64, Gln67, and the antibody or antigen-binding fragment thereof specifically binds to an epitope of the second human CD137 monomer (SEQ ID NO:35) comprising or consisting of amino acid residues Ser55, Ala56, Arg75, Glu85, Ala97 and Gly98; and/or the antibody or antigen-binding fragment thereof binds to a human CD137 dimer and promotes human CD137 clustering.

Embodiment 20. A multispecific antibody or antigen-binding fragment thereof, comprising at least a first antigen binding domain that specifically binds a human tumor-associated antigen (TAA), and at least a second antigen binding domain that specifically binds human CD137, wherein the second antigen binding domain comprises:
(i) a heavy chain variable region (VH) that comprises (a) a HCDR1 of SEQ ID NO: 1, (b) a HCDR2 of SEQ ID NO: 2, and (c) a HCDR3 of SEQ ID NO: 3; or
(ii) a heavy chain variable region (VH) that comprises (a) a HCDR1 of SEQ ID NO: 1, (b) a HCDR2 of SEQ ID NO: 10, and (c) a HCDR3 of SEQ ID NO: 3.

Embodiment 21. The multispecific antibody or antigen-binding fragment thereof of any one of embodiments 19-20, wherein the second antigen binding domain specifically binds human CD137 comprises:
(i) a heavy chain variable region (VH) comprising an amino acid sequence at least 90, 91, 92, 93, 94, 95, 96, 97, 98 or 99% identical to SEQ ID NO: 17;
(ii) a heavy chain variable region (VH) comprising an amino acid sequence at least 90, 91, 92, 93, 94, 95, 96, 97, 98 or 99% identical to SEQ ID NO: 11;
(iii) a heavy chain variable region (VH) comprising an amino acid sequence at least 90, 91, 92, 93, 94, 95, 96, 97, 98 or 99% identical to SEQ ID NO: 13;
(iv) a heavy chain variable region (VH) comprising an amino acid sequence at least 90, 91, 92, 93, 94, 95, 96, 97, 98 or 99% identical to SEQ ID NO: 15; or
(v) a heavy chain variable region (VH) comprising an amino acid sequence at least 90, 91, 92, 93, 94, 95, 96, 97, 98 or 99% identical to SEQ ID NO: 4.

Embodiment 22. The multispecific antibody or antigen-binding fragment thereof of any one of embodiments 19-21, wherein the second antigen binding domain specifically binds human CD137 comprises:
(i) a heavy chain variable region (VH) that comprises SEQ ID NO: 17;
(ii) a heavy chain variable region (VH) that comprises SEQ ID NO: 11;
(iii) a heavy chain variable region (VH) that comprises SEQ ID NO: 13
(iv) a heavy chain variable region (VH) that comprises SEQ ID NO: 15; or
(v) a heavy chain variable region (VH) that comprises SEQ ID NO: 4.

Embodiment 23. The multispecific antibody or antigen-binding fragment thereof of any one of embodiments 19-22, wherein the TAA is GPC3.

Embodiment 24. A multispecific antibody or antigen-binding fragment thereof, comprising a first antigen binding domain that specifically binds to human Glypican 3 (GPC3) and a second antigen binding domain that specifically binds to human CD137.

Embodiment 25: The multispecific antibody or antigen-binding fragment thereof of embodiment 24, wherein the second antigen binding domain that specifically binds to human CD137 is:
(1) an antibody or antigen-binding fragment thereof specifically binds to an epitope comprising or consisting of amino acid residues Phe36, Asp38, Pro49, Pro50, Asn51, Thr61, Cys62, Asp63, Ile64, Gln67 of human CD137 (SEQ ID NO:35);
(2) an antibody or antigen-binding fragment thereof specifically binds to an epitope comprising or consisting of amino acid residues Ser55, Ala56, Arg75, Glu85, Ala97 and Gly98 of human CD137 (SEQ ID NO:35); or
(3) an antibody or antigen-binding fragment thereof specifically binds to a human CD137 dimer comprising or consisting of a first human CD137 monomer and a second human CD137 monomer, wherein the antibody or antigen-binding fragment thereof specifically binds to an epitope of the first human CD137 monomer (SEQ ID NO:35) comprising or consisting of amino acid residues Phe36, Asp38, Pro49, Pro50, Asn51, Thr61, Cys62, Asp63, Ile64, Gln67, and the antibody or antigen-binding fragment thereof specifically binds to an epitope of the second human CD137 monomer (SEQ ID NO:35) comprising or consisting of amino acid residues Ser55, Ala56, Arg75, Glu85, Ala97 and Gly98; and/or the antibody or antigen-binding fragment thereof binds to a human CD137 dimer and promotes human CD137 clustering.

Embodiment 26. The multispecific antibody or antigen-binding fragment thereof of any one of embodiments 24-25, wherein the second antigen binding domain that specifically binds to human CD137 comprises:
(i) a heavy chain variable region (VH) that comprises (a) a HCDR1 of SEQ ID NO: 1, (b) a HCDR2 of SEQ ID NO: 2, and (c) a HCDR3 of SEQ ID NO: 3; or
(ii) a heavy chain variable region (VH) that comprises (a) a HCDR1 of SEQ ID NO: 1, (b) a HCDR2 of SEQ ID NO: 10, and (c) a HCDR3 of SEQ ID NO: 3.

Embodiment 27. The multispecific antibody or antigen-binding fragment thereof of embodiment 26, wherein the second antigen binding domain that specifically binds to human CD137 comprises:
(i) a heavy chain variable region (VH) comprising an amino acid sequence at least 90, 91, 92, 93, 94, 95, 96, 97, 98 or 99% identical to SEQ ID NO: 17;
(ii) a heavy chain variable region (VH) comprising an amino acid sequence at least 90, 91, 92, 93, 94, 95, 96, 97, 98 or 99% identical to SEQ ID NO: 11;
(iii) a heavy chain variable region (VH) comprising an amino acid sequence at least 90, 91, 92, 93, 94, 95, 96, 97, 98 or 99% identical to SEQ ID NO: 13;
(iv) a heavy chain variable region (VH) comprising an amino acid sequence at least 90, 91, 92, 93, 94, 95, 96, 97, 98 or 99% identical to SEQ ID NO: 15; or
(v) a heavy chain variable region (VH) comprising an amino acid sequence at least 90, 91, 92, 93, 94, 95, 96, 97, 98 or 99% identical to SEQ ID NO: 4.

Embodiment 28. The multispecific antibody or antigen-binding fragment thereof of embodiment 27, wherein one, two, three, four, five, six, seven, eight, nine, or ten amino acids within SEQ ID NO: 17, SEQ ID NO: 11, SEQ ID NO: 13, SEQ ID NO: 15, or SEQ ID NO: 4 have been inserted, deleted or substituted.

Embodiment 29. The multispecific antibody or antigen-binding fragment thereof of any one of embodiments 24-28, wherein the second antigen binding domain that specifically binds to human CD137 comprises:
(i) a heavy chain variable region (VH) that comprises SEQ ID NO: 17;
(ii) a heavy chain variable region (VH) that comprises SEQ ID NO: 11;
(iii) a heavy chain variable region (VH) that comprises SEQ ID NO: 13
(iv) a heavy chain variable region (VH) that comprises SEQ ID NO: 15; or
(v) a heavy chain variable region (VH) that comprises SEQ ID NO: 4.

Embodiment 30. The multispecific antibody or antigen-binding fragment thereof of any one of embodiments 19-29, wherein the first antigen binding domain that specifically binds to human GPC3 comprises:
a heavy chain variable region (VH) that comprises (a) a HCDR1 of SEQ ID NO: 45, (b) a HCDR2 of SEQ ID NO: 46, and (c) a HCDR3 of SEQ ID NO: 47; and
a light chain variable region (VL) that comprises (d) a LCDR1 of SEQ ID NO: 48, (e) a LCDR2 of SEQ ID NO: 49, and (f) a LCDR3 of SEQ ID NO: 50.

Embodiment 31. The multispecific antibody or antigen-binding fragment thereof of any one of embodiments 19-30, wherein the first antigen binding domain that specifically binds to human GPC3 comprises:
a heavy chain variable region (VH) comprising an amino acid sequence at least 90, 91, 92, 93, 94, 95, 96, 97, 98 or 99% identical to SEQ ID NO: 41, and a light chain variable region (VL) comprising an amino acid sequence at least 90, 91, 92, 93, 94, 95, 96, 97, 98 or 99% identical to SEQ ID NO: 43.

Embodiment 32. The multispecific antibody or antigen-binding fragment thereof of embodiment 31, wherein one, two, three, four, five, six, seven, eight, nine, or ten amino acids within SEQ ID NO: 41 or SEQ ID NO: 43 have been inserted, deleted or substituted.

Embodiment 33. The multispecific antibody or antigen-binding fragment thereof of any one of embodiments 19-32, wherein the first antigen binding domain that specifically binds to human GPC3 comprises: a heavy chain variable region (VH) that comprises SEQ ID NO: 41, and a light chain variable region (VL) that comprises SEQ ID NO: 43.

Embodiment 34. The multispecific antibody or antigen-binding fragment thereof of any one of embodiments 19-33, wherein the first antigen binding domain that specifically binds to human GPC3 comprises
a heavy chain variable region (VH) that comprises (a) a HCDR1 of SEQ ID NO: 45, (b) a HCDR2 of SEQ ID NO: 46, and (c) a HCDR3 of SEQ ID NO: 47; and
a light chain variable region (VL) that comprises (d) a LCDR1 of SEQ ID NO: 48, (e) a LCDR2 of SEQ ID NO: 49, and (f) a LCDR3 of SEQ ID NO: 50, and
wherein the second antigen binding domain that specifically binds to human CD137 comprises:
(i) a heavy chain variable region (VH) that comprises (a) a HCDR1 of SEQ ID NO: 1, (b) a HCDR2 of SEQ ID NO: 2, and (c) a HCDR3 of SEQ ID NO: 3; or
(ii) a heavy chain variable region (VH) that comprises (a) a HCDR1 of SEQ ID NO: 1, (b) a HCDR2 of SEQ ID NO: 10, and (c) a HCDR3 of SEQ ID NO: 3.

Embodiment 35. The multispecific antibody or antigen-binding fragment thereof of any one of embodiments 19-34, wherein the first antigen binding domain that specifically binds to human GPC3 comprises:
a heavy chain variable region (VH) that comprises SEQ ID NO: 41, and a light chain variable region (VL) that comprises SEQ ID NO: 43; and
wherein the second antigen binding domain that specifically binds to human CD137 comprises:
(i) a heavy chain variable region (VH) that comprises SEQ ID NO: 4;
(ii) a heavy chain variable region (VH) that comprises SEQ ID NO: 11;
(iii) a heavy chain variable region (VH) that comprises SEQ ID NO: 13;
(iv) a heavy chain variable region (VH) that comprises SEQ ID NO: 15; or
(v) a heavy chain variable region (VH) that comprises SEQ ID NO: 17.

Embodiment 36. The multispecific antibody or antigen-binding fragment thereof of any one of embodiments 19-35, wherein the multispecific antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)2 fragment.

Embodiment 37. The multispecific antibody or antigen-binding fragment thereof of any one of the embodiments 19-36, wherein the first antigen binding domain that specifically binds to human GPC3 is a monoclonal antibody, a chimeric antibody, a humanized antibody, a human engineered antibody, a single chain antibody (scFv), a single domain antibody, a Fab fragment, a Fab′ fragment, or a F(ab′)2 fragment, and the second antigen binding domain that specifically binds to human CD137 is a monoclonal antibody, a chimeric antibody, a humanized antibody, a human engineered antibody, a single chain antibody (scFv), a single domain antibody, a Fab fragment, a Fab′ fragment, or a F(ab′)2 fragment.

Embodiment 38. The multispecific antibody or antigen-binding fragment thereof of any one of embodiments 19-37, wherein the multispecific antibody or antigen-binding fragment thereof is a bispecific antibody.

Embodiment 39. The multispecific antibody or antigen-binding fragment thereof of embodiment 38, wherein the bispecific antibody is in 2+2 format.

Embodiment 40. The multispecific antibody or antigen-binding fragment thereof of any one of embodiments 19-39, wherein the multispecific antibody or antigen-binding fragment thereof contains a linker from SEQ ID NO: 60 to SEQ ID NO: 101.

Embodiment 41. The multispecific antibody or antigen-binding fragment thereof of embodiment 40, wherein the linker is SEQ ID NO: 62.

Embodiment 42. The multispecific antibody or antigen-binding fragment thereof of embodiment 40, wherein the linker is SEQ ID NO: 67.

Embodiment 43. The multispecific antibody or antigen-binding fragment thereof of any one of embodiments 19-42, wherein the multispecific antibody or antigen-binding fragment comprises a heavy chain constant region of the subclass of IgG1, IgG2, IgG3, or IgG4, and/or a light chain constant region of the type of kappa or lambda, and
wherein the heavy chain constant region comprises CH1 and/or Fc domain.

Embodiment 44. The multispecific antibody or antigen-binding fragment thereof of any one of embodiments 19-43, wherein the multispecific antibody or antigen-binding fragment thereof has antibody dependent cellular cytotoxicity (ADCC) or complement dependent cytotoxicity (CDC).

Embodiment 45. The multispecific antibody or antigen-binding fragment thereof of any one of embodiments 19-44, wherein the multispecific antibody or antigen-binding fragment thereof has reduced glycosylation or no glycosylation or is hypofucosylated.

Embodiment 46. The multispecific antibody or antigen-binding fragment thereof of any one of embodiments 19-45, wherein the multispecific antibody or antigen-binding fragment thereof comprises increased bisecting GlcNac structures.

Embodiment 47. The multispecific antibody or antigen-binding fragment thereof of any one of embodiments 19-46, wherein the multispecific antibody or antigen-binding fragment thereof comprises a Fc domain, and wherein the Fc domain is an IgG1 Fc with reduced effector function, optionally the Fc domain comprises an amino acid sequence of SEQ ID NO: 53.

Embodiment 48. The multispecific antibody or antigen-binding fragment thereof of any one of embodiments 19-47, wherein the multispecific antibody or antigen-binding fragment thereof comprises a Fc domain, and wherein the Fc domain is an IgG1 Fc with reduced effector function and/or extended half-life, optionally the Fc domain comprises an amino acid sequence of SEQ ID NO: 20.

Embodiment 49. The multispecific antibody or antigen-binding fragment thereof of any one of embodiments 19-48, wherein the multispecific antibody or antigen-binding fragment thereof comprises a Fc domain, and wherein the Fc domain is an IgG4 Fc.

Embodiment 50. The multispecific antibody or antigen-binding fragment thereof of any one of embodiments 19-49, wherein:
a) the heavy chain variable region (VH) of the first antigen binding domain that specifically binds to human GPC3, a CH1 domain, the Fc domain, and the heavy chain variable region (VH) of the second antigen binding domain that specifically binds to human CD137 are arranged in a first polypeptide in the direction of N terminal to C terminal;
optionally, C terminal of the Fc domain is linked to N terminal of the heavy chain variable region (VH) of the second antigen binding domain via a linker; and
b) the light chain variable region (VL) of the first antigen binding domain that specifically binds to human GPC3 and a first light chain constant region are arranged in a second polypeptide in the direction of N terminal to C terminal.

Embodiment 51. The multispecific antibody or antigen-binding fragment thereof of any one of embodiments 19-50, wherein the multispecific antibody or antigen-binding fragment comprises
(i) a first polypeptide of SEQ ID NO: 25 and a second polypeptide of SEQ ID NO: 23;
(ii) a first polypeptide of SEQ ID NO: 21 and a second polypeptide of SEQ ID NO: 23;
(iii) a first polypeptide of SEQ ID NO: 33 and a second polypeptide of SEQ ID NO: 23;
(iv) a first polypeptide of SEQ ID NO: 27 and a second polypeptide of SEQ ID NO: 23;
(v) a first polypeptide of SEQ ID NO: 29 and a second polypeptide of SEQ ID NO: 23; or
(vi) a first polypeptide of SEQ ID NO: 31 and a second polypeptide of SEQ ID NO: 23.

Embodiment 52. A pharmaceutical composition comprising the multispecific antibody or antigen-binding fragment thereof of any one of embodiments 19-51 and a pharmaceutically acceptable carrier.

Embodiment 53. A method of treating a cancer comprising administering to a patient in need thereof a therapeutically effective amount of the multispecific antibody or antigen-binding fragment thereof of any one of embodiments 19-51, or the pharmaceutical composition of embodiment 52.

Embodiment 54. The method of embodiment 53, wherein the cancer is an advanced or metastatic solid tumor.

Embodiment 55. The method of any one of embodiments 53-54, wherein the cancer expresses GPC3.

Embodiment 56. The method of any one of embodiments 53-55, wherein the cancer is liver cancer, lung cancer, gastric cancer, germ cell tumors, thyroid cancer, pancreatic cancer, ovarian cancer, skin cancer, kidney cancer, esophageal carcinoma, atypical teratoid rhabdoid tumor of the brain, or undifferentiated synovial sarcoma.

Embodiment 57. The method of embodiment 56, wherein the liver cancer is hepatoblastoma or hepatocellular carcinoma (HCC).

Embodiment 58. The method of embodiment 56, wherein the lung cancer is non-small cell lung cancer (NSCLC) or small cell lung cancer (SCLC).

Embodiment 59. The method of embodiment 58, wherein the non-small cell lung cancer is squamous non-small cell lung cancer.

Embodiment 60. The method of embodiment 58, wherein the non-small cell lung cancer is GPC3+ squamous non-small cell lung cancer.

Embodiment 61. The method of embodiment 56, wherein the gastric cancer is alpha fetoprotein+ (AFP+) gastric cancer.

Embodiment 62. The method of embodiment 56, wherein the kidney cancer is Wilms tumor.

Embodiment 63. The method of embodiment 56, wherein the esophageal carcinoma is esophageal squamous cell carcinoma.

Embodiment 64. The method of embodiment 56, wherein the esophageal carcinoma is GPC3+ esophageal squamous cell carcinoma.

Embodiment 65. The method of embodiment 56, wherein the germ cell tumor is yolk sac tumors or non-dysgerminomas.

Embodiment 66. The method of any one of embodiments 53-65, wherein the multispecific antibody or antigen-binding fragment thereof, or the pharmaceutical composition is administered in combination with another therapeutic agent.

Embodiment 67. The method of embodiment 66, wherein the therapeutic agent an anti-PD1 or anti-PDL1 antibody.

Embodiment 68. The method of embodiment 67, wherein the anti-PD1 antibody is Tislelizumab.

Embodiment 69. An isolated nucleic acid that encodes the antibody, multispecific antibody or antigen-binding fragment thereof of any one of embodiments 1-12 and 19-51.

Embodiment 70. A vector comprising the nucleic acid of embodiment 69.

Embodiment 71. A host cell comprising the nucleic acid of embodiment 69 or the vector of embodiment 70.

Embodiment 72. A process for producing a multispecific antibody or antigen-binding fragment thereof comprising cultivating the host cell of embodiment 71 and recovering the antibody or antigen-binding fragment thereof from the culture.

In some embodiments, the present disclosure provides anti-CD137 antibodies or antigen-binding fragments thereof which show specific binding and high affinity to human CD137 and cynomolgus monkey CD137, show superior overall biophysical properties, e.g., Tm or Tagg, show superior pharmacokinetics, and/or is capable of binding to CD137 dimer and promoting CD137 clustering.

In some embodiments, the present disclosure provides anti-CD137 antibodies or antigen-binding fragments thereof having at least one or more of the following features:
(1) showing specific binding and high affinity to human CD137 and cynomolgus monkey CD137;
(2) having superior pharmacokinetics;
(3) having superior overall biophysical properties, e.g., Tm or Tagg, and/or having superior overall stability;
(4) being a humanized antibodies having low immunogenicity risk to human; and
(5) is capable of binding to CD137 dimer and promoting CD137 clustering.

In some embodiments, the present disclosure provides anti-CD137 antibodies or antigen-binding fragments thereof which are humanized antibodies having low immunogenicity risk to human, while maintain specific binding and high affinity to human CD137 and cynomolgus monkey CD137 and show superior overall biophysical properties and/or stability. In some embodiments, the present disclosure provides anti-CD137 antibodies or antigen-binding fragments thereof showing specific binding and high affinity to human CD137 and cynomolgus monkey CD137 and/or capable of binding to CD137 dimer and promoting CD137 clustering (e.g., via CDR residues).

In some embodiment, the present disclosure provides a GPC3×CD137 multispecific antibody or antigen-binding fragment thereof having at least one or more of the following features:
(1) showing specific binding and high affinity to human CD137 and cynomolgus monkey CD137;
(2) having specific binding and high affinity to human GPC3 and cynomolgus monkey GPC3, and showing high affinity to wide range of GPC expression (low to high expression);
(3) inducing T cell activation including cytokine release (e.g., IFN-7 or IL-2) and T cell killing activity in a GPC3 dependent manner, and reduced T cell activation or T cell killing activity in the absence of GPC3 expressing cells;
(4) inducing T cell activation and potent T cell killing activity on wide range of GPC3 expressing (low, medium, high expression) cells;
(5) inhibits tumor growth effectively when it is administered alone;
(7) induces synergistic effects (e.g., tumor growth inhibition and/or tumor free ratio) when it is administered with anti-PD-1 antibody;
(8) having excellent pharmacokinetics;
(9) having excellent overall biophysical properties, e.g., Tm or Tagg, and/or stability; and
(10) is capable of binding to CD137 dimer and promoting CD137 clustering.

### BRIEF DESCRIPTION OF THE DRAWINGS

FIG. 1 demonstrates BGA-9612 bound with huCD137 and partially competed with 20 μg/ml CD137L in comparison with Urelumab (BMS-663513) by ELISA.

FIG. 2 shows a comparison of the FACS binding affinities between BGA-9612 and other humanized VHHs in human CD137 overexpressing HuT78 cells.

FIG. 3 is a schematic diagram of the design of a tumor-targeting GPC3×CD137 multispecific antibody format.

FIG. 4A shows the binding assay of BE-774 to CD137 overexpressing cells Hut78/CD137 by flow cytometry, demonstrating the binding of BE-774 to native huCD137 expressed on the cell surface. FIG. 4B shows the binding assay of BE-774 to GPC3 expressing cells HepG2 by flow cytometry, demonstrating the binding of BE-774 to native huGPC3 expressed on cell surface.

FIGS. 5A-5B demonstrate BE-774 and BE-653 induced T cell activation when co-cultured with GPC3 positive tumor cells HepG2. FIG. 5A is a schematic diagram of CD137 (41BB) activation via co-stimulating huPBMCs with BE-774 or BE-653 and OS8-expressing hepatocellular carcinoma (HCC) cell lines. FIG. 5B shows BE-774 and BE-653 induced dose-dependent cytokine release in PBMC co-cultured with HepG2 cells but not with GPC3 negative cells.

FIGS. 6A-6B demonstrate BE-774 and BE-653 enhanced T cell killing activity to GPC3 positive tumor cells HepG2. FIG. 6A is a schematic diagram of CD137 (4-1BB) activation via co-stimulating huPBMCs with BE-774 or BE-653 in combination with an EpCAM/CD3 bispecific T-cell engager (BiTE) which provides a first signal for T cell activation. FIG. 6B shows BE-774 and BE-653 dose-dependently enhanced T cell killing activity to GPC3 expressing cells but not to GPC3 negative cells.

FIG. 7 shows the PK profile of BE-933 and BE-774 in cyno.

FIG. 8 shows the PK profile of BE-933 and BE-774 in a hFcRn mouse model.

FIG. 9A-9B shows binding of BE-915 to human CD137 overexpressing on Hut78 (FIG. 9B) and human GPC3 expressing on HepG2 (FIG. 9A).

FIG. 10 shows the binding specificity of BE-915 with CD137 and other TNFRSF members.

FIG. 11A-11B shows that BE-915 cross-competes with CD137L on binding to human CD137. CD137L blocks the binding of BE-915 and CD137 expressing on HuT78 (FIG. 11A). BE-915 blocks the binding of CD137L and CD137 expressing on HuT78 (FIG. 11B).

FIG. 12A-12C shows that BE-915 induces the IL-2 and IFN-7 release from human PBMCs. FIG. 12A is a schematic diagram of CD137 activation via co-stimulating huPBMCs with BE-915 and OS8-expressing hepatocellular carcinoma (HCC) cell lines. FIG. 12B-12C shows BE-915 induced dose-dependent cytokine release in PBMC in a GPC3 expression dependent manner. PBMCs from two donors were tested.

FIG. 13A-13C shows BE-915 induces T-cell killing activity of human PBMCs.

FIG. 13A is a schematic diagram of CD137 activation via co-stimulating huPBMCs with BE-915 in combination with an EpCAM/CD3 bispecific T-cell engager (BiTE) which provides a first signal for T cell activation. FIG. 13B-C shows BE-915 dose-dependently enhanced T cell killing activity to GPC3 expressing cells but not to GPC3 negative cells. PBMCs from two donors were tested.

FIG. 14 shows the pharmacokinetics profile of BE-915 in Cynomolgus after i.v infusion (5 mg/kg, N=2).

FIG. 15 shows the efficacy of BE-915 monotherapy in MC38/hGPC3 model in humanized CD137 knock-in mice.

FIG. 16 shows the efficacy of the combination of BE-915 and anti-PD-1 antibody in LL/2/hGPC3 model in humanized CD137 knock-in mice.

FIG. 17 shows a schematic diagram of partially competitive binding of VHH (BGA-2524) against CD137L for CD137. The crystal structure of VHH (BGA-2524)/CD137 was superposed with CD137L/CD137 complex (PDB: 6MGP) via CD137 CRD1 and CRD2 domain. The CD137, CD137L and VHH (BGA-2524) are colored in black, white and gray, respectively.

FIG. 18 shows VHH (BGA-2524) binds CD137 dimer. The crystal structure analysis shows that VHH (BGA-2524) has the capability of binding CD137 dimer to promote CD137 clustering. Each monomer of CD137 dimer is shown in white or gray, with VHH (BGA-2524) in black cartoon on the surface (left). The epitope of VHH (BGA-2524) is shown in black on the surface of the CD137 dimer (right, with BGA-2524 removed).

FIG. 19 shows the atomic interactions on the binding surface of VHH (BGA-2524)/CD137 complex. The binding interface between VHH (BGA-2524) and CD137 identifies certain key residues of VHH (BGA-2524) (paratope residues, amino acid underlined) and CD137 (epitope residues). Each monomer of CD137 dimer is shown in white or gray cartoon covered with transparent surface, alongside CRD1, CRD2 and CRD3 domain indicated with line, respectively. The paratope residues are shown in black line with amino acid underlined (most of framework region removed).

### DETAILED DESCRIPTION

The present disclosure provides for anti-CD137 antibodies and antigen-binding fragments thereof, and multispecific antibody or antigen-binding fragment thereof that recognize CD137 as one antigen and at least one tumor associated antigen (TAA) as the other antigen. The present disclosure also provides for anti-GPC3×CD137 multispecific antibodies and antigen-binding fragments thereof. Furthermore, the present disclosure provides antibodies that have desirable pharmacokinetic characteristics, desirable biophysical properties, and other desirable attributes, and thus can be used for reducing the likelihood of or treating cancer. The present disclosure further provides pharmaceutical compositions comprising the antibodies and methods of making and using such pharmaceutical compositions for the prevention and treatment of cancer and associated disorders.

### I. Anti-GPC3 Antibodies

The present disclosure provides for antibodies or antigen-binding fragments thereof that specifically bind to human GPC3. In one embodiment, the anti-GPC3 antibodies or antigen-binding fragments thereof specifically bind to human GPC3 with a binding affinity (K_{D}) of from 1×10⁻⁶ M to 1×10⁻¹⁰ M. In another embodiment, the anti-GPC3 antibodies or antigen-binding fragments thereof bind to human GPC3 with a binding affinity (K_{D}) of about 1×10⁻⁶ M, about 1×10⁻⁷ M, about 1×10⁻⁸ M, about 1×10⁻⁹ M or about 1×10⁻¹0 M.

In one embodiment, the anti-GPC3 antibodies or antigen-binding fragments thereof comprise: a heavy chain variable region (VH) that comprises (a) a HCDR1 of SEQ ID NO: 45, (b) a HCDR2 of SEQ ID NO: 46, and (c) a HCDR3 of SEQ ID NO: 47; and a light chain variable region (VL) that comprises (d) a LCDR1 of SEQ ID NO: 48, (e) a LCDR2 of SEQ ID NO: 49, and (f) a LCDR3 of SEQ ID NO: 50, according to the Kabat numbering.

In another embodiment, the anti-GPC3 antibodies or antigen-binding fragments thereof comprise: a HCDR1, a HCDR2 and a HCDR3 from the heavy chain variable region (VH) set forth in SEQ ID NO: 41; and a LCDR1, a LCDR2 and a LCDR3 from the light chain variable region (VL) set forth in SEQ ID NO: 43.

In another embodiment, the anti-GPC3 antibodies or antigen-binding fragments thereof further comprise no more than one, two, three, four or five amino acid deletions, insertions or substitutions in the CDR, preferably the amino acid substitutions are conservative amino acid substitutions, while maintaining binding specificity and affinity.

In another embodiment, the anti-GPC3 antibodies or antigen-binding fragments thereof comprise a heavy chain variable region (VH) comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% identical to SEQ ID NO: 41, and a light chain variable region (VL) comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% identical to SEQ ID NO: 43. In another embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids within SEQ ID NO: 41 or SEQ ID NO: 43 have been inserted, deleted or substituted (optionally conservative amino acid substitutions). In another embodiment, such variations are in the framework region of the variable region. In another embodiment, anti-GPC3 antibodies or antigen-binding fragments thereof having such variations maintains binding specificity and affinity.

In another embodiment, the anti-GPC3 antibodies or antigen-binding fragments thereof comprise a heavy chain variable region (VH) that comprises SEQ ID NO: 41, and a light chain variable region (VL) that comprises SEQ ID NO: 43.

In another embodiment, the anti-human GPC3 antibodies or antigen-binding fragments thereof show a cross-species binding activity to cynomolgus GPC3.

### II. Anti-CD137 Antibodies

TABLE 1
Sequences of anti-CD137 antibodies

| Antibody | SEQ ID NO | | SEQUENCE |
|---|---|---|---|
| BGA-9612 | SEQ ID NO: 54 | HCDR1 | GRTFDNYA |
| (IMGT) | | (IMGT) | |
| | SEQ ID NO: 55 | HCDR2 | YWSGYA |
| | | (IMGT) | |
| | SEQ ID NO: 56 | HCDR3 | AISLKYPMATVTPDY |
| | | (IMGT) | |
| BGA-9612 | SEQ ID NO: 1 | HCDR1 | NYAVG |
| (Kabat) | | (Kabat) | |
| | SEQ ID NO: 2 | HCDR2 | TYWSGYAHYGDFENN |
| | | (Kabat) | |
| | SEQ ID NO: 3 | HCDR3 | SLKYPMATVTPDY |
| | | (Kabat) | |
| BGA-9612 | SEQ ID NO: 57 | HCDR1 | GRTFDNY |
| (Chothia) | | (Chothia) | |
| | SEQ ID NO: 58 | HCDR2 | WSGY |
| | | (Chothia) | |
| | SEQ ID NO: 59 | HCDR3 | SLKYPMATVTPDY |
| | | (Chothia) | |
| BGA-9612 | SEQ ID NO: 4 | VH AA | QVQLVESGGGTVQPGDSLRLSCAASGRTFDNYAVGWFRQ |
| | | | APGKERQYVGTYWSGYAHYGDFENNRFTISRDNAKNTVY |
| | | | LQMDNLKPEDTAVYYCAISLKYPMATVTPDYQGQGTQVT |
| | | | VSS |
| | SEQ ID NO: 5 | VH DNA | CAGGTGCAGCTCGTGGAGTCCGGGGGAGGAACGGTGC |
| | | | AGCCTGGGGACTCTCTGAGACTCTCCTGTGCTGCCTCTG |
| | | | GACGCACCTTCGATAACTATGCCGTGGGCTGGTTCCGCC |
| | | | AGGCTCCAGGGAAGGAGCGTCAGTATGTCGGCACGTAT |
| | | | TGGAGTGGTTACGCACACTATGGGGACTTTGAGAATAAC |
| | | | CGATTCACGATCTCCAGAGACAACGCCAAGAATACGGT |
| | | | GTATCTGCAAATGGACAACCTGAAACCTGAGGACACGG |
| | | | CCGTTTATTACTGTGCAATATCCCTTAAATATCCTATGGCG |
| | | | ACAGTTACCCCAGACTACCAGGGCCAGGGGACCCAGGT |
| | | | CACCGTCTCCTCA |
| BGA-6582 | SEQ ID NO: 54 | HCDR1 | GRTFDNYA |
| (IMGT) | | (IMGT) | |
| | SEQ ID NO: 55 | HCDR2 | YWSGYA |
| | | (IMGT) | |
| | SEQ ID NO: 56 | HCDR3 | AISLKYPMATVTPDY |
| | | (IMGT) | |
| BGA-6582 | SEQ ID NO: 1 | HCDR1 | NYAVG |
| (Kabat) | | (Kabat) | |
| | SEQ ID NO: 2 | HCDR2 | TYWSGYAHYGDFENN |
| | | (Kabat) | |
| | SEQ ID NO: 3 | HCDR3 | SLKYPMATVTPDY |
| | | (Kabat) | |
| BGA-6582 | SEQ ID NO: 57 | HCDR1 | GRTFDNY |
| (Chothia) | | (Chothia) | |
| | SEQ ID NO: 58 | HCDR2 | WSGY |
| | | (Chothia) | |
| | SEQ ID NO: 59 | HCDR3 | SLKYPMATVTPDY |
| | | (Chothia) | |
| BGA-6582 | SEQ ID NO: 8 | VH AA | EVQLLESGGGLVQPGGSLRLSCAASGRTFDNYAVGWFRQ |
| | | | APGKERQYVGTYWSGYAHYGDFENNRFTISRDNSKNTVY |
| | | | LQMNSLRAEDTAVYYCAISLKYPMATVTPDYQGQGTLVT |
| | | | VSS |
| | SEQ ID NO: 9 | VH DNA | GAAGTGCAGCTGCTGGAGTCCGGGGGCGGCCTGGTCCA |
| | | | ACCCGGGGGCAGCCTGAGACTGAGCTGCGCCGCCTCCG |
| | | | GCAGAACCTTCGACAACTACGCCGTGGGCTGGTTCAGA |
| | | | CAAGCCCCCGGCAAGGAGAGACAGTACGTGGGCACCT |
| | | | ACTGGAGCGGCTACGCCCACTACGGCGACTTCGAGAAC |
| | | | AACAGATTCACCATCAGCAGAGACAACAGCAAGAACA |
| | | | CCGTGTACCTGCAGATGAACAGCCTGAGAGCCGAGGAC |
| | | | ACCGCCGTGTACTACTGCGCCATCAGCCTGAAGTACCC |
| | | | CATGGCCACCGTGACCCCCGACTACCAAGGCCAAGGCA |
| | | | CCCTGGTGACCGTGAGCAGC |
| BGA-3726 | SEQ ID NO: 54 | HCDR1 | GRTFDNYA |
| (IMGT) | | (IMGT) | |
| | SEQ ID NO: 55 | HCDR2 | YWSGYA |
| | | (IMGT) | |
| | SEQ ID NO: 56 | HCDR3 | AISLKYPMATVTPDY |
| | | (IMGT) | |
| BGA-3726 | SEQ ID NO: 1 | HCDR1 | NYAVG |
| (Kabat) | | (Kabat) | |
| | SEQ ID NO: 2 | HCDR2 | TYWSGYAHYGDFENN |
| | | (Kabat) | |
| | SEQ ID NO: 3 | HCDR3 | SLKYPMATVTPDY |
| | | (Kabat) | |
| BGA-3726 | SEQ ID NO: 57 | HCDR1 | GRTFDNY |
| (Chothia) | | (Chothia) | |
| | SEQ ID NO: 58 | HCDR2 | WSGY |
| | | (Chothia) | |
| | SEQ ID NO: 59 | HCDR3 | SLKYPMATVTPDY |
| | | (Chothia) | |
| BGA-3726 | SEQ ID NO: 6 | VH AA | EVQLLESGGGLVQPGGSLRLSCAASGRTFDNYAVGWFRQ |
| | | | APGKEREYVGTYWSGYAHYGDFENNRFTISRDNSKNTVY |
| | | | LQMNSLRAEDTAVYYCAISLKYPMATVTPDYQGQGTLVT |
| | | | VSS |
| | SEQ ID NO: 7 | VH DNA | GAAGTGCAGCTGCTGGAGTCCGGGGGCGGCCTGGTCCA |
| | | | ACCCGGGGGCAGCCTGAGACTGAGCTGCGCCGCCTCCG |
| | | | GCAGAACCTTCGACAACTACGCCGTGGGCTGGTTCAGA |
| | | | CAAGCCCCCGGCAAGGAGAGAGAGTACGTGGGCACCT |
| | | | ACTGGAGCGGCTACGCCCACTACGGCGACTTCGAGAAC |
| | | | AACAGATTCACCATCAGCAGAGACAACAGCAAGAACA |
| | | | CCGTGTACCTGCAGATGAACAGCCTGAGAGCCGAGGAC |
| | | | ACCGCCGTGTACTACTGCGCCATCAGCCTGAAGTACCC |
| | | | CATGGCCACCGTGACCCCCGACTACCAAGGCCAAGGCA |
| | | | CCCTGGTGACCGTGAGCAGC |
| BGA-3544 | SEQ ID NO: 54 | HCDR1 | GRTFDNYA |
| (IMGT) | | (IMGT) | |
| | SEQ ID NO: 55 | HCDR2 | YWSGYA |
| | | (IMGT) | |
| | SEQ ID NO: 56 | HCDR3 | AISLKYPMATVTPDY |
| | | (IMGT) | |
| BGA-3544 | SEQ ID NO: 1 | HCDR1 | NYAVG |
| (Kabat) | | (Kabat) | |
| | SEQ ID NO: 10 | HCDR2 | TYWSGYAHYGDFEKG |
| | | (Kabat) | |
| | SEQ ID NO: 3 | HCDR3 | SLKYPMATVIPDY |
| | | (Kabat) | |
| BGA-3544 | SEQ ID NO: 57 | HCDR1 | GRTFDNY |
| (Chothia) | | (Chothia) | |
| | SEQ ID NO: 58 | HCDR2 | WSGY |
| | | (Chothia) | |
| | SEQ ID NO: 59 | HCDR3 | SLKYPMATVTPDY |
| | | (Chothia) | |
| BGA-3544 | SEQ ID NO: 11 | VH AA | EVQLVESGGGLVQPGGSLRLSCAASGRTFDNYAVGWFRQ |
| | | | APGKEREYVGTYWSGYAHYGDFEKGRFTISRDNSKNTVY |
| | | | LQMNNLRPEDTAVYYCAISLKYPMATVTPDYQGQGTQVT |
| | | | VSS |
| | SEQ ID NO: 12 | VH DNA | GAAGTGCAGCTGGTGGAGTCCGGGGGCGGCCTGGTCCA |
| | | | ACCCGGGGGCAGCCTGAGACTGAGCTGCGCCGCCTCCG |
| | | | GCAGAACCTTCGACAACTACGCCGTGGGCTGGTTCAGA |
| | | | CAAGCCCCCGGCAAGGAGAGAGAGTACGTGGGCACCT |
| | | | ACTGGAGCGGCTACGCCCACTACGGCGACTTCGAGAAG |
| | | | GGCAGATTCACCATCAGCAGAGACAACAGCAAGAACA |
| | | | CCGTGTACCTGCAGATGAACAACCTGAGACCCGAGGAC |
| | | | ACCGCCGTGTACTACTGCGCCATCAGCCTGAAGTACCC |
| | | | CATGGCCACCGTGACCCCCGACTACCAAGGCCAAGGCA |
| | | | CCCAAGTGACCGTGAGCAGC |
| BGA-9502 | SEQ ID NO: 54 | HCDR1 | GRTFDNYA |
| (IMGT) | | (IMGT) | |
| | SEQ ID NO: 55 | HCDR2 | YWSGYA |
| | | (IMGT) | |
| | SEQ ID NO: 56 | HCDR3 | AISLKYPMATVTPDY |
| | | (IMGT) | |
| BGA-9502 | SEQ ID NO: 1 | HCDR1 | NYAVG |
| (Kabat) | | (Kabat) | |
| | SEQ ID NO: 10 | HCDR2 | TYWSGYAHYGDFEKG |
| | | (Kabat) | |
| | SEQ ID NO: 3 | HCDR3 | SLKYPMATVTPDY |
| | | (Kabat) | |
| BGA-9502 | SEQ ID NO: 57 | HCDR1 | GRTFDNY |
| (Chothia) | | (Chothia) | |
| | SEQ ID NO: 58 | HCDR2 | WSGY |
| | | (Chothia) | |
| | SEQ ID NO: 59 | HCDR3 | SLKYPMATVTPDY |
| | | (Chothia) | |
| BGA-9502 | SEQ ID NO: 13 | VH AA | EVQLVESGGGLVQPGGSLRLSCAASGRTFDNYAVGWFRQ |
| | | | APGKEREYVGTYWSGYAHYGDFEKGRFTISRDNAKNTVY |
| | | | LQMNNLRPEDTAVYYCAISLKYPMATVTPDYQGQGTQVT |
| | | | VSS |
| | SEQ ID NO: 14 | VH DNA | GAAGTGCAGCTGGTGGAGTCCGGGGGCGGCCTGGTCCA |
| | | | ACCCGGGGGCAGCCTGAGACTGAGCTGCGCCGCCTCCG |
| | | | GCAGAACCTTCGACAACTACGCCGTGGGCTGGTTCAGA |
| | | | CAAGCCCCCGGCAAGGAGAGAGAGTACGTGGGCACCTA |
| | | | CTGGAGCGGCTACGCCCACTACGGCGACTTCGAGAAGG |
| | | | GCAGATTCACCATCAGCAGAGACAACGCCAAGAACACC |
| | | | GTGTACCTGCAGATGAACAACCTGAGACCCGAGGACAC |
| | | | CGCCGTGTACTACTGCGCCATCAGCCTGAAGTACCCCAT |
| | | | GGCCACCGTGACCCCCGACTACCAAGGCCAAGGCACCC |
| | | | AAGTGACCGTGAGCAGC |
| BGA-7031 | SEQ ID NO: 54 | HCDR1 | GRTFDNYA |
| (IMGT) | | (IMGT) | |
| | SEQ ID NO: 55 | HCDR2 | YWSGYA |
| | | (IMGT) | |
| | SEQ ID NO: 56 | HCDR3 | AISLKYPMATVTPDY |
| | | (IMGT) | |
| BGA-7031 | SEQ ID NO: 1 | HCDR1 | NYAVG |
| (Kabat) | | (Kabat) | |
| | SEQ ID NO: 2 | HCDR2 | TYWSGYAHYGDFENN |
| | | (Kabat) | |
| | SEQ ID NO: 3 | HCDR3 | SLKYPMATVTPDY |
| | | (Kabat) | |
| BGA-7031 | SEQ ID NO: 57 | HCDR1 | GRTFDNY |
| (Chothia) | | (Chothia) | |
| | SEQ ID NO: 58 | HCDR2 | WSGY |
| | | (Chothia) | |
| | SEQ ID NO: 59 | HCDR3 | SLKYPMATVTPDY |
| | | (Chothia) | |
| BGA-7031 | SEQ ID NO: 15 | VH AA | EVQLVESGGGLVQPGGSLRLSCAASGRTFDNYAVGWFRQ |
| | | | APGKEREYVGTYWSGYAHYGDFENNRFTISRDNSKNTVY |
| | | | LQMNNLRPEDTAVYYCAISLKYPMATVTPDYQGQGTQVT |
| | | | VSS |
| | SEQ ID NO: 16 | VH DNA | GAAGTGCAGCTGGTGGAGTCCGGGGGCGGCCTGGTCCA |
| | | | ACCCGGGGGCAGCCTGAGACTGAGCTGCGCCGCCTCCG |
| | | | GCAGAACCTTCGACAACTACGCCGTGGGCTGGTTCAGA |
| | | | CAAGCCCCCGGCAAGGAGAGAGAGTACGTGGGCACCTA |
| | | | CTGGAGCGGCTACGCCCACTACGGCGACTTCGAGAACA |
| | | | ACAGATTCACCATCAGCAGAGACAACAGCAAGAACACC |
| | | | GTGTACCTGCAGATGAACAACCTGAGACCCGAGGACAC |
| | | | CGCCGTGTACTACTGCGCCATCAGCCTGAAGTACCCCAT |
| | | | GGCCACCGTGACCCCCGACTACCAAGGCCAAGGCACCC |
| | | | AAGTGACCGTGAGCAGC |
| BGA-2524 | SEQ ID NO: 54 | HCDR1 | GRTFDNYA |
| (IMGT) | | (IMGT) | |
| | SEQ ID NO: 55 | HCDR2 | YWSGYA |
| | | (IMGT) | |
| | SEQ ID NO: 56 | HCDR3 | AISLKYPMATVTPDY |
| | | (IMGT) | |
| BGA-2524 | SEQ ID NO: 1 | HCDR1 | NYAVG |
| (Kabat) | | (Kabat) | |
| | SEQ ID NO: 2 | HCDR2 | TYWSGYAHYGDFENN |
| | | (Kabat) | |
| | SEQ ID NO: 3 | HCDR3 | SLKYPMATVTPDY |
| | | (Kabat) | |
| BGA-2524 | SEQ ID NO: 57 | HCDR1 | GRTFDNY |
| (Chothia) | | (Chothia) | |
| | SEQ ID NO: 58 | HCDR2 | WSGY |
| | | (Chothia) | |
| | SEQ ID NO: 59 | HCDR3 | SLKYPMATVTPDY |
| | | (Chothia) | |
| BGA-2524 | SEQ ID NO: 17 | VH AA | EVQLVESGGGLVQPGGSLRLSCAASGRTFDNYAVGWFRQ |
| | | | APGKEREYVGTYWSGYAHYGDFENNRFTISRDNAKNTVY |
| | | | LQMNNLRPEDTAVYYCAISLKYPMATVTPDYQGQGTQVT |
| | | | VSS |
| | SEQ ID NO: 18 | VH DNA | GAAGTGCAGCTGGTGGAGTCCGGGGGCGGCCTGGTCCA |
| | | | ACCCGGGGGCAGCCTGAGACTGAGCTGCGCCGCTAGCG |
| | | | GCAGAACCTTCGACAACTACGCCGTGGGCTGGTTCAGA |
| | | | CAAGCCCCCGGCAAGGAGAGAGAGTACGTGGGCACCTA |
| | | | CTGGAGCGGCTACGCCCACTACGGCGACTTCGAGAACA |
| | | | ACAGATTCACCATCAGCAGAGACAACGCCAAGAACACC |
| | | | GTGTACCTGCAGATGAACAACCTGAGACCCGAGGACAC |
| | | | CGCCGTGTACTACTGCGCCATCAGCCTGAAGTACCCCAT |
| | | | GGCCACCGTGACCCCCGACTACCAAGGCCAAGGCACCC |
| | | | AAGTGACCGTGAGCAGC |

The present disclosure provides for antibodies or antigen-binding fragments thereof that specifically bind to human CD137. Antibodies or antigen-binding fragments of the present disclosure include, but are not limited to, the antibodies or antigen-binding fragments thereof, generated as described below.

The present disclosure provides for antibody or antigen-binding fragment thereof which specifically binds human CD137, wherein the antibody or antigen-binding fragment thereof specifically binds to an epitope comprising, essentially consisting of, or consisting of amino acid residues Phe36, Asp38, Pro49, Pro50, Asn5l, Thr6l, Cys62, Asp63, Ile64, Gln67 of human CD137 (SEQ ID NO: 35), optionally the epitope is determined by X-ray diffraction.

The present disclosure provides for antibody or antigen-binding fragment thereof which specifically binds human CD137, wherein the antibody or antigen-binding fragment thereof specifically binds to an epitope comprising, essentially consisting of, or consisting of amino acid residues Ser55, Ala56, Arg75, Glu85, Ala97 and Gly98 of human CD137 (SEQ ID NO: 35), optionally the epitope is determined by X-ray diffraction.

The present disclosure provides for antibody or antigen-binding fragment thereof which specifically binds human CD137, wherein the antibody or antigen-binding fragment thereof specifically binds to a human CD137 dimer comprising, or consisting of a first human CD137 monomer and a second human CD137 monomer, wherein the antibody or antigen-binding fragment thereof specifically binds to an epitope of the first human CD137 monomer (SEQ ID NO:35) comprising, essentially consisting of or consisting of amino acid residues Phe36, Asp38, Pro49, Pro50, Asn51, Thr61, Cys62, Asp63, Ile64, Gln67, and the antibody or antigen-binding fragment thereof specifically binds to an epitope of the second human CD137 monomer (SEQ ID NO:35) comprising, essentially consisting of or consisting of amino acid residues Ser55, Ala56, Arg75, Glu85, Ala97 and Gly98, optionally the epitope is determined by X-ray diffraction, and optionally the antibody or antigen-binding fragment thereof binds to a human CD137 dimer and promotes human CD137 clustering.

The present disclosure provides for antibody or antigen-binding fragment thereof which specifically binds human CD137, wherein the antibody or antigen-binding fragment thereof specifically binds to an epitope comprising one or more amino acid residues selected from the group consisting of Phe36, Asp38, Pro49, Pro50, Asn51, Thr61, Cys62, Asp63, Ile64 and Gln67 of human CD137 (SEQ ID NO: 35).

The present disclosure provides for antibody or antigen-binding fragment thereof which specifically binds human CD137, wherein the antibody or antigen-binding fragment thereof specifically binds to an epitope comprising one or more amino acid residues selected from the group consisting of Ser55, Ala56, Arg75, Glu85, Ala97 and Gly98 of human CD137 (SEQ ID NO: 35).

The present disclosure provides for antibody or antigen-binding fragment thereof which specifically binds human CD137, wherein the antibody or antigen-binding fragment thereof specifically binds to a human CD137 dimer comprising, or consisting of a first human CD137 monomer and a second human CD137 monomer, wherein the antibody or antigen-binding fragment thereof specifically binds to an epitope of the first human CD137 monomer (SEQ ID NO: 35) comprising one or more amino acid residues selected from the group consisting of Phe36, Asp38, Pro49, Pro50, Asn51, Thr61, Cys62, Asp63, Ile64, Gln67, and the antibody or antigen-binding fragment thereof specifically binds to an epitope of the second human CD137 monomer (SEQ ID NO: 35) comprising one or more amino acid residues selected from the group consisting of Ser55, Ala56, Arg75, Glu85, Ala97 and Gly98, optionally the antibody or antigen-binding fragment thereof binds to a human CD137 dimer and promotes human CD137 clustering.

The present disclosure provides for antibody or antigen-binding fragment thereof which specifically binds human CD137, wherein the antibody or antigen-binding fragment thereof specifically binds to an epitope comprising one or more amino acid residues selected from the group consisting of Phe36, Asp38, Pro49, Pro50, Asn51, Thr61, Cys62, Asp63, Ile64, Gln67, Ser55, Ala56, Arg75, Glu85, Ala97 and Gly98 of human CD137 (SEQ ID NO: 35).

The present disclosure provides for antibody or antigen-binding fragment thereof which specifically binds human CD137, wherein the antibody or antigen-binding fragment thereof specifically binds to an epitope consisting of amino acid residues Phe36, Asp38, Pro49, Pro50, Asn51, Thr61, Cys62, Asp63, Ile64, Gln67 of human CD137 (SEQ ID NO: 35).

The present disclosure provides for antibody or antigen-binding fragment thereof which specifically binds human CD137, wherein the antibody or antigen-binding fragment thereof specifically binds to an epitope consisting of amino acid residues Ser55, Ala56, Arg75, Glu85, Ala97 and Gly98 of human CD137 (SEQ ID NO: 35).

The present disclosure provides for antibody or antigen-binding fragment thereof which specifically binds human CD137, wherein the antibody or antigen-binding fragment thereof specifically binds to a human CD137 dimer comprising, or consisting of a first human CD137 monomer and a second human CD137 monomer, wherein the antibody or antigen-binding fragment thereof specifically binds to an epitope of the first human CD137 (SEQ ID NO: 35) monomer consisting of amino acid residues Phe36, Asp38, Pro49, Pro50, Asn51, Thr61, Cys62, Asp63, Ile64, Gln67, and the antibody or antigen-binding fragment thereof specifically binds to an epitope of the second human CD137 monomer (SEQ ID NO: 35) consisting of amino acid residues Ser55, Ala56, Arg75, Glu85, Ala97 and Gly98, wherein the antibody or antigen-binding fragment thereof binds to a human CD137 dimer and promotes human CD137 clustering.

The present disclosure provides for antibody or antigen-binding fragment thereof which specifically binds human CD137, wherein the antibody or antigen-binding fragment thereof comprises a paratope comprising one or more amino acid residues selected from the group consisting of Asn31, Tyr32, Ala33, Trp52, Ser53, Tyr55, His57, Leu98, Lys99, Tyr100, Pro101, Thr104, Thr106, Tyr109 (natural order of sequence) or Asn31, Tyr32, Ala33, Trp52, Ser54, Tyr56, His58, Leu96, Lys97, Tyr98, Pro99, Thr100B, Thr100D, Tyr102 (Kabat nomenclature).

In one embodiment, the antibody or antigen-binding fragment thereof specifically binds human CD137 mainly binds the side surface of (e.g., human) CD137 CRD2 domain with CDR residues (e.g., Asn31, Tyr32, Ala33, Trp52, Ser54, Tyr56, His58, Leu96, Lys97, Tyr98, Pro99, Thr100B, Thr100D, Tyr102 (Kabat nomenclature) of human CD137 VHH).

In some embodiments, the epitope of human CD137, bound by the antibody or antigen-binding fragment thereof specifically binds human CD137 of the present disclosure, is determined by X-ray diffraction.

The present disclosure provides for antibodies or antigen-binding fragments that specifically bind to human CD137, wherein said antibodies or antibody fragments (e.g., antigen-binding fragments) comprise a VH domain having an amino acid sequence of SEQ ID NO: 4, SEQ ID NO: 8, SEQ ID NO: 6, SEQ ID NO: 11, SEQ ID NO: 13, SEQ ID NO: 15 or SEQ ID NO: 17 (Table 1). The present disclosure also provides antibodies or antigen-binding fragments that specifically bind human CD137, wherein said antibodies or antigen-binding fragments comprise a HCDR having an amino acid sequence of any one of the HCDRs listed in Table 1.

In one aspect, the present disclosure provides antibodies or antigen-binding fragments that specifically bind to human CD137, wherein said antibodies comprise (or alternatively, consist of) one, two, three, or more HCDRs having an amino acid sequence of any of the HCDRs listed in Table 1.

In one embodiment, the antibody or an antigen-binding fragment thereof comprises one or more complementarity determining regions (CDRs) comprising an amino acid sequence selected from SEQ ID NO: 1, SEQ ID NO: 2, SEQ ID NO: 3, SEQ ID NO: 10; or selected from SEQ ID NO: 54, SEQ ID NO: 55, SEQ ID NO: 56; or selected from SEQ ID NO: 57, SEQ ID NO: 58, SEQ ID NO: 59.

In one embodiment, the anti-CD137 antibodies or antigen-binding fragments thereof comprise: (i) a HCDR1 (Heavy Chain Complementarity Determining Region 1), a HCDR2 and a HCDR3 from the heavy chain variable region (VH) set forth in SEQ ID NO: 4; (ii) a HCDR1, a HCDR2 and a HCDR3 from the heavy chain variable region (VH) set forth in SEQ ID NO: 8; (iii) a HCDR1, a HCDR2 and a HCDR3 from the heavy chain variable region (VH) set forth in SEQ ID NO: 6; (iv) a HCDR1, a HCDR2 and a HCDR3 from the heavy chain variable region (VH) set forth in SEQ ID NO: 11; (v). a HCDR1, a HCDR2 and a HCDR3 from the heavy chain variable region (VH) set forth in SEQ ID NO: 13; (vi) a HCDR1, a HCDR2 and a HCDR3 from the heavy chain variable region (VH) set forth in SEQ ID NO: 15; or (vii) a HCDR1, a HCDR2 and a HCDR3 from the heavy chain variable region (VH) set forth in SEQ ID NO: 17.

In one embodiment, the anti-CD137 antibodies or antigen-binding fragments thereof comprise: (i) a heavy chain variable region (VH) that comprises (a) a HCDR1 of SEQ ID NO: 1, (b) a HCDR2 of SEQ ID NO: 2, and (c) a HCDR3 of SEQ ID NO: 3; or (ii) a heavy chain variable region (VH) that comprises (a) a HCDR1 of SEQ ID NO: 1, (b) a HCDR2 of SEQ ID NO: 10, and (c) a HCDR3 of SEQ ID NO: 3, according to the Kabat numbering.

Other antibodies or antigen-binding fragments thereof of the present disclosure include amino acids that have been changed, yet have at least 60%, 70%, 80%, 90%, 91%, 92%, 93%, 94%, 95% 96%, 97%, 98%, or 99% percent identity in the CDR regions with the CDR regions disclosed in Table 1. In some aspects, it includes amino acid changes (insertion, deletion or substitution, optionally conservative amino acid substitutions) wherein no more than 1, 2, 3, 4 or 5 amino acids have been changed in the CDR regions when compared with the CDR regions depicted in the sequence described in Table 1, while maintaining binding specificity and affinity.

Other antibodies of the present disclosure include those where the amino acids or nucleic acids encoding the amino acids have been changed; yet have at least 60, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% percent identity to the sequences described in Table 1, optionally the corresponding sequences of CDRs do not change. In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions (e.g., the frameworks regions of the variable regions) when compared with the variable regions depicted in the sequence described in Table 1, while retaining therapeutic activity/binding specificity/affinity, optionally the corresponding sequences of CDRs do not change. In some aspects, it includes changes in the amino acid sequences wherein 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed, for example, inserted, deleted, or substituted (optionally conservative amino acid substitutions) in the variable regions (e.g., the frameworks regions of the variable regions) when compared with the variable regions depicted in the sequence described in Table 1, while retaining therapeutic activity/binding specificity/affinity, optionally the corresponding sequences of CDRs do not change.

In some embodiments, the present disclosure provides for antibodies or antigen-binding fragments thereof that specifically bind to human CD137 comprising a heavy chain variable region (VH) that comprises (a) a HCDR1 of SEQ ID NO: 1, (b) a HCDR2 of SEQ ID NO: 2, and (c) a HCDR3 of SEQ ID NO: 3; wherein the amino acids F37, Y47, G49, and 194 (Kabat numbering) in the framework region are retained.

In some embodiments, the present disclosure provides for antibodies or antigen-binding fragments thereof that specifically bind to human CD137 comprising a heavy chain variable region (VH) that comprises (a) a HCDR1 of SEQ ID NO: 1, (b) a HCDR2 of SEQ ID NO: 10, and (c) a HCDR3 of SEQ ID NO: 3; wherein the amino acids F37, Y47, G49, and 194 (Kabat numbering) in the framework region are retained.

In some embodiments, the present disclosure provides for antibodies or antigen-binding fragments thereof that specifically bind to human CD137 comprising a VH domain having an amino acid sequence described in Table 1 or the variant thereof, wherein the HCDR1, HCDR2 and HCDR3 are not changed and amino acids F37, Y47, G49, and 194 (Kabat numbering) in the framework region are retained.

In some embodiments, the present disclosure provides for antibodies or antigen-binding fragments thereof that specifically bind to human CD137 comprising a heavy chain variable region comprising an amino acid sequence at least 90, 91, 92, 93, 94, 95, 96, 97, 98 or 99% identical to SEQ ID NO: 4, SEQ ID NO: 8, SEQ ID NO: 6, SEQ ID NO: 11, SEQ ID NO: 13, SEQ ID NO: 15 or SEQ ID NO: 17, wherein the HCDR1, HCDR2 and HCDR3 are not changed and amino acids F37, Y47, G49, and 194 (Kabat numbering) in the framework region are retained.

In another embodiment, the present disclosure provides for antibodies or antigen-binding fragments thereof that specifically bind to human CD137 with a binding affinity (K_{D}) of from 1×10⁻⁶ M to 1×10⁻¹⁰ M. In another embodiment, the anti-CD137 antibodies or antigen-binding fragments thereof bind to human CD137 with a binding affinity (K_{D}) of about 1×10⁻⁶ M, about 1×10⁻⁷ M, about 1×10⁻⁸ M, about 1×10⁻⁹ M or about 1×10⁻¹⁰ M.

The present disclosure also provides nucleic acid sequences that encode VH and the full length heavy chain of the antibodies that specifically bind to human CD137. Such nucleic acid sequences can be optimized for expression in mammalian cells.

The present disclosure also provides for antibodies and antigen-binding fragments thereof that bind to the same epitope as do the anti-CD137 antibodies described in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CD137 demonstrates that the test antibody can compete with that antibody or antigen-binding fragments thereof for binding to CD137. Such an antibody can, without being bound to any one theory, bind to the same or a related (e.g., a structurally similar or spatially proximal) epitope on CD137 as the antibody or antigen-binding fragments thereof with which it competes. In a certain aspect, the antibody that binds to the same epitope on CD137 as the antibodies or antigen-binding fragments thereof of the present disclosure is a human or humanized monoclonal antibody. Such human or humanized monoclonal antibodies can be prepared and isolated as described herein.

In some embodiments, the anti-CD137 antibodies comprise at least one antigen-binding site, at least a variable region. In some embodiments, the anti-CD137 antibodies comprise an antigen-binding fragment from an CD137 antibody described herein. In some embodiments, the anti-CD137 antibody is isolated or recombinant. In some embodiments, the anti-CD137 antibodies also encompass multispecific antibodies targeting CD137 as at least one arm and targeting other antigen(s) as another arm(s).

### III. Anti-CD137 Multispecific Antibodies

In one embodiment, the anti-CD137 antibodies as disclosed herein can be used to construct multispecific antibodies with other modalities such as human tumor associated antigen (TAA), immune checkpoints or immune stimulators.

In one embodiment, the anti-CD137 antibodies as disclosed herein can be incorporated into an anti-CD137×TAA multispecific antibody, wherein TAA is an antibody or fragment thereof directed to any human tumor associated antigen. An antibody molecule is a multispecific antibody molecule, for example, it comprises a number of antigen binding domains, wherein at least one antigen binding domain sequence specifically binds a human TAA as a first antigen/epitope and a second antigen binding domain sequence specifically binds human CD137 as a second antigen/epitope. In one embodiment, the multispecific antibody comprises a third, fourth or fifth antigen binding domain. In one embodiment, the multispecific antibody is a bispecific antibody, a trispecific antibody, or tetraspecific antibody. In each example, the multispecific antibody comprises at least one anti-TAA antigen binding domain and at least one anti-CD137 antigen binding domain.

In one embodiment, the multispecific antibody is a bispecific antibody. As used herein, a bispecific antibody specifically binds only two antigens. The bispecific antibody comprises a first antigen binding domain which specifically binds TAA and a second antigen binding domain that specifically binds human CD137. This includes a bispecific antibody comprising a heavy chain variable domain and a light chain variable domain which specifically binds TAA and a heavy chain variable domain which specifically binds human CD137. In some embodiments, the bispecific antibody comprises antigen binding fragments, wherein the antigen-binding fragment can be a Fab, F(ab′)₂, Fv, a single chain Fv (scFv), or a single domain antibody.

In some embodiments, the second antigen binding domain that specifically binds to human CD137 include the anti-CD137 antibodies disclosed in Section II.

In one embodiment, the multispecific antibody of the present disclosure binds to human TAA and/or human CD137 with a binding affinity (K_{D}) of from 1×10⁻⁶ M to 1×10⁻¹⁰ M. In another embodiment, the multispecific antibody of the present disclosure binds to human TAA and/or human CD137 with a binding affinity (K_{D}) of about 1×10⁻⁶ M, about 1×10⁻⁷ M, about 1×10⁻⁸ M, about 1×10⁻⁹ M or about 1×10⁻¹⁰ M.

In one embodiment, the present disclosure provides for a multispecific antibody or antigen-binding fragment thereof, wherein the first antigen binding domain specifically binds to human TAA, and the second antigen binding domain that specifically binds to human CD137 comprises: (i) a heavy chain variable region (VH) that comprises (a) a HCDR1 of SEQ ID NO: 1, (b) a HCDR2 of SEQ ID NO: 2, (c) a HCDR3 of SEQ ID NO: 3; or (ii) a heavy chain variable region (VH) that comprises (a) a HCDR1 of SEQ ID NO: 1, (b) a HCDR2 of SEQ ID NO: 10, (c) a HCDR3 of SEQ ID NO: 3.

In another embodiment, the present disclosure provides for a multispecific antibody or antigen-binding fragment thereof, wherein the first antigen binding domain specifically binds to human TAA, and the second antigen binding domain that specifically binds to human CD137 comprises: (i) a heavy chain variable region (VH) comprising an amino acid sequence at least 90, 91, 92, 93, 94, 95, 96, 97, 98 or 99% identical to SEQ ID NO: 17; (ii) a heavy chain variable region (VH) comprising an amino acid sequence at least 90, 91, 92, 93, 94, 95, 96, 97, 98 or 99% identical to SEQ ID NO: 11; (iii) a heavy chain variable region (VH) comprising an amino acid sequence at least 90, 91, 92, 93, 94, 95, 96, 97, 98 or 99% identical to SEQ ID NO: 13; (iv) a heavy chain variable region (VH) comprising an amino acid sequence at least 90, 91, 92, 93, 94, 95, 96, 97, 98 or 99% identical to SEQ ID NO: 15; or (v) a heavy chain variable region (VH) comprising an amino acid sequence at least 90, 91, 92, 93, 94, 95, 96, 97, 98 or 99% identical to SEQ ID NO: 4.

In another embodiment, the present disclosure provides for a multispecific antibody or antigen-binding fragment thereof, wherein the first antigen binding domain specifically binds to human TAA, and the second antigen binding domain that specifically binds to human CD137 comprises: (i) a heavy chain variable region (VH) that comprises SEQ ID NO: 4; (ii) a heavy chain variable region (VH) that comprises SEQ ID NO: 11; (iii) a heavy chain variable region (VH) that comprises SEQ ID NO: 13; (iv) a heavy chain variable region (VH) that comprises SEQ ID NO: 15; or (v) a heavy chain variable region (VH) that comprises SEQ ID NO: 17.

In one embodiment, the TAA is human GPC3. The first antigen binding domain that specifically binds to human GPC3 include the anti-GPC3 antibodies disclosed in Section I.

The present disclosure provides multivalent antibodies (e.g., tetravalent antibodies) with at least two antigen binding domains, which can be readily produced by recombinant expression of nucleic acid encoding the polypeptide chains of the antibody. The multivalent antibody herein comprises three to eight, but preferably four, antigen binding domains, which specifically bind at least two antigens.

### IV. Anti-GPC3×CD137 Multispecific Antibodies

In one embodiment, the anti-GPC3 and anti-CD137 antibodies as disclosed herein can be incorporated into an anti-GPC3×CD137 multispecific antibody. An antibody molecule is a multispecific antibody molecule, for example, it comprises a number of antigen binding domains, wherein at least one antigen binding domain sequence specifically binds GPC3 as a first epitope/antigen and a second antigen binding domain sequence specifically binds CD137 as a second epitope/antigen. In one embodiment, the multispecific antibody comprises a third, fourth or fifth antigen binding domain. In one embodiment, the multispecific antibody is a bispecific antibody, a trispecific antibody, or tetraspecific antibody. In each example, the multispecific antibody comprises at least one anti-GPC3 antigen binding domain and at least one anti-CD137 antigen binding domain.

In one embodiment, the multispecific antibody is a bispecific antibody. As used herein, a bispecific antibody specifically binds only two antigens. The bispecific antibody comprises a first antigen binding domain which specifically binds human GPC3 and a second antigen binding domain that specifically binds human CD137. This includes a bispecific antibody comprising a heavy chain variable domain and a light chain variable domain which specifically binds human GPC3 as a first epitope/antigen and a heavy chain variable domain which specifically binds human CD137 as a second epitope/antigen. In some embodiments, the bispecific antibody comprises antigen binding fragments, wherein the antigen-binding fragment can be a Fab, F(ab′)₂, Fv, a single chain Fv (scFv), or a single domain antibody.

The present disclosure provides for a multispecific antibody or antigen-binding fragment thereof, comprising a first antigen binding domain that specifically binds to human Glypican 3 (GPC3) and a second antigen binding domain that specifically binds to human CD137.

The first antigen binding domain that specifically binds to human Glypican 3 (GPC3) includes the anti-GPC3 antibodies described in Section I. The second antigen binding domain that specifically binds to human CD137 include the anti-CD137 antibodies disclosed in Section II.

In one embodiment, the multispecific antibody of the present disclosure binds to human GPC3 and/or human CD137 with a binding affinity (K_{D}) of from 1×10⁻⁶ M to 1×10⁻¹⁰ M. In another embodiment, the multispecific antibody of the present disclosure binds to human GPC3 and/or human CD137 with a binding affinity (K_{D}) of about 1×10⁻⁶ M, about 1×10⁻⁷ M, about 1×10⁻⁸ M, about 1×10⁻⁹ M or about 1×10⁻¹⁰ M.

In one embodiment, the multispecific antibody of the present disclosure has specific binding to human GPC3 and shows high affinity to both human GPC3 and monkey GPC3. In another embodiment, the multispecific antibody of the present disclosure has specific binding to human CD137. In another embodiment, the multispecific antibody of the present disclosure shows high affinity to both human CD137 and monkey CD137.

In one embodiment, the present disclosure provides for a multispecific antibody or antigen-binding fragment thereof, wherein the first antigen binding domain that specifically binds to human GPC3 comprises: a heavy chain variable region (VH) that comprises (a) a HCDR1 of SEQ ID NO: 45, (b) a HCDR2 of SEQ ID NO: 46, (c) a HCDR3 of SEQ ID NO: 47; and a light chain variable region (VL) that comprises (d) a LCDR1 of SEQ ID NO: 48, (e) a LCDR2 of SEQ ID NO: 49, (f) a LCDR3 of SEQ ID NO: 50, according to the Kabat numbering; and wherein the second antigen binding domain that specifically binds to human CD137 comprises: (i) a heavy chain variable region (VH) that comprises (a) a HCDR1 of SEQ ID NO: 1, (b) a HCDR2 of SEQ ID NO: 2, (c) a HCDR3 of SEQ ID NO: 3; or (ii) a heavy chain variable region (VH) that comprises (a) a HCDR1 of SEQ ID NO: 1, (b) a HCDR2 of SEQ ID NO: 10, (c) a HCDR3 of SEQ ID NO: 3, according to the Kabat numbering.

In another embodiment, the present disclosure provides for a multispecific antibody or antigen-binding fragment thereof, wherein the first antigen binding domain that specifically binds to human GPC3 comprises: a heavy chain variable region (VH) that comprises SEQ ID NO: 41, and a light chain variable region (VL) that comprises SEQ ID NO: 43; and wherein the second antigen binding domain that specifically binds to human CD137 comprises: (i) a heavy chain variable region (VH) that comprises SEQ ID NO: 4; (ii) a heavy chain variable region (VH) that comprises SEQ ID NO: 11; (iii) a heavy chain variable region (VH) that comprises SEQ ID NO: 13; (iv) a heavy chain variable region (VH) that comprises SEQ ID NO: 15; or (v) a heavy chain variable region (VH) that comprises SEQ ID NO: 17.

In another embodiment, the present disclosure provides a multispecific antibody or antigen-binding fragment thereof, wherein the multispecific antibody or antigen-binding fragment is (i) BE-933 comprising the first polypeptide of SEQ ID NO: 27 and the second polypeptide of SEQ ID NO: 23; (ii) BE-774 comprising the first polypeptide of SEQ ID NO: 25 and the second polypeptide of SEQ ID NO: 23; (iii) BE-653 comprising the first polypeptide of SEQ ID NO: 29 and the second polypeptide of SEQ ID NO: 23; (iv) BE-915 comprising the first polypeptide of SEQ ID NO: 21 and the second polypeptide of SEQ ID NO: 23; (v) BE-647 comprising the first polypeptide of SEQ ID NO: 31 and the second polypeptide of SEQ ID NO: 23; or (vi) BE-621 comprising the first polypeptide of SEQ ID NO: 33 and the second polypeptide of SEQ ID NO: 23.

Other multispecific antibody or antigen-binding fragment thereof of the present disclosure include those where the amino acids or nucleic acids encoding the amino acids have been changed; yet have at least 60, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% percent identity to the sequences described herein (e.g., CDRs do not change). In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions (e.g., framework regions) when compared with the variable regions described herein, while retaining therapeutic activity/binding specificity/affinity.

### V. Others

### Format and Module Ratios

In one embodiment, the anti-CD137 antibodies as disclosed herein can be used to construct multispecific antibodies with other modalities such as TAA, immune checkpoints or immune stimulators. TAA, (e.g., GPC3) is used as an example below for format and ratio description. The description for GPC3 in the below embodiments could also apply for other TAA.

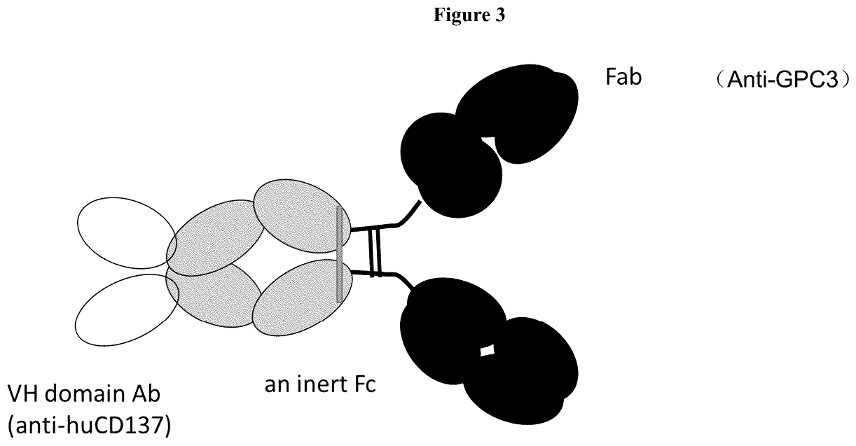

The multispecific antibodies of the disclosure could be in different formats. In one embodiment, the multispecific antibodies of the disclosure have the format as disclosed below, including: (1) the format A provides a symmetric IgG-like multispecific molecule with Fab×VH configuration. Anti-huCD137 VH domain antibody was fused to the C-termini of Fc (CH3 domain) of an anti-GPC3 antibody with a linker in between as shown in FIG. 3; (2) the format B also provides a symmetric IgG-like multispecific molecule with Fab×VH configuration. Anti-huCD137 VH domain antibody was fused to the C-termini of light chain (Cκ) of an anti-GPC3 antibody with a linker in between; (3) the format C provides a symmetric VH antibody-like multispecific molecule with Fab×VH configuration. The Fab region of an anti-GPC3 antibody was fused to the N-termini of VH of anti-huCD137 VH domain Ab with a linker in between; and (4) the format D also provides a symmetric IgG-like multispecific molecule with Fab×VH configuration. Anti-huCD137 VH domain antibody was fused to the N-termini of heavy chain (VH) of an anti-GPC3 antibody with a linker in between. In one embodiment, the multispecific antibodies are in the format A as shown in FIG. 3.

The multispecific antibodies of the disclosure can be constructed with different module ratios such as 1:1. In one embodiment, an inert Fc can be used for multispecific antibodies and the Azymetric™ Platform from Zymeworks can be utilized to assemble the FabXVH configuration, in which ZW1 mutations (chain A: T350V/L351Y/F405A/Y407V; chain B: T350V/T366L/K392L/T394W) can be introduced in the CH3 domain of heavy chain to allow efficient heterodimer formation (Von Kreudenstein et al., (2013) Mabs 5(5):646-54, incorporated by reference in its entirety). In one aspect, the specific ratio activates CD137 in a GPC3 dependent manner, and without the activation of CD137 in the absence of GPC3.

In one embodiment, the multispecific antibody or antigen-binding fragment thereof comprises: a) a first polypeptide comprising from N terminal to C terminal: a first heavy chain variable region (such as one first heavy chain variable region); a CH1 domain, a Fc domain, and a second heavy chain variable region (such as one second heavy chain variable region); optionally, C terminal of the Fc domain is linked to N terminal of the second heavy chain variable region via a linker; and b) a second polypeptide comprising from N terminal to C terminal: a first light chain variable region (such as one first light chain variable region); and a first light chain constant region; wherein the first heavy chain variable region and the first light chain variable region form the first antigen binding domain that specifically binds to human GPC3, and the second heavy chain variable region forms the second antigen binding domain that specifically binds to human CD137. In another embodiment, the multispecific antibody or antigen-binding fragment thereof comprises two of the first polypeptides and two of the second polypeptides.

### Linkers

It is also understood that the domains and/or regions of the polypeptide chains of the bispecific antibody can be separated by linker regions of various lengths. In some embodiments, the antigen binding domains are separated from each other, a CL, CH1, hinge, CH2, CH3, or the entire Fc region by a linker region. For example, VL1-CL-(linker) VH2-CH1. Such linker region may comprise a random assortment of amino acids, or a restricted set of amino acids. Such linker regions can be flexible or rigid (see e.g., US2009/0155275, incorporated by reference in its entirety).

Multispecific antibodies have been constructed by genetically fusing two single chain Fv (scFv) or Fab fragments with or without the use of flexible linkers (Mallender et al., J. Biol. Chem. 1994; 269:199-206; Mack et. al.., Proc. Natl. Acad. Sci. USA. 1995; 92:7021-5; Zapata et al., Protein Eng. 1995; 8.1057-62), via a dimerization device such as leucine Zipper (Kostelny et al., J. Immunol. 1992; 148:1547-53; de Kruifetal J. Biol. Chem. 1996; 271:7630-4) and Ig C/CH1 domains (Muller et al., FEBS Lett. 1998; 422:259-64); by diabody (Holliger et al., Proc. Nat. Acad. Sci. USA. 1993; 90:6444-8; Zhu et al., Bio/Technology (NY) 1996; 14:192-6); Fab-scFv fusion (Schoonjans et al., J. Immunol. 2000; 165:7050-7); and mini antibody formats (Pack et. al.., Biochemistry 1992; 31:1579-84; Pack et. al.., Bio/Technology 1993; 11:1271-7). Each reference mentioned in this paragraph incorporated by reference in their entirety.

The multispecific antibodies as disclosed herein comprise a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its antigen binding domains, CL domains, CH1 domains, Hinge region, CH2 domains, CH3 domains, or Fc regions. In some embodiments, the amino acids glycine and serine are comprised within the linker region. In another embodiment, the linker can be GS (SEQ ID NO: 97), GGS (SEQ ID NO: 98), GSG (SEQ ID NO: 99), SGG (SEQ ID NO: 100), GGG (SEQ ID NO: 101), GGGS (SEQ ID NO: 60), SGGG (SEQ ID NO: 61), GGGGS (SEQ ID NO: 62), GGGGSGS (SEQ ID NO: 63), GGGGSGS (SEQ ID NO: 64), GGGGSGGS (SEQ ID NO: 65), GGGGSGGGGS (SEQ ID NO: 66), GGGGSGGGGSGGGGS (SEQ ID NO: 67), AKTTPKLEEGEFSEAR (SEQ ID NO: 68), AKTTPKLEEGEFSEARV (SEQ ID NO: 69), AKTTPKLGG (SEQ ID NO: 70), SAKTTPKLGG (SEQ ID NO: 71), AKTTPKLEEGEFSEARV (SEQ ID NO: 72), SAKTTP (SEQ ID NO: 73), SAKTTPKLGG (SEQ ID NO: 74), RADAAP (SEQ ID NO: 75), RADAAPTVS (SEQ ID NO: 76), RADAAAAGGPGS (SEQ ID NO: 77), RADAAAA(G₄S)₄ (SEQ ID NO: 78), SAKTTP (SEQ ID NO: 79), SAKTTPKLGG (SEQ ID NO: 80), SAKTTPKLEEGEFSEARV (SEQ ID NO: 81), ADAAP (SEQ ID NO: 82), ADAAPTVSIFPP (SEQ ID NO: 83), TVAAP (SEQ ID NO: 84), TVAAPSVFIFPP (SEQ ID NO: 85), QPKAAP (SEQ ID NO: 86), QPKAAPSVTLFPP (SEQ ID NO: 87), AKTTPP (SEQ ID NO: 88), AKTTPPSVTPLAP (SEQ ID NO: 89), AKTTAP (SEQ ID NO: 90), AKTTAPSVYPLAP (SEQ ID NO: 91), ASTKGP (SEQ ID NO: 92), ASTKGPSVFPLAP (SEQ ID NO: 93), GENKVEYAPALMALS (SEQ ID NO: 94), GPAKELTPLKEAKVS (SEQ ID NO: 95), and GHEAAAVMQVQYPAS (SEQ ID NO: 96) or any combination thereof (see WO2007/024715, incorporated by reference in its entirety).

### Dimerization Specific Amino Acids

In one embodiment, the multivalent antibody comprises at least one dimerization specific amino acid change. The dimerization specific amino acid changes result in “knobs into holes” interactions, and increases the assembly of correct multivalent antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 family and WO2017059551, each incorporated by reference in their entirety. The dimerization specific amino acids can also be within the Fc domain and can be in combination with dimerization specific amino acids within the CH1 or CL domains. In one embodiment, the disclosure provides a bispecific antibody comprising at least one dimerization specific amino acid pair.

### Alteration of the Fe Region

The Fc region, if present, could be wild type Fc region of the subclass of IgG1, IgG2, IgG3, or IgG4.

In one embodiment, the antibody, multispecific antibody or antigen-binding fragment thereof comprises a Fc domain of IgG1 or IgG4 with reduced effector function. In another embodiment, the Fc domain comprises an amino acid sequence of SEQ ID NO: 53 or SEQ ID NO: 19. In another embodiment, the IgG1 Fc comprises mutations E233P, L234A, L235A, G236del, and P329A.

In one embodiment, the multispecific antibody or antigen-binding fragment thereof comprises a Fc domain with extended half-life. In another embodiment, the multispecific antibody or antigen-binding fragment thereof comprises a Fc domain of IgG1, wherein YTE mutation (M252Y/S254T/T256E, EU numbering, as described in U.S. Pat. No. 7,658,921, incorporated by reference in its entirety) located at CH2 of IgG Fc region were introduced.

In one embodiment, the multispecific antibody or antigen-binding fragment thereof comprises a Fc domain with reduced effector function and extended half-life. In another embodiment, the Fc domain comprises an amino acid sequence of SEQ ID NO: 20.

In another embodiment, antibodies of the present disclosure have strong Fc-mediated effector functions, and the antibodies mediate antibody-dependent cellular cytotoxicity (ADCC) against target cells expressing TAA (e.g., GPC3).

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al, each incorporated by reference in their entirety.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al., incorporated by reference in its entirety.

In yet another aspect, one or more amino acid residues are changed to thereby alter the ability of the antibody to fix complement. This approach is described in, e.g., the publication WO 94/29351 by Bodmer et al., incorporated by reference in its entirety. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., Mabs. 2009; 1:332-338, incorporated by reference in its entirety.

In another aspect, the Fc region is modified to increase the ability of the antibody to mediate antibody dependent cellular cytotoxicity (ADCC) and/or to increase the affinity of the antibody for an Fc receptor by modifying one or more amino acids. This approach is described in, e.g., the publication WO00/42072 by Presta, incorporated by reference in its entirety. Moreover, the binding sites on human IgG1 for FcγRI, FcγRII, FcγRIII and FcRn have been mapped and variants with improved binding have been described (see, Shields et al., J. Biol. Chem. 2001; 276:6591-6604), incorporated by reference in its entirety.

In still another aspect, the glycosylation of the multispecific antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al., each incorporated by reference in their entirety. Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., incorporated by reference in its entirety, describes a cell line with a functionally disrupted FUT8 gene, which encodes a fucosyl transferase, such that antibodies expressed in such a cell line exhibit hypofucosylation. Publication WO 03/035835 by Presta, incorporated by reference in its entirety, describes a variant CHO cell line, Lecl3 cells, with reduced ability to attach fucose to Asn (297)-linked carbohydrates, also resulting in hypofucosylation of antibodies expressed in that host cell (see also Shields et al., J. Biol. Chem. 2002; 277:26733-26740, incorporated by reference in its entirety). WO99/54342 by Umana et al., incorporated by reference in its entirety, describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 1999; 17:176-180, incorporated by reference in its entirety).

In another aspect, if a reduction of ADCC is desired, human antibody subclass IgG4 was shown in many previous reports to have only modest ADCC and almost no CDC effector function (Moore G L, et al., MAbs. 2010; 2:181-189, incorporated by reference in its entirety). However, natural IgG4 was found less stable in stress conditions such as in acidic buffer or under increasing temperature (Angal, S. Mol Immunol. 1993; 30:105-108; Dall'Acqua, W. et al., 1998 Biochemistry, 37:9266-9273; Aalberse et al., Immunol. 2002; 105:9-19, each incorporated by reference in their entirety). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., Science. 2007; 317:1554-157, incorporated by reference in its entirety). The mutation of serine to proline at position 228 (EU numbering system) appeared inhibitory to the IgG4 heavy chain separation (Angal, S. Mol Immunol. 1993; 30:105-108, incorporated by reference in its entirety; Aalberse et al., Immunol. 2002; 105:9-19, incorporated by reference in its entirety). Some of the amino acid residues in the hinge and 7Fc region were reported to have impact on antibody interaction with Fcγ receptors (Chappel S M, et al., Proc. Natl. Acad. Sci. USA. 1991; 88:9036-9040; Mukherjee, J. et al., FASEB J. 1995; 9:115-119; Armour, K. L. et al., Eur J Immunol. 1999; 29:2613-2624; Clynes, R. A. et al, 2000 Nature Medicine, 6:443-446; Arnold J. N., Annu Rev Immunol. 2007; 25:21-50, each incorporated by reference in their entirety). Furthermore, some rarely occurring IgG4 isoforms in human population can also elicit different physicochemical properties (Brusco, A. et al., Eur J Immunogenet. 1998; 25:349-55; Aalberse et al., Immunol. 2002; 105:9-19, each incorporated by reference in their entirety). To generate multispecific antibodies with low ADCC and CDC but with good stability, it is possible to modify the hinge and Fc region of human IgG4 and introduce a number of alterations. These modified IgG4 Fc molecules can be found in SEQ ID NOs: 83-88 of U.S. Pat. No. 8,735,553 to Li et al., incorporated by reference in its entirety.

In another embodiment, the antibody of the present disclosure comprises Fc domain of human IgG4 with S228P and/or R409K substitutions (according to EU numbering system).

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions or light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 5, SEQ ID NO: 9, SEQ ID NO: 7, SEQ ID NO: 12, SEQ ID NO: 14, SEQ ID NO: 16, SEQ ID NO: 18, SEQ ID NO: 42 or SEQ ID NO: 44.

The polynucleotides of the present disclosure can encode the variable region sequence of an anti-TAA (e.g., GPC3)×CD137 antibody. They can also encode both a variable region and a constant region of the antibody. Some of the polynucleotide sequences encode a polypeptide that comprises variable regions of both the heavy chain and the light chain of the exemplified anti-TAA (e.g., GPC3)×CD137 antibodies.

The disclosure further provides polynucleotides encoding the anti-GPC3×CD137 antibody described herein. In some aspects, the polynucleotide encoding the first polypeptide or the second polypeptide of anti-GPC3×CD137 antibody has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 22, SEQ ID NO: 26, SEQ ID NO: 28, SEQ ID NO: 30, SEQ ID NO: 32, SEQ ID NO: 34, or SEQ ID NO: 24. In some embodiments, the polynucleotides described herein could be codon-optimized for expression in host cells, e.g., eukaryotic cells, more specifically mammalian cells (e.g., CHO cells).

Also provided in the present disclosure are expression vectors and host cells for producing the antibodies herein, e.g., anti CD137 antibodies, and anti-TAA (e.g., GPC3)×CD137 antibodies. The choice of expression vector depends on the intended host cells in which the vector is to be expressed. Typically, the expression vectors contain a promoter and other regulatory sequences (e.g., enhancers) that are operably linked to the polynucleotides encoding an antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 1994; 20:125; and Bittner et al., Meth. Enzymol. 1987; 153:516, each incorporated by reference in their entirety). For example, the SV40 enhancer or CMV enhancer can be used to increase expression in mammalian host cells.

The host cells for harboring and expressing the antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express antibodies. Insect cells in combination with baculovirus vectors can also be used. In other aspects, mammalian host cells are used to express and produce the antibodies of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987, incorporated by reference in its entirety. Expression vectors for mammalian host cells can include expression control sequences, such as an origin of replication, a promoter, and an enhancer (see, e.g., Queen et al., Immunol. Rev. 1986; 89:49-68, incorporated by reference in its entirety), and necessary processing information sites, such as ribosome binding sites, RNA splice sites, polyadenylation sites, and transcriptional terminator sequences.

These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIll promoter, the constitutive MPSV promoter, the tetracycline-inducible CMV promoter (such as the human immediate-early CMV promoter), the constitutive CMV promoter, and promoter-enhancer combinations known in the art.

### Production of Bispecific Antibodies

The current standard for an engineered heterodimeric antibody Fc domain is the knobs-into-holes (KiH) design, which introduced mutations at the core CH3 domain interface. The resulted heterodimers have a reduced CH3 melting temperature (69° C. or less). On the contrary, the Zymeworks Azymetric™ Platform (supra) heterodimeric Fc design has a thermal stability of 81.5° C., which is comparable to the wild-type CH3 domain.

### Pharmaceutical Compositions

Also provided are compositions, including pharmaceutical formulations, comprising an antibody or antigen-binding fragment thereof herein, or polynucleotides comprising sequences encoding an antibody or antigen-binding fragment herein. In certain embodiments, compositions comprise one or more antibodies or antigen-binding fragments herein, or one or more polynucleotides comprising sequences encoding one or more antibodies or antigen-binding fragments herein. These compositions can further comprise suitable carriers, such as pharmaceutically acceptable excipients including buffers, which are well known in the art.

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

### Methods of Detection and Diagnosis

The antibodies or antigen-binding fragments of the present disclosure are useful in a variety of applications including, but not limited to, methods for the detection of CD137 or GPC3. In one aspect, the antibodies or antigen-binding fragments are useful for detecting the presence of CD137 or GPC3 in a biological sample. The term “detecting” as used herein includes quantitative or qualitative detection. In certain aspects, a biological sample comprises a cell or tissue. In other aspects, such tissues include normal and/or cancerous tissues that express CD137 or GPC3 at higher levels relative to other tissues.

In one aspect, the present disclosure provides a method of detecting the presence of CD137 or GPC3 in a biological sample. In certain aspects, the method comprises contacting the biological sample with the antibody herein under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of GPC3. In certain aspects, the method comprises contacting a test cell with an anti-GPC3×CD137 antibody; determining the level of expression (either quantitatively or qualitatively) of GPC3 expressed by the test cell by detecting binding of the anti-GPC3×CD137 antibody to the GPC3 polypeptide; and comparing the level of expression by the test cell with the level of GPC3 expression in a control cell (e.g., a normal cell of the same tissue origin as the test cell or a non-GPC3 expressing cell), wherein a higher level of GPC3 expression in the test cell as compared to the control cell indicates the presence of a disorder associated with expression of GPC3.

### VI. Methods of Treatment

Anti-CD137 antibody

The antibodies or antigen-binding fragments of the present disclosure are useful in a variety of applications including, but not limited to, methods for the treatment of a CD137-associated disorder or disease. In one aspect, the CD137-associated disorder or disease is a cancer. In the case of a CD137×TAA multispecific antibody, the cancer can be specific to the TAA, with CD137 acting to recruit immune cells to the TAA expressing tumor.

In one aspect, the present disclosure provides a method of treating cancer. In certain aspects, the method comprises administering to a patient in need thereof a therapeutically effective amount of an anti-CD137 antibody or antigen-binding fragment or CD137 containing multispecific antibody, or the pharmaceutical composition thereof. In another aspect, the present disclosure provides anti-CD137 antibody or antigen-binding fragment or the multispecific antibody, or the pharmaceutical composition for use in the treatment of cancer. In another aspect, the present disclosure provides the use of the anti-CD137 antibody or antigen-binding fragment, multispecific antibody or antigen-binding fragment thereof, or the pharmaceutical composition in the manufacture of a medicament for the treatment of cancer.

The cancer can include, without limitation, gastric cancer, colon cancer, pancreatic cancer, breast cancer, head and neck cancer, kidney cancer, liver cancer, lung cancer, small cell lung cancer, non-small cell lung cancer, ovarian cancer, skin cancer, mesothelioma, lymphoma, leukemia, myeloma and sarcoma.

### Anti-GPC3×CD137 Multispecific Antibody

The antibodies or antigen-binding fragments of the present disclosure are useful in a variety of applications including, but not limited to, methods for the treatment of a GPC3-associated disorder or disease. In one aspect, the GPC3-associated disorder or disease is a cancer.

In one aspect, the present disclosure provides a method of treating cancer. In certain aspects, the method comprises administering to a patient in need thereof a therapeutically effective amount of an anti-GPC3×CD137 antibody or antigen-binding fragment, or the pharmaceutical composition thereof. In another aspect, the present disclosure provides the multispecific antibody or antigen-binding fragment thereof, or the pharmaceutical composition for use in the treatment of cancer. In another aspect, the present disclosure provides the use of the multispecific antibody or antigen-binding fragment thereof, or the pharmaceutical composition in the manufacture of a medicament for the treatment of cancer.

In one embodiment, wherein the cancer expresses GPC3. In one embodiment, the cancer is an advanced or metastatic solid tumor.

The cancer can include, without limitation, any one or more of liver cancer, lung cancer, gastric cancer, germ cell tumors, thyroid cancer, pancreatic cancer, ovarian cancer, skin cancer, kidney cancer (e.g., Wilms tumor), esophageal carcinoma, atypical teratoid rhabdoid tumor of the brain, and undifferentiated synovial sarcoma. In one embodiment, the liver cancer is hepatoblastoma or hepatocellular carcinoma (HCC). In another embodiment, the lung cancer is non-small cell lung cancer (NSCLC) or small cell lung cancer (SCLC). In another embodiment, the non-small cell lung cancer is squamous non-small cell lung cancer. In another embodiment, the non-small cell lung cancer is GPC3+ squamous non-small cell lung cancer. In another embodiment, the gastric cancer is alpha-fetoprotein positive (AFP+) gastric cancer. In another embodiment, the kidney cancer is Wilms tumor. In another embodiment, the esophageal carcinoma is esophageal squamous cell carcinoma. In another embodiment, the esophageal carcinoma is GPC3+ esophageal squamous cell carcinoma. In another embodiment, the germ cell tumor is yolk sac tumors or non-dysgerminomas.

### Others

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician.

### VII. Combination Therapy

In one aspect, anti-CD137 antibodies or anti-CD137 containing multispecific antibodies of the present disclosure, e.g., anti-CD137×TAA antibodies or anti-GPC3×CD137 antibodies can be used in combination with other therapeutic agents.

The other therapeutic agents include, for example, other immune checkpoint antibodies. Such immune checkpoint antibodies can include anti-PD1 antibodies. Anti-PD1 antibodies can include, without limitation, Tislelizumab, Pembrolizumab or Nivolumab. Tislelizumab is disclosed in U.S. Pat. No. 8,735,553. Pembrolizumab (formerly MK-3475), is disclosed in U.S. Pat. Nos. 8,354,509 and 8,900,587 and is a humanized lgG4-K immunoglobulin which targets the PD1 receptor and inhibits binding of the PD1 receptor ligands PD-L1 and PD-L2. Nivolumab (as disclosed by Bristol-Meyers Squibb) is a fully human lgG4-K monoclonal antibody. Nivolumab (clone 5C4) is disclosed in U.S. Pat. No. 8,008,449 and WO 2006/121168.

Other immune checkpoint antibodies for combination with anti-CD137 antibodies or anti-CD137 containing multispecific antibodies of the present disclosure can include anti-TIGIT antibodies. Such anti-TIGIT antibodies can include without limitation, anti-TIGIT antibodies as disclosed in WO2019/129261.

In one embodiment, the present disclosure provides a use of the combination of anti-CD137 antibodies or anti-CD137 containing multispecific antibodies of the present disclosure, (e.g., anti-CD137×TAA antibodies or anti-GPC3×CD137 antibodies) and anti-PD-1 antibody (such as Tislelizumab or other anti-PD-1 antibody mentioned above) in the manufacture of a medicament for the treatment of cancer, such as the cancers mentioned above. In another embodiment, the present disclosure provides the combination of anti-CD137 antibodies or anti-CD137 containing multispecific antibodies of the present disclosure, (e.g., anti-CD137×TAA antibodies or anti-GPC3×CD137 antibodies) and anti-PD-1 antibody (such as Tislelizumab or other anti-PD-1 antibody mentioned above) for use in the treatment of cancer, such as the cancers mentioned above.

The combination therapy may refer to and include any one of the following:
simultaneous administration of such combination therapy to a patient in need of treatment, when such components are formulated together into a single dosage form which releases said components at substantially the same time to said patient,
substantially simultaneous administration of such combination to a patient in need of treatment, when such components are formulated apart from each other into separate dosage forms which are taken at substantially the same time by said patient, whereupon said components are released at substantially the same time to said patient,
sequential administration of such combination therapy to a patient in need of treatment, when such components are formulated apart from each other into separate dosage forms which are taken at consecutive times by said patient with a significant time interval between each administration, whereupon said components are released at substantially different times to said patient; and
sequential administration of such combination to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “CD137” or “TNFRSF9,” “ILA” or “41BB” or “4-1BB” refers to a co-stimulatory molecule belonging to the TNFRSF family. The nucleic acid sequence of human CD137 is set forth in SEQ ID NO: 36, based on GenBank sequence Accession No: NM_001561.4. The amino acid sequence of human CD137 is SEQ ID NO: 35.

The term “Glypican 3” (GPC3) is also known as DGSX, GTR2-2, MXR7, OCI-5, SDYS, SGB, SGBS, SGBS1. The amino acid sequence of human GPC3 (SEQ ID NO: 51) can also be found at NCBI Reference Sequence: NP_004475.1. The nucleic acid sequence of human GPC3 is set forth in SEQ ID NO: 52.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient comprising, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, chimeric antibodies, and anti-idiotypic (anti-Id) antibodies, a human engineered antibody, a single chain antibody (scFv), a single domain antibody, a Fab fragment, a Fab′ fragment, or a F(ab′)₂ fragment. The antibodies can be of any isotype/class (e.g., IgG, IgE, IgM, IgD, IgA and IgY), or subclass (e.g., IgG1, IgG2, IgG3, IgG4, IgA1 and IgA2). In addition, the antibody includes the derivative agents thereof, such as by linking to another agent (such as other drug) directly or indirectly or forming a complex with another agent. The term “antibody” herein comprises monospecific antibody, bispecific antibody and multispecific antibody.

The term “chimeric antibody” means molecules made up of domains from different species, i.e., fusing the variable domain of an antibody from one host species (e.g. mouse, rabbit, llama, etc.) with the constant domain of an antibody from a different species (e.g. human).

In some embodiments, the anti-GPC3 antibodies comprise at least one antigen-binding site, at least a variable region. In some embodiments, the anti-GPC3 antibodies comprise an antigen-binding fragment from an GPC3 antibody described herein. In some embodiments, the anti-GPC3 antibody is isolated or recombinant.

In some embodiments, the anti-CD137 antibodies comprise at least one antigen-binding site, at least a variable region. In some embodiments, the anti-CD137 antibodies comprise an antigen-binding fragment from an CD137 antibody described herein. In some embodiments, the anti-CD137 antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature. 1975; 256:495-497; U.S. Pat. No. 4,376,110; Ausubel et al., CURRENT PROTOCOLS IN MOLECULAR BIOLOGY, 1992; Harlow et al., ANTIBODIES: A LABORATORY MANUAL, Cold spring Harbor Laboratory, 1988; and Colligan et al., CURRENT PROTOCOLS IN IMMUNOLOGY, 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res. 2001; 29:205-206; Chothia and Lesk, J. Mol. Biol. 1987; 196:901-917; Chothia et al., Nature. 1989; 342:877-883; Chothia et al., J. Mol. Biol. 1992; 227:799-817; Al-Lazikani et al., J. Mol. Biol. 1997; 273:927-748; ImMunoGenTics (IMGT) numbering (Lefranc, M.-P., The Immunologist. 1999; 7, 132-136; Lefranc, M.-P. et al., Dev. Comp. Immunol. 27, 55-77 (2003) (“IMGT” numbering scheme)). Definitions of antigen combining sites are also described in the following: Ruiz et al., Nucleic Acids Res. 28:219-221 (2000); and Lefranc, M. P., Nucleic Acids Res., 29:207-209 (2001); MacCallum et al. J. Mol. Biol. 262:732-745 (1996); and Martin et al., Proc. Natl. Acad. Sci. USA. 86:9268-9272 (1989); Martin et al., Methods Enzymol. 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk, J. Mol. Biol. 1987; 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

“Antigen-binding domain” as used herein, means the portion on an antibody that specifically binds to an antigen. In some embodiments, it comprises at least six CDRs and specifically bind to an epitope (or three CDRs in terms of single domain antibody). An “antigen-binding domain” of a multispecific antibody (e.g., a bispecific antibody) comprises a first antigen binding domain that specifically binds to a first epitope and a second antigen binding domain specifically binds to a second epitope. Multispecific antibodies can be bispecific, trispecific, tetraspecific etc., with antigen binding domains directed to each specific epitope. Multispecific antibodies can be multivalent (e.g., a bispecific tetravalent antibody) that comprises multiple antigen binding domains, for example, 2, 3, 4 or more antigen binding domains that specifically bind to a first epitope and 2, 3, 4 or more antigen binding domains that specifically bind a second epitope.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine, rabbit, llama, etc.) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent/camelid antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

“2+2 format” means in a bispecific antibody targeting two different antigens or two different epitopes, such antibody comprises two first antigen binding domains that specifically bind to the first antigen or the first epitope, and two second antibody binding domains that specifically bind to the second antigen or the second epitope.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to GPC3 or to CD137. Specifically, common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces or VH/V_{L} interfaces of antibodies (see, e.g., US 2011/0287009, US2007/0178552, WO 96/027011, WO 98/050431, and Zhu et al., Protein Science. 1997; 6:781-788). In some embodiments, knob-into-holes insure the correct pairing of two different heavy chains together during the manufacture of multispecific antibodies. For example, multispecific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also insure correct pairing.

The term “knob” as used herein in the context of “knob-into-hole” technology refers to an amino acid change that introduces a protuberance (knob) into a polypeptide at an interface in which the polypeptide interacts with another polypeptide. In some embodiments, the other polypeptide has a hole mutation.

The term “hole” as used herein in the context of “knob-into-hole” refers to an amino acid change that introduces a socket or cavity (hole) into a polypeptide at an interface in which the polypeptide interacts with another polypeptide. In some embodiments, the other polypeptide has a knob mutation.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 1990; 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see, Henikoff and Henikoff, Proc. Natl. Acad. Sci. USA. 1989; 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 1988; 4: 11-17, which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an antibody herein is administered to the subject at the same time as, just before, or just after administration of an additional therapeutic agent. In certain embodiments, an antibody herein is administered as a co-formulation with an additional therapeutic agent.

### Equivalent

It is to be understood that while the present invention has been described in conjunction with the detailed description thereof, the foregoing description is intended to illustrate and not limit the scope of the invention, which is defined by the scope of the appended claims. Other aspects, advantages, and modifications are within the scope of the following claims.

It is to be understood that one, some, any or all of the features of the various embodiments described herein may be combined to form further embodiments of the present disclosure. These and other aspects of the present disclosure will become apparent to those skilled in the art.

### EXAMPLES

### Example 1. Generation of Single Chain Anti-huCD137 VHH Antibody

CD137 recombinant proteins for phage campaign and binding assays

To discover VHH antibodies against human CD137, several recombinant proteins were designed and expressed for phage panning and screening. The cDNA coding regions for the full-length human CD137 (huCD137) was ordered based on the CD137 GenBank sequence (Accession No: NM_001561.4, the gene is available from Sinobio, Cat.: HG10041-M and is referenced herein as SEQ ID NO: 36). Human CD137 ligand (TNFSF9) was ordered based on the CD137 ligand GenBank sequence (Accession No: NM_003811.3, the gene is available from Sinobio, Cat.: HG15693-G). In brief, the coding region of extracellular domain (ECD) consisting of amino acid (AA) 24-183 of huCD137 (SEQ ID NO: 35), and the coding region of ECD consisting of AA 71-254 of huCD137 ligand (SEQ ID NO: 37) were PCR-amplified, respectively. The coding region of mIgG2a Fc (SEQ ID NO: 39) was PCR-amplified, and then conjugated with ECDs of human CD137 or ECD of human CD137 ligand by overlap-PCR to make mIgG2a Fc-fusion proteins. PCR products were then cloned into a pcDNA3.1-based expression vector (Invitrogen, Carlsbad, CA, USA), which resulted in two recombinant mIgG2a Fc-fusion protein expression plasmids, human CD137 ECD-mIgG2a, human CD137 ligand ECD-mIgG2a. Alternatively the coding regions of ECD consisting of AA 24-183 of huCD137 (SEQ ID NO: 35) were also cloned into a pcDNA3.1-based expression vector (Invitrogen, Carlsbad, CA, USA) with C-terminus fused with 6×His tags, which resulted in human CD137-ECD-his. For the recombinant fusion protein production, plasmids were transiently transfected into a HEK293-based mammalian cell expression system (developed in house) and cultured for 5-7 days in a CO₂ incubator equipped with rotating shaker. The supernatants containing the recombinant proteins were collected and cleared by centrifugation. Recombinant proteins were purified by Protein A column (Cat.: 17127901, GE Life Sciences) or Ni-NTA agarose (Cat.: R90115, Invitrogen). All recombinant proteins were dialyzed against phosphate buffered saline (PBS) and stored in −80° C. freezer in small aliquots.

### Llama Immunization and Phage Library Construction

One llama was immunized with human CD137 ECD-mIgG2a. Two weeks after the fourth immunization, llama PBMC were collected for RNA extraction, using the standard techniques (Chomczynski, et al., Single-step method of RNA isolation by acid guanidinium thiocyanate-phenol-chloroform extraction, Analytical. Biochem. 1987; 162(1):156-159).

Phage library was constructed by reverse-transcription and splice-overlap extension PCR. The PCR products were double-digested by NcoI/NotI and ligated into the phagemid vector pCANTAB-5E. Repertoires were then transformed into Escherichia coli TG1 bacteria and validated by DNA Sanger sequencing of random clones (>96 clones analyzed). Phages were purified by two precipitations with PEG/NaCl directly from the culture supernatant after a rescue step using KM13 helper phage. A library with size >10⁷ was obtained after transformation into E. coli bacteria.

### Phage Display Panning and Screening

Phage display selection was carried out by phage display using standard protocols (Silacci et al., (2005) Proteomics, 5, 2340-50; Zhao et al., (2014) PLoS One, 9, e111339). In brief, 10 μg/ml of immobilized human CD137 ECD-mIgG2a in immunotubes (Cat. 470319, ThermoFisher) was utilized in round 1 and 2. Hut78/huCD137 cells were used for selection in round 3. Immunotubes were blocked with 5% milk powder (w/v) in PBS supplemented with 1% Tween 20 (MPBST) for 1 hour. After washes with PBST (PBS buffer supplemented with 0.05% Tween 20), 5×10¹² (round 1) or 5×10¹¹ (rounds 2) phages from each sub library were depleted by human CD40 ECD-mIgG2a in MPBST for 1 hour and then incubated with the antigen for 1 hour. For the third rounds of selections, cell panning was carried out using Hut78/huCD137 cells with HEK293 (ATCC, CRL-1573) cells as depletion cells. After washes with PBST, bound phages were eluted with 100 mM triethylamine (Sigma-Aldrich). Eluted phages were used to infect mid-log phase E. coli TG1 bacteria and plated onto TYE-agar plates supplemented with 2% glucose and 100 μg/ml ampicillin. After three rounds of selections, individual clones were picked up and phage containing supernatants were prepared using standard protocols. Phage ELISA were used to screen anti-huCD137 VHH antibodies.

For phage ELISA, a Maxisorp immunoplate was coated with antigens and blocked with 5% milk powder (w/v) in PBS buffer. Phage supernatant was blocked with MPBST for 30 minutes and added to wells of the ELISA plate for 1 hour. After washes with PBST, bound phage was detected using HRP-conjugated anti-M13 antibody (GE Healthcare) and 3,3′,5,5′-tetramethylbenzidine substrate (Cat.: 00-4201-56, eBioscience, USA). CD137-expressing cells (10⁵ cells/well) were incubated with ELISA-positive phage supernatants, followed by binding with Alexa Fluro-647-labeled anti-M13 antibody (GE Healthcare). Cell fluorescence was quantified using a flow cytometer (Guava easyCyte™ 8HT, Merck-Millipore, USA).

### Expression and Purification of Fe Fusion VHH Antibodies

Anti-huCD137 VHH antibodies were then constructed as human Fc fusion VHH antibody format (VHH-Fc) using in-house developed expression vectors. VHH domain antibodies were fused at the N terminal of human Fc with a G4S linker (SEQ ID NO: 62) in between. A Fc-null version (an inert Fc without FcγR-binding, SEQ ID NO: 19) of human IgG1 was used. Expression and preparation of Fc fusion VHH antibodies were achieved by transfection into 293G cells and by purification using a Protein A column (Cat. No. 17543802, GE Life Sciences). The purified antibodies were concentrated to 0.5-5 mg/mL in PBS and stored in aliquots in a −80° C. freezer.

### Example 2. Characterization of Purified Anti-huCD137 VHH Antibodies

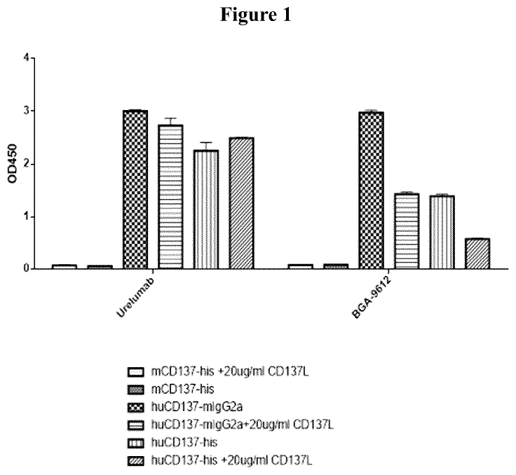

For antigen ELISA, a Maxisorp immunoplate was coated with antigens and blocked with 3% BSA (w/v) in PBS buffer (blocking buffer). Monoclonal VHH antibodies were blocked with blocking buffer for 30 minutes and added to wells of the ELISA plate for 1 hour. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA). Ligand competition was also applied in a ELISA assay. The ELISA analysis and the ligand competition result of one representative clone BGA-9612 are shown in FIG. 1. The result showed that BGA-9612 (SEQ ID NOs: 1-5) could bind to human CD137 with good affinity, but showed no binding to mouse CD137. The binding to human CD137 with BGA-9612 could be reduced by competition with huCD137 ligand.

### Example 3. Humanization of the Anti-Human CD137 VHH BGA-9612

For humanization of the BGA-9612, human germline IgG genes were searched for sequences that share high degrees of homology to the cDNA sequences of BGA-9612 variable regions by blasting the human immunoglobulin gene database in IMGT and NCBI websites. The human IGVH genes that are present in human antibody repertoires with high frequencies (Glanville 2009 PNAS 106:20216-20221) and are highly homologous to BGA-9612 were selected as the templates for humanization.

Humanization was carried out by CDR-grafting (Methods in Molecular Biology, Vol 248: Antibody Engineering, Methods and Protocols, Humana Press) and the humanized VHHs from BGA-9612 were engineered as Fc-VHH format using an in-house developed expression vector. Humanized VHHs from BGA-9612 were fused to the C-terminal of Fc with a G4S linker (SEQ ID NO: 62) as Fc-VHH format using in-house developed expression vectors that contain Fc variant of a human IgG1 (SEQ ID NO: 19), with easy adapting sub-cloning sites. Expression and preparation of humanized VHHs from BGA-9612 was achieved by transfection of the constructs into ExpiCHO™ cells and by purification using a Protein A column. The purified antibodies were concentrated to 0.5-5 mg/mL in PBS and stored in aliquots in −80° C. freezer.

### Framework Swapping

In the initial round of humanization, mutations from camelid to human amino acid residues in framework regions were guided by the simulated 3D structure, and the camelid framework residues having structural importance for maintaining the canonical structures of CDRs were retained in the 1^{st} round of humanization, including amino acid residues R27, F37, E44, R45, Q46, Y47, G49, V78, 194, and Q103 (Kabat Numbering). Specifically, CDRs of BGA-9612 VHH (SEQ ID NOs: 1-3) were grafted into the framework of human germline variable gene IGVH3-23 with several camelid framework residues retained and resulted in BGA-6582 (SEQ ID NO: 8-9). The binding affinities of BGA-9612 and BGA-6582 by SPR are shown in Table 2.

In the examples, Kabat numbering and Kabat definition were used for sequences of CDRs and VH/VL. EU numbering was used for Fc sequences.

TABLE 2
Binding affinities of BGA-9612 and BGA-6582 by SPR

| VHH | kₒₙ (M⁻¹s⁻¹) | k_{off} (s⁻¹) | K_{D} (M) |
|---|---|---|---|
| BGA-9612 | 2.07E+04 | 4.19E−04 | 2.03E−08 |
| BGA-6582 | 2.12E+04 | 5.88E−04 | 2.77E−08 |

Based on BGA-6582, we made several single-mutations converting the retained camelid residues in framework region to corresponding human germline residues and the combination of single-mutations, as shown in Table 3. All humanization mutations were made using primers containing mutations at specific positions and a site directed mutagenesis kit (Cat. No. FM111-02, TransGen, Beijing, China). The desired mutations were verified by sequencing analysis. These further humanized VHH from BGA-6582 were tested in SPR binding assays, as shown in Table 3. BGA-3726 (SEQ ID NOs: 1-3 and 6-7) including amino acid Q46E based on BGA-6582 was selected for further engineering. Amino acid residues F37, Y47, G49, and 194 in the framework region are critical for binding to CD137 (Kabat numbering).

TABLE 3
Binding affinities of variants having single amino acid mutation based on BGA-6582

| | Mutation on | | | | SEC-HPLC |
|---|---|---|---|---|---|
| VHH | BGA 6582 | kₒₙ (M⁻¹s⁻¹) | k_{off} (s⁻¹) | K_{D} (M) | purity |
| BGA-6582 | | 4.35E+04 | 5.93E−04 | 1.36E−08 | 93.74% |
| BGA-6582-F37V | F37V | No binding | No binding | No binding | — |
| BGA-6582-E44G | E44G | 2.64E+04 | 2.42E−04 | 9.16E−09 | 92.96% |
| BGA-6582-R45L | R45L | 2.78E+04 | 4.13E−04 | 1.48E−08 | 94.52% |
| BGA-6582-Q46E | Q46E | 3.02E+04 | 4.32E−04 | 1.43E−08 | 97.85% |
| (BGA-3726) | | | | | |
| BGA-6582-Y47W | Y47W | No binding | No binding | No binding | — |
| BGA-6582-G49S | G49S | No binding | No binding | No binding | — |
| BGA-6582-V78L | V78L | 4.35E+04 | 5.93E−04 | 1.36E−08 | 57.22% |
| BGA-6582-I94K | I94K | No binding | No binding | No binding | — |
| BGA-6582-Q103W | Q103W | 2.73E+04 | 2.44E−04 | 8.96E−09 | 98.38% |

### Biophysical Property Improvement

Humanized VIUH BGA-3726 did not show superior overall biophysical property (e.g., Tm or Tagg) as camelid VIUH BGA-9612 (data not shown). Thus, BGA-3726 was further engineered by introducing mutations in CDRs and framework regions to improve biophysical properties for therapeutic use in human.

Amino acid N73 at framework region 3 (FR3) of BGA-3726 was identified as a hot spot for deamination. To mitigate the post-translational modification (PTM) risk, the subsequent amino acid S74 was mutated to alanine (back mutation to camelid amino acid residue).

Taken together, based on BGA-3726, the following engineered versions of humanized VHHs were derived from the mutation process described as above: (1) BGA-3544 containing amino acid N64K and N65G in HCDR2 and L5V, S82bN, A84P and L108Q in framework (Kabat numbering) (SEQ ID NOs: 1, 10, 3, and 11-12), (2) BGA-7031 containing L5V, S82bN, A84P and L108Q in framework (Kabat numbering) (SEQ ID NOs: 1-3, and 15-16), (3) BGA-9502 containing amino acid N64K and N65G in HCDR2 and L5V, S74A, S82bN, A84P and L108Q in framework (Kabat numbering) (SEQ ID NOs: 13-14), (4) BGA-2524 containing L5V, S74A, S82bN, A84P and L108Q in framework (Kabat numbering) (SEQ ID NOs: 17-18).

For affinity determination, antibodies were captured by anti-human Fe surface, and used in the affinity—assay based on surface plasmon resonance (SPR) technology. The results of SPR-determined binding profiles of anti-CD137 antibodies were summarized in Table 4. BGA-3544 and BGA-7031 have similar binding affinities with dissociation constant at 17.6 nM and 11.7 nM, respectively, which are comparable to that of BGA-9612 (15.2 nM). BGA-6582 also has comparable binding affinity with BGA-9612.

TABLE 4
Comparison on binding affinities of BGA-9612 and humanized VHHs by SPR

| | Test 1 | | | Test 2 | | |
|---|---|---|---|---|---|---|
| VHH | kₒₙ (M⁻¹s⁻¹) | k_{off} (s⁻¹) | K_{D} (nM) | kₒₙ (M⁻¹s⁻¹) | k_{off} (s⁻¹) | K_{D} (M) |
| BGA- | 2.07E+04 | 4.19E−04 | 2.03E−08 | 4.95E+04 | 7.54E−04 | 1.52E−08 |
| 9612 | | | | | | |
| BGA- | 2.12E+04 | 5.88E−04 | 2.77E−08 | — | — | — |
| 6582 | | | | | | |
| BGA- | — | — | — | 5.69E+04 | 9.99E−04 | 1.76E−08 |
| 3544 | | | | | | |
| BGA- | — | — | — | 6.16E+04 | 7.19E−04 | 1.17E−08 |
| 7031 | | | | | | |

The biophysical properties of chimeric and humanized Fc-VHHs were tested. The tested biophysical properties included melting temperature by DSC, aggregation temperature by SLS266, hydrophobicity by HIC-HPLC and self-association tendency by AC-SINS (see below the detailed description). BGA-9612 showed optimal thermal stability by Tm and Tagg and good colloidal stability by AC-SINS. As shown in Table 5, humanized VHHs BGA-3544 and BGA-7031 showed comparable overall biophysical property as BGA-9612 (chimeric). In addition, BGA-3544 and BGA-7031 showed improved Tm compared with BGA-9612 (chimeric).

TABLE 5
Summary of biophysical properties of anti-CD137 VHHs

| | Tm by | Tagg by | HIC | |
|---|---|---|---|---|
| | DSC | SLS266 | Retention | AC-SINS |
| Fc-VHH | (° C.) | (° C.) | Time (min) | Δλ (nm) |
| BGA-9612 | 66.84 | 72.50 | 22.27 | −0.2 |
| BGA-3544 | 70.28 | 71.35 | 21.55 | 0.9 |
| BGA-7031 | 68.29 | 72.63 | 22.72 | 0.1 |

Melting temperature (Tm) was determined using a high throughput MicroCal™ VP-Capillary DSC (Malvern Instruments, Northampton, MA). Thermograms for each protein (350 L at 0.5 mg/mL) were obtained from 20° C. to 100° C. using a scan rate of 60° C./hr. Thermograms of the buffer alone were subtracted from each protein sample. Obtained results will show the values for midpoint of transition temperatures (Tm) and the calorimetric enthalpy (ΔH) of the sample.

The aggregation temperature Tagg (° C.) is representative of the colloidal stability of the samples and was obtained by monitoring the onset of aggregation by SLS266 using UNCLE™ (Unchained lab, Pleasanton, CA). Samples were loaded into Uni, and subjected to a temperature ramping from 15° C. to 95° C. The back-reflection optics cannot detect near UV light scattering by protein aggregates, and thus only non-scattered light reaches the detector. The reduction of back reflected light is therefore a direct measure for aggregation in the sample.

To determine the hydrophobicity of a given VHH using HPLC e2695 (Waters Corporation, Milford, MA), the protein sample was diluted in mobile phase A (50 mM sodium phosphate, 1.5 M ammonium sulfate, pH 7.0) and was subsequently filtered prior to loading on a MAbPac™ HIC-10 column (Thermo Fisher Scientific, Waltham, MA) equilibrated in mobile phase A. The samples were eluted using an inverted gradient from mobile phase A to mobile phase B (50 mM sodium phosphate pH 7.0). The elution was followed by recording the A280 nm fraction as a function of time, and the data was then exported and analyzed using the Empower™ software. The retention time of each sample was compared to a reference and is characteristic of the VHH hydrophobicity with longer elution times correlating with higher degree of hydrophobicity.

The AC-SINS assay measures protein self-interaction by capturing the VHH on the surface of a gold colloid that displays surface resonance oscillations in frequency with visible light. As the immobilized antibodies self-interact, the colloids aggregate, changing the oscillation frequency to absorb at a longer wavelength. Gold nanoparticles were incubated with an 80/20 (v/v) capture antibody/non-capture antibody mixture. The coated gold nanoparticles were then concentrated 10× into PBS. The samples were pre-diluted in PBS at concentration of 50 g/ml. 10 μL of the 10× concentrated AuNPs were incubated with 100 μL of samples in a 384-well plate in dark at room temperature for 2 hours. Then the absorbance spectra of each well from 510 to 570 nm was read using BMG ClarioStar™ (BMG Labtech, Offenburg, Germany). The red-shifting of the maximum of absorption peak and its strength is indicative of the self-interaction propensity of the tested VHH samples.

### Example 4. Binding Activities of Humanized VHHs to Native CD137

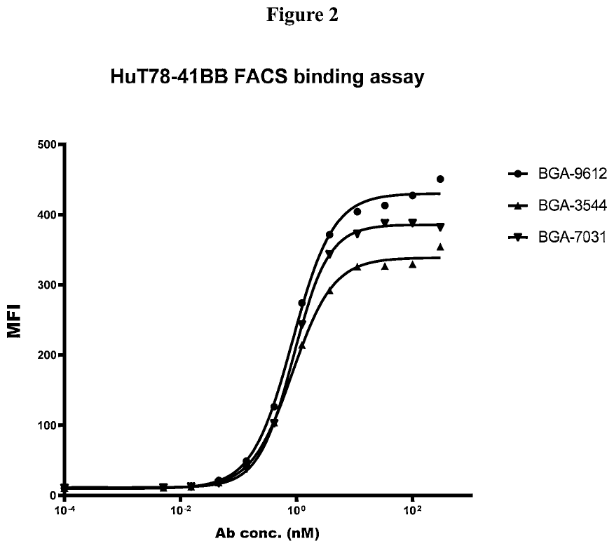

To evaluate the binding activity of anti-CD137 antibody to bind native CD137 on live cells, HuT78 cells were engineered to over-express human CD137. Live HuT78/CD137 cells were seeded in a 96-well plate, and were incubated with a series of dilutions of anti-CD137 antibody. Goat anti-Human IgG was used as secondary antibody to detect antibody binding to the cell surface. EC₅₀ values for dose-dependent binding to human native CD137 were determined by fitting the dose-response data to the four-parameter logistic model with GraphPad Prism™. The data is shown in FIG. 2 and Table 6. The humanized VHHs retained sub-nanomolar binding affinity to native CD137.

TABLE 6
Binding of chimeric and humanized VHHs to HuT78/CD137

| VHH | HuT78/CD137 binding EC50(nM) |
|---|---|
| BGA-9612 | 0.85 |
| BGA-3544 | 0.85 |
| BGA-7031 | 0.88 |

### Example 5. Anti-GPC3×CD137 Multispecific Antibodies

Agonistic anti-huCD137 antibodies have demonstrated toxicity in the clinical setting, which may indicate that systemic FcγR cross-linking is not ideal for CD137 activation. The aim was to achieve potent CD137 stimulation specifically at the tumor site without systemic CD137 activation for a broad range of cancers. To overcome the dependency of FcγR cross-linking, we generated an anti-GPC3×CD137 multispecific antibody with the following features as shown in FIG. 3. This specific construct included an IgG-fusion like multispecific antibody format with a module ratio of 2:2, a bivalent F(ab′)2 fragment that binds to human GPC3, VH domain fragments with a fusion at the C terminal of CH3, which bind huCD137, and a Fc null version of huIgG1 (IgG1mf Fc, SEQ ID NO: 53), which has no FcγR binding but retains FcRn binding. The amino acid and DNA sequences of GPC3 antibody are shown in Table 7.

TABLE 7
Amino acid and DNA sequences of GPC3 antibody

| | | |
|---|---|---|
| GPC3 | SEQ ID | QVQLVQSGAEVKKPGASVKVSCKASGYTFTDYEMHWVRQAPGQGLEWM |
| VH AA | NO: 41 | GALDPKTGDTAYSQKFKGRVTLTADKSTSTAYMELSSLTSEDTAVYYCTRFY |
| | | SYTYWGQGTLVTVSS |
| GPC3 | SEQ ID | CAGGTGCAGCTGGTGCAGTCTGGAGCAGAGGTGAAGAAGCCAGGAGCC |
| VH | NO: 42 | AGCGTGAAGGTGTCCTGCAAGGCCTCTGGCTATACCTTCACAGACTACG |
| DNA | | AGATGCACTGGGTGCGGCAGGCACCAGGACAGGGACTGGAGTGGATGG |
| | | GCGCCCTGGACCCTAAGACCGGCGATACAGCCTATAGCCAGAAGTTTAA |
| | | GGGCAGAGTGACCCTGACAGCCGACAAGTCCACCTCTACAGCCTACATG |
| | | GAGCTGAGCTCCCTGACCAGCGAGGATACAGCCGTGTACTATTGTACCC |
| | | GGTTCTACTCCTATACATACTGGGGCCAGGGCACCCTGGTGACAGTGTCT |
| | | AGC |
| GPC3 | SEQ ID | DVVMTQSPLSLPVTPGEPASISCRSSQSLVHSNRNTYLHWYLQKPGQSPQLL |
| VL | NO: 43 | IYKVSNRFSGVPDRFSGSGSGTDFTLKISRVEAEDVGVYYCSQNTHVPPTFG |
| AA | | QGTKLEIK |
| GPC3 | SEQ ID | GACGTGGTCATGACACAGTCCCCTCTGTCTCTGCCAGTGACCCCAGGAG |
| VL | NO: 44 | AGCCTGCCTCTATCAGCTGCAGGAGCTCCCAGAGCCTGGTGCACTCCAA |
| DNA | | CCGCAATACATACCTGCACTGGTATCTGCAGAAGCCAGGCCAGTCCCCCC |
| | | AGCTGCTGATCTACAAGGTGTCTAACCGGTTCAGCGGCGTGCCCGACAG |
| | | ATTTTCCGGCTCTGGCAGCGGCACCGATTTCACACTGAAGATCTCCCGGG |
| | | TGGAGGCAGAGGATGTGGGCGTGTACTATTGTTCTCAGAATACCCACGT |
| | | GCCCCCTACATTTGGCCAGGGCACCAAGCTGGAGATCAAG |
| Anti-GPC3 | SEQ ID | DYEMH |
| antibody | NO: 45 | |
| HCDR1 | | |
| (Kabat) | | |
| Anti-GPC3 | SEQ ID | ALDPKTGDTAYSQKFKG |
| antibody | NO: 46 | |
| HCDR2 | | |
| (Kabat) | | |
| Anti-GPC3 | SEQ ID | FYSYTY |
| antibody | NO: 47 | |
| HCDR3 | | |
| (Kabat) | | |
| Anti-GPC3 | SEQ ID | RSSQSLVHSNRNTYLH |
| antibody | NO: 48 | |
| LCDR1 | | |
| (Kabat) | | |
| Anti-GPC3 | SEQ ID | KVSNRFS |
| antibody | NO: 49 | |
| LCDR2 | | |
| (Kabat) | | |
| Anti-GPC3 | SEQ ID | SQNTHVPPT |
| antibody | NO: 50 | |
| LCDR3 | | |
| (Kabat) | | |
| Human | SEQ ID | MAGTVRTACLVVAMLLSLDFPGQAQPPPPPPDATCHQVRSFFQRLQPGLKW |
| GPC3 full | NO: 51 | VPETPVPGSDLQVCLPKGPTCCSRKMEEKYQLTARLNMEQLLQSASMELKF |
| length | | LIIQNAAVFQEAFEIVVRHAKNYTNAMFKNNYPSLTPQAFEFVGEFFTDVSL |
| AA | | YILGSDINVDDMVNELFDSLFPVIYTQLMNPGLPDSALDINECLRGARRDLK |
| | | VFGNFPKLIMTQVSKSLQVTRIFLQALNLGIEVINTTDHLKFSKDCGRMLTR |
| | | MWYCSYCQGLMMVKPCGGYCNVVMQGCMAGVVEIDKYWREYILSLEEL |
| | | VNGMYRIYDMENVLLGLFSTIHDSIQYVQKNAGKLTTTIGKLCAHSQQRQ |
| | | YRSAYYPEDLFIDKKVLKVAHVEHEETLSSRRRELIQKLKSFISFYSALPGYI |
| | | CSHSPVAENDTLCWNGQELVERYSQKAARNGMKNQFNLHELKMKGPEPV |
| | | VSQIIDKLKHINQLLRTMSMPKGRVLDKNLDEEGFESGDCGDDEDECIGGS |
| | | GDGMIKVKNQLRFLAELAYDLDVDDAPGNSQQATPKDNEISTFHNLGNVH |
| | | SPLKLLTSMAISVVCFFFLVH |
| Human | SEQ ID | ATGGCCGGGACCGTGCGCACCGCGTGCTTGGTGGTGGCGATGCTGCTCA |
| GPC3 full | NO: 52 | GCTTGGACTTCCCGGGACAGGCGCAGCCCCCGCCGCCGCCGCCGGACG |
| length | | CCACCTGTCACCAAGTCCGCTCCTTCTTCCAGAGACTGCAGCCCGGACT |
| DNA | | CAAGTGGGTGCCAGAAACTCCCGTGCCAGGATCAGATTTGCAAGTATGT |
| | | CTCCCTAAGGGCCCAACATGCTGCTCAAGAAAGATGGAAGAAAAATACC |
| | | AACTAACAGCACGATTGAACATGGAACAGCTGCTTCAGTCTGCAAGTAT |
| | | GGAGCTCAAGTTCTTAATTATTCAGAATGCTGCGGTTTTCCAAGAGGCCT |
| | | TTGAAATTGTTGTTCGCCATGCCAAGAACTACACCAATGCCATGTTCAAG |
| | | AACAACTACCCAAGCCTGACTCCACAAGCTTTTGAGTTTGTGGGTGAAT |
| | | TTTTCACAGATGTGTCTCTCTACATCTTGGGTTCTGACATCAATGTAGATG |
| | | ACATGGTCAATGAATTGTTTGACAGCCTGTTTCCAGTCATCTATACCCAG |
| | | CTAATGAACCCAGGCCTGCCTGATTCAGCCTTGGACATCAATGAGTGCCT |
| | | CCGAGGAGCAAGACGTGACCTGAAAGTATTTGGGAATTTCCCCAAGCTT |
| | | ATTATGACCCAGGTTTCCAAGTCACTGCAAGTCACTAGGATCTTCCTTCA |
| | | GGCTCTGAATCTTGGAATTGAAGTGATCAACACAACTGATCACCTGAAG |
| | | TTCAGTAAGGACTGTGGCCGAATGCTCACCAGAATGTGGTACTGCTCTTA |
| | | CTGCCAGGGACTGATGATGGTTAAACCCTGTGGCGGTTACTGCAATGTG |
| | | GTCATGCAAGGCTGTATGGCAGGTGTGGTGGAGATTGACAAGTACTGGA |
| | | GAGAATACATTCTGTCCCTTGAAGAACTTGTGAATGGCATGTACAGAATC |
| | | TATGACATGGAGAACGTACTGCTTGGTCTCTTTTCAACAATCCATGATTCT |
| | | ATCCAGTATGTCCAGAAGAATGCAGGAAAGCTGACCACCACTATTGGCA |
| | | AGTTATGTGCCCATTCTCAACAACGCCAATATAGATCTGCTTATTATCCTG |
| | | AAGATCTCTTTATTGACAAGAAAGTATTAAAAGTTGCTCATGTAGAACAT |
| | | GAAGAAACCTTATCCAGCCGAAGAAGGGAACTAATTCAGAAGTTGAAGT |
| | | CTTTCATCAGCTTCTATAGTGCTTTGCCTGGCTACATCTGCAGCCATAGCC |
| | | CTGTGGCGGAAAACGACACCCTTTGCTGGAATGGACAAGAACTCGTGG |
| | | AGAGATACAGCCAAAAGGCAGCAAGGAATGGAATGAAAAACCAGTTCA |
| | | ATCTCCATGAGCTGAAAATGAAGGGCCCTGAGCCAGTGGTCAGTCAAAT |
| | | TATTGACAAACTGAAGCACATTAACCAGCTCCTGAGAACCATGTCTATGC |
| | | CCAAAGGTAGAGTTCTGGATAAAAACCTGGATGAGGAAGGGTTTGAAA |
| | | GTGGAGACTGCGGTGATGATGAAGATGAGTGCATTGGAGGCTCTGGTGA |
| | | TGGAATGATAAAAGTGAAGAATCAGCTCCGCTTCCTTGCAGAACTGGCC |
| | | TATGATCTGGATGTGGATGATGCGCCTGGAAACAGTCAGCAGGCAACTC |
| | | CGAAGGACAACGAGATAAGCACCTTTCACAACCTCGGGAACGTTCATTC |
| | | CCCGCTGAAGCTTCTCACCAGCATGGCCATCTCGGTGGTGTGCTTCTTCT |
| | | TCCTGGTGCACTGA |

To further increase the in vivo half-life of GPC3×CD137 multispecific antibodies, YTE mutation (M252Y/S254T/T256E, EU numbering) located at CH2 of IgG Fe region were introduced in IgG1mf Fc to generate IgG1mf Fc-YTE (SEQ ID NO: 20).

The following GPC3×CD137 multispecific antibodies were generated.

BE-933: GPC3 antibody (SEQ ID Nos: 41-50) was combined with CD137 VHH BGA-9612 (SEQ ID NOs: 1-5), resulting in construct BE-933 (SEQ ID NOs: 23-24 for VL, SEQ ID NOs: 27-28 for VH).

BE-774: YTE mutation in Fc were introduced in the BE-933 to produce BE-774 (SEQ ID NOs: 23-24 for VL, SEQ ID NOs: 25-26 for VH).

BE-653: GPC3 antibody (SEQ ID Nos: 41-50) was combined with CD137 VHH BGA-7031 (SEQ ID Nos: 1-3 and 15-16), resulting in construct BE-653 (SEQ ID NOs: 23-24 for VL, SEQ ID NOs: 29-30 for VH).

BE-915: GPC3 antibody (SEQ ID Nos: 41-50) was combined with CD137 VHH BGA-2524 (SEQ ID Nos: 1-3 and 17-18) as well as YTE mutation in Fe, resulting in construct BE-915 (SEQ ID NOs: 23-24 for VL, SEQ ID NOs: 21-22 for VH).

BE-647: GPC3 antibody (SEQ ID Nos: 41-50) was combined with CD137 VHH BGA-9502 (SEQ ID Nos: 1, 10, 3, 13-14), resulting in construct BE-647 (SEQ ID NOs: 23-24 for VL, SEQ ID NOs: 31-32 for VH).

BE-621: GPC3 antibody (SEQ ID Nos: 41-50) was combined with CD137 VHH BGA-9502 (SEQ ID Nos: 1, 10, 3, 13-14) as well as YTE mutation in Fc, resulting in construct BE-621 (SEQ ID NOs: 23-24 for VL, SEQ ID NOs: 33-34 for VH).

### Example 6. Target Binding Activities of Anti-GPC3×CD137 Bispecific Antibodies

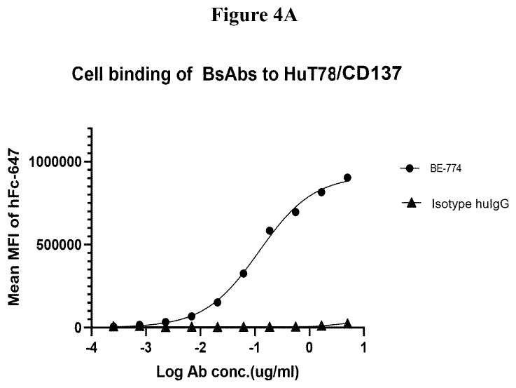

BE-774 was characterized for its binding kinetics by SPR assays using BIAcore™ T-200 (GE Life Sciences). Briefly, anti-kappa antibody was immobilized on an activated CM5 biosensor chip (Cat.: BR100839, GE Life Sciences). The BE-774 was flowed through the chip surface and captured by anti-kappa antibody. Then a serial dilution (6.0 nM to 2150 nM) of human CD137 ECD-mIgG2a or huGPC3-His were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The result demonstrated that BE-774 showed binding to huCD137 and huGPC3, as shown in Table 8 below. To evaluate the binding activity of the BE-774 bispecific antibody to native huCD137 on living cells, Hut78 cells were transfected to over-express human CD137. Live Hut78/huCD137 expressing cells were seeded in 96-well plates and were incubated with a serial dilution of BE-774. Goat anti-Human IgG was used as secondary antibody to detect antibody binding to the cell surface. EC₅₀ values for dose-dependent binding to human native CD137 were determined by fitting the dose-response data to the four-parameter logistic model with GraphPad Prism™. As shown in FIG. 4A, BE-774 demonstrated specific binding to native CD137 on living cells in a dose-responsive manner with EC50 of 0.7 nM. To evaluate the binding to huGPC3 on living cells, HepG2 cells that express huGPC3 were seeded in 96-well plates and were incubated with a serial dilution of BE-774. Goat anti-Human IgG was used as secondary antibody to detect antibody binding to the cell surface. EC₅₀ values for dose-dependent binding to human native GPC3 were determined by fitting the dose-response data to the four-parameter logistic model with GraphPad Prism™. As shown in FIG. 4B, BE-774 demonstrated specific binding to native GPC3 on living cells in a dose-responsive manner with an EC50 of 0.9 nM.

TABLE 8
SPR affinity of BE-774 to huGPC3 and huCD137

| | Bispecific | SPR KD (M) | | | |
|---|---|---|---|---|---|
| Antigen | Name | ka (1/Ms) | kd (1/s) | KD (M) | Rmax (Ru) |
| huGPC3 | BE-774 | 2.18E+05 | 2.20E−04 | 1.01E−09 | 170.7 |
| huCD137 | | 8.04E+05 | 4.25E−04 | 5.29E−10 | 140 |

### Example 7. Anti-GPC3×CD137 Induces T Cell Activation Cocultured with GPC3 Positive Tumor Cells

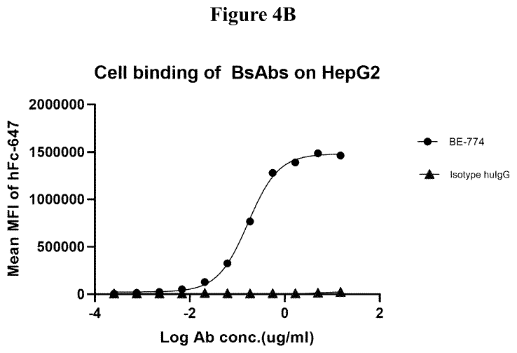

The functional activity of anti-GPC3×CD137 bispecific antibody was assessed in in vitro co-culture experiments using human peripheral blood mononuclear cell (PBMC) and OS8-expressing hepatocellular carcinoma (HCC) cell lines. OS8 is a single chain variable fragment (scFv) of an anti-human CD3 antibody OKT3 fused to the C-terminal domain (113-220 aa) of mouse CD8a which includes hinge, transmembrane and cytoplasmic domains. When expressed on target cells, OS8 could provide signal 1 for T cell activation (FIG. 5A). GPC3 high expressing HepG2 cells were chosen to evaluate the functional activity of GPC3×CD137 bispecific antibody, while SK-HEP-1 (GPC3 negative) were used as negative control cell lines.

Frozen human PBMC (AllCells) were thawed in RPMI 1640 medium and incubated at 37° C. overnight. OS8-expressing target cells were seeded into 384-well plates and left to attach for 16 hours. The next day, PBMC were added into the 384-well plates with the effector to target cell ratio (E:T) of 2:1. Then co-cultured cells were treated with a series dilution of BE-774 or BE-653 for 48 hours at 37° C. Culture supernatant was collected for subsequent measurement of IFN-7 and IL-2 concentration by a TR-FRET-based method (Degorce, Frangois, et al. Current chemical genomics. 2009, 3: 22) as described by the manufacturer manual (Cisbio). The results showed that anti-GPC3×CD137 bispecific antibodies including BE-774 and BE-653 induced dose-dependent cytokine release in PBMC cocultured with HepG2 cells but not with GPC3 negative cells (FIG. 5B). PBMCs from two donors were tested and results were shown in FIG. 5B.

### Example 8. Anti-GPC3×CD137 Enhances T Cell Killing Activity to GPC3 Positive Tumor Cells

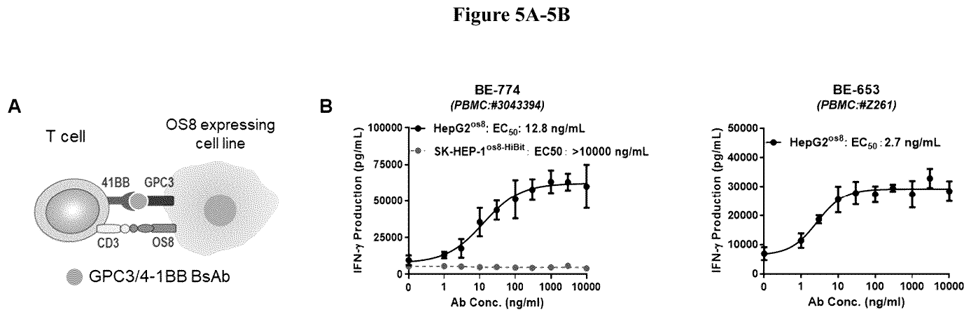

The ability of anti-GPC3×CD137 bispecific antibodies to induce T-cell killing activity was assessed in co-culture experiments with impedance measurements using an xCELLigence™ RTCA MP instrument (Agilent Technologies). Frozen human PBMC (AllCells) were thawed in RPMI 1640 medium and incubated at 37° C. overnight. Target cells were seeded into 96-well-E plates (Agilent Technologies) and left to attach for 16 hours. The next day, PBMC were added into the 96-well-E plates with the effector to target cell ratio (E:T) of 5:1. Then co-cultured cells were treated with a series dilution of BE-774 or BE-653 in combination with an EpCAM/CD3 bispecific T-cell engager (BiTE) which provides signal 1 for T cell activation (FIG. 6A). The experiment was allowed to proceed for 4 days to measure electrode impedance by live adherent target cells. The results showed that both BE-774 and BE-653 dose-dependently enhanced T cell killing activity to GPC3 expressing HepG2 cells but not to GPC3 negative SK-OV-3 cells (FIG. 6B).

### Example 9. Pharmacokinetic of BE-774 and BE-933 in Cynomolgus Monkeys

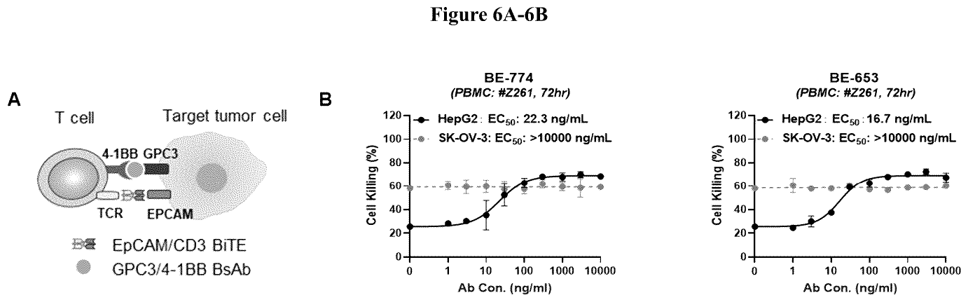

Blood samples were collected at 0, 0.00694 (10 minutes), 0.0417 (1 hour), 0.167 (4 hours), 0.333 (8 hours), 1, 4, 7, 10, 14, 21, and 28 days after 5 mg/kg intravenously administration of BE-774 or BE-933 from Cynomolgus monkeys, followed by centrifugation (4° C., 3500 xg, 2 min) to separate serum. The concentrations of BE-774 or BE-933 were measured by an in-house developed ELISA. Briefly, tagged GPC3 protein (Cat.: C414, Novoprotein, China) was used as a capture reagent, biotin labelled CD137 antigen (Cat.: 41B-H82E6, ACRO, China) was used as the detection reagent for BE-774 or BE-933 measurement. Pharmacokinetic (PK) profiles of BE-774 and BE-933 at the dosage levels of 5 mg/kg are shown in FIG. 7. PK parameters of BE-774 and BE-933 at the dosage levels of 5 mg/kg are shown in Table 9. BE-774 having the YTE mutation in the Fc showed significantly improved pharmacokinetics profile compared with BE-933.

TABLE 9
PK parameters of BE-774 and BE-933 in Cyno
PK Parameter after i.v 5 mg/kg in Cyno (n = 2)

| PK Parameter | BE-774 | BE-933 |
|---|---|---|
| T1/2 (day) | 4.80 | 1.92 |
| C0 (μg/mL) | 144 | 149 |
| Cmax (μg/mL) | 144 | 147 |
| AUClast (day*μg/mL) | 621 | 353 |
| AUCINF (day*μg/mL) | 795 | 372 |
| AUC_% Extrap (%) | 21.9 | 4.8 |
| Cl (mL/day/kg) | 6.29 | 37.3 |
| Vz (mL/kg) | 43.5 | 13.5 |
| MRTINF (day) | 6.59 | 2.64 |
| Vss (mL/kg) | 41.5 | 35.5 |

### Example 10. Pharmacokinetics of BE-774 and BE-933 in a hFcRn Mouse Model

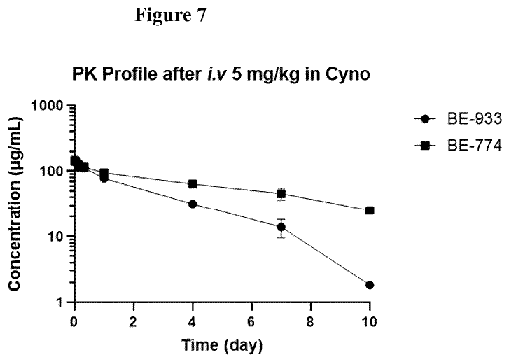

Blood samples were collected at 0, 0.0833 (2 hours), 1, 3, 7, 10, 14, 21, and 28 days after 3 mg/kg intravenously administration of BE-774 or BE-933 in a hFcRn mouse, followed by centrifugation (4dC, 3500 xg, 2 m) to separate serum. The concentrations of BE-774 or BE-933 were measured by an in-house developed ELISA. Briefly, tagged GPC3 protein (Cat.: C414, Novoprotein, China) was used as a capture reagent, biotin labelled CD137 antigen (Cat.: 41B-H82E6, ACRO, China) was used as the detection reagent for BE-774 or BE-933 measurement. Pharmacokinetic (PK) profiles of BE-774 and BE-933 at the dosage levels of 3 mg/kg are shown in FIG. 8. PK parameters of BE-774 and BE-933 at the dosage levels of 3 mg/kg are shown in Table 10. BE-933 had a favorable pharmacokinetics profile in hFcRn mouse, and BE-774 having the YTE mutation in the Fc showed significantly improved pharmacokinetics profile compared with BE-933.

TABLE 10
PK parameters of BE-774 and BE-933 in hFcRn mouse
PK Parameter after i.v 3 mg/kg in hFcRn Mouse (n = 3)

| PK Parameter | BE-774 | BE-933 |
|---|---|---|
| T1/2 (day) | 8.60 | 5.77 |
| C0 (μg/mL) | 79.2 | 65.9 |
| Cmax (μg/mL) | 74.8 | 61.4 |
| AUClast (day*μg/mL) | 569 | 304 |
| AUCINF (day*μg/mL) | 636 | 317 |
| AUC_% Extrap (%) | 9.40 | 3.74 |
| Cl (mL/day/kg) | 4.79 | 9.52 |
| Vz (mL/kg) | 57.0 | 78.2 |
| MRTINF (day) | 12.5 | 7.71 |
| Vss (mL/kg) | 57.6 | 72.6 |

### Example 11. SPR Binding of BE-915 to CD137 and GPC3

The binding kinetics of the antibodies were measured using surface plasmon resonance (SPR). SPR was used to measure the on-rate constant (Kₐ) and off-rate constant (K_{d}) of the antibodies to recombinant proteins of CD137 and GPC3 and then determined the affinity constant (K_{D}). The results show that BE-915 has good binding affinities to both human CD137 and human GPC3 (Table 11 and Table 12).

To test the binding specificity of BE-915 on CD137 from different species, SPR binding studies were performed using human CD137 (Cat.: 41B-H5256, Acrobio, CHINA) and cynomolgus monkey CD137 ECD (SEQ ID NO: 102) as bait proteins. BE-915 displayed a high binding affinity to human CD137, with a K_{D} of approximately 3.6 nM, which is similar to that to cynomolgus monkey CD137 (K_{D} of approximately 4.4 nM), as shown in Table 11. It also indicates the anti-CD137 antibody BGA-2524 used in BE-915 has great binding affinity to human CD137 and cynomolgus monkey CD137.

To test the binding specificity of BE-915 on GPC3 from different species, SPR binding studies were performed using human GPC3 (Cat.: 10088-H08H, Sino Biological, CHINA) and cynomolgus monkey GPC3 (Cat.: GP3-C5225, Acrobio, CHINA) as bait proteins. BE-915 displayed a binding affinity to human GPC3 with a K_{D} of approximately 2.4 nM and cynomolgus monkey GPC3 with a K_{D} of approximately 0.53 nM, as shown in Table 12.

TABLE 11
SPR binding of BE-915 to human and cyno CD137

| Antigen | Antibody | Kₐ (1/Ms) | K_{d} (1/s) | K_{D} (M) |
|---|---|---|---|---|
| Human CD137 | BE-915 | 1.2e+5 | 4.2e−4 | 3.6e−9 |
| Cyno CD137 | BE-915 | 1.3e+5 | 5.5e−4 | 4.4e−9 |

TABLE 12
SPR binding of BE-915 to human and cyno GPC3

| Antigen | Antibody | Kₐ (1/Ms) | K_{d} (1/s) | K_{D} (M) |
|---|---|---|---|---|
| Human GPC3 | BE-915 | 5.8e+4 | 1.4e−4 | 2.4e−9 |
| Cyno GPC3 | BE-915 | 2.1e+5 | 1.1e−4 | 5.3e−10 |

### Example 12. Binding of BE-915 to Native Human CD137 and Human GPC3

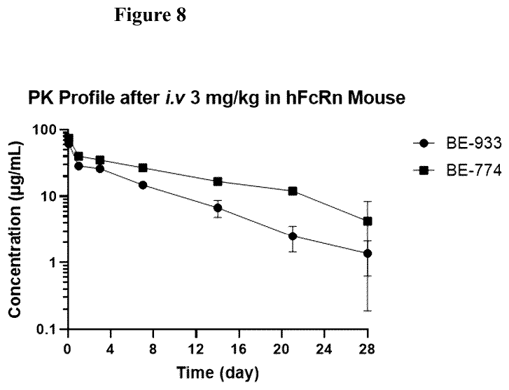

To validate the binding of BE-915 to native human CD137 and human GPC3 expressing on cells, human CD137-overexpressing HuT78 cells (HuT78/CD137) and HepG2 cells expressing native human GPC3 were used separately to evaluate the binding of BE-915. Fluorescence-activated cell sorting (FACS) demonstrated that BE-915 has strong binding activity to human CD137 and human GPC3 in a dose-dependent manner with an EC₅₀ of 0.69 nM (FIG. 9B) and 1.09 nM (FIG. 9A) respectively, as shown in FIG. 9. The isotype control hulgG (Cat.: 02-7102, Thermo, USA) showed no binding activity to HuT78/CD137 or HepG2 cells.

### Example 13. Binding Specificity of BE-915 with Other TNFRSF Members

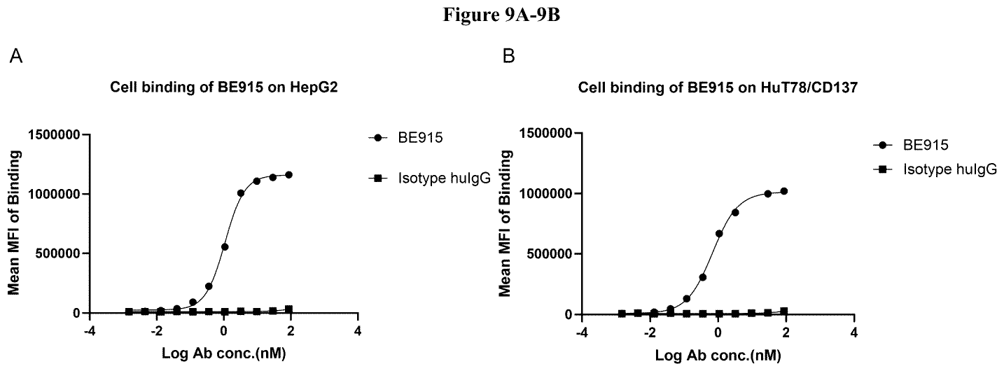

To test the binding specificity of BE-915 to other TNFRSF members, ELISA was performed by coating TNFRSF4/OX40 (Cat.: OXO-H5252, Acrobio, CHINA), TNFRSF7/CD27 (Cat.: CD7-H5257, Acrobio, CHINA), TNFRSF14/HVEM (Cat.: CD7-H522b, Acrobio, CHINA), TNFRSF8/CD30 (Cat.: HVM-H5258, Acrobio, CHINA) and human CD137 (Cat.: 41B-H5256, Acrobio, CHINA) on plate followed by binding with BE-915. As shown in FIG. 10, BE-915 specifically binds to human CD137 without binding to other TNFRSF members. It also indicates the anti-CD137 antibody BGA-2524 used in BE-915 specifically binds to human CD137.

### Example 14. BE-915 Competes with Human CD137L on Binding to Human CD137

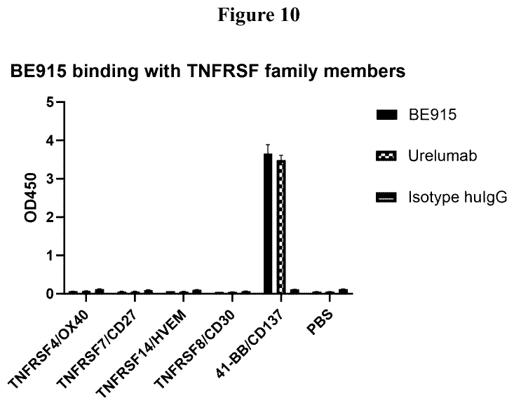

To accurately assess the blockade of BE-915 to CD137-CD137L binding at a cellular level, a cell-based blockade assay using HuT78/CD137 was established. The competitive blocking of CD137L against CD137 and BE-915 interaction was measured by detecting the binding of BE-915 (starting from the concentration of 15 μg/mL, followed by 3 fold series dilution) to human CD137 expressed on HuT78 in the presence of 10 μg/mL, 1 μg/mL, 0.1 μg/mL or 0 μg/mL CD137L (Cat.: 41L-H52D4, Acrobio, CHINA). Binding signal was detected by using secondary antibody anti-hFc 647 (Cat.: 109-605-098, Jackson, USA). hulgG (Cat.: 02-7102, Thermo, USA) was used as isotype control (FIG. 11A). As CD137L concentration increased, the binding of BE-915 to CD137 decreased (FIG. 11A). The competitive blocking of BE-915 against CD137 and CD137L interaction was measured by detecting the binding of CD137L (starting from the concentration of 15 μg/mL, followed by 3 fold series dilution) to CD137 expressed on HuT78 in the presence of 1 μg/mL, 0.1 μg/mL or 0 μg/mL BE-915. Binding signal was detected by using secondary antibody anti-his 647 (Cat.: A01802, Genscript, CHINA). hulgG (Cat.: 02-7102, Thermo, USA) was used as isotype control (FIG. 11B). As BE-915 concentration increased, the binding of CD137L to CD137 decreased (FIG. 11B). These data demonstrate that BE-915 cross-competes with CD137L on binding to human CD137. It also indicates the anti-CD137 antibody BGA-2524 used in BE-915 cross-competes with CD137L on binding to human CD137.

### Example 15. BE-915 Induces T Cell Activation Cocultured with GPC3 Positive Tumor Cells

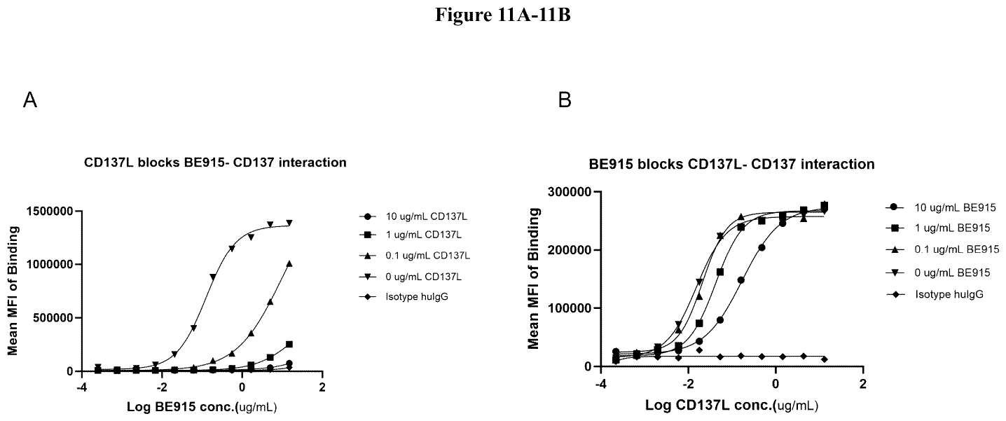

The functional activity of GPC3×CD137 bispecific antibody BE-915 was assessed in in vitro co-culture experiments using human peripheral blood mononuclear cell (PBMC) and OS8-expressing hepatocellular carcinoma (HCC) cell lines (FIG. 12A). Three HCC cell lines, HepG2, Huh7 and Hep3B, with high to low GPC3 expression based on the FACS analysis (FIG. 12B) were chosen to evaluate the effect of GPC3 levels on the functional activity of BE-915. No GPC3 expressing SK-HEP-1 was used as a negative control cell line.

Frozen human PBMC (OriBiotech) were thawed in RPMI 1640 medium and incubated at 37° C. overnight. OS8-expressing target cells were seeded into 384-well plates and left to attach for 16 hours. The next day, PBMC were added into the 384-well plates with the effector to target cell ratio (E:T) of 2:1. Then co-cultured cells were treated with a series dilution of BE-915 for 48 hours at 37° C. Culture supernatant was collected for subsequent measurement of IFN-7 and IL-2 concentration by a TR-FRET-based method (Degorce, Frangois, et al. Current Chemical Genomics. 2009, 3: 22) as described by the manufacturer manual (Cisbio). The results showed that BE-915 induced dose-dependent cytokine release in PBMC from two independent donors cocultured with GPC3 expressing cells but not with GPC3 negative cells (FIG. 12C).

### Example 16. BE-915 Enhances PBMC Based Cell Killing Cultured with GPC3 Positive Tumor Cells

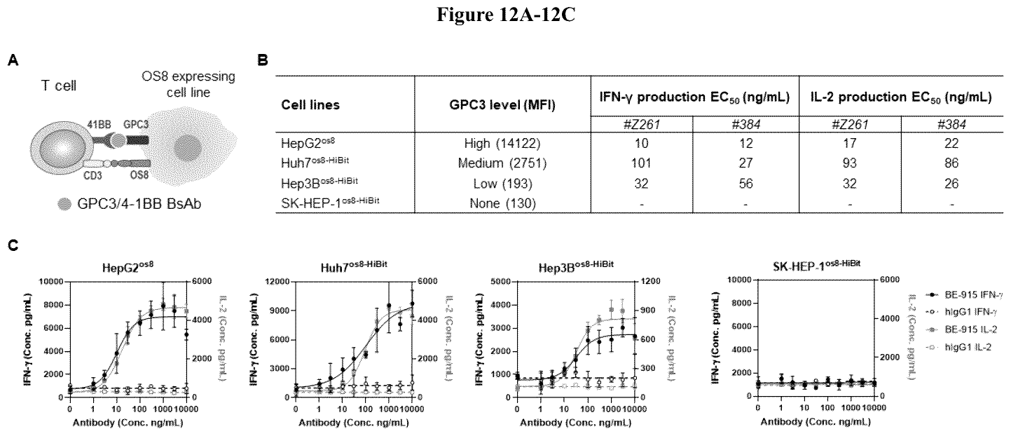

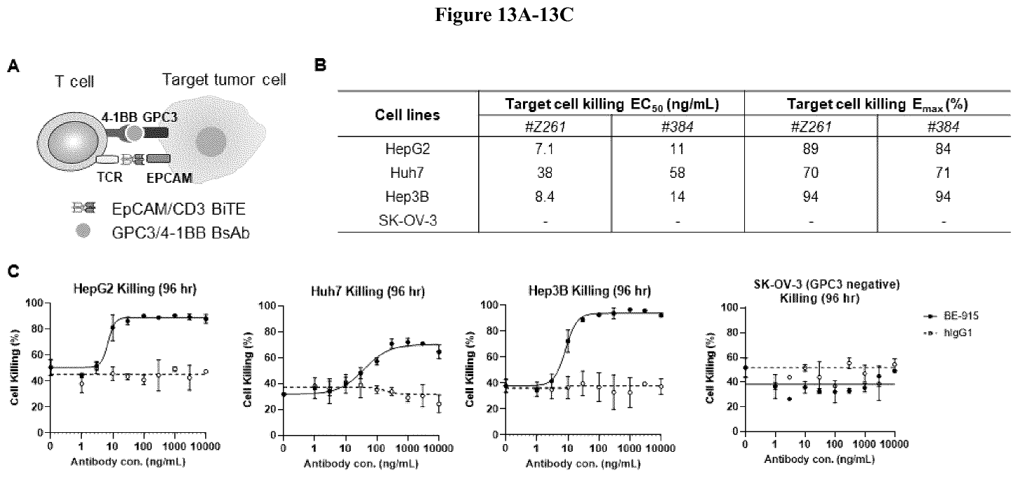

BE-915 regulated T-cell killing activity was assessed in co-culture experiments with impedance measurements using an xCELLigence RTCA MP instrument (Agilent Technologies). Frozen human PBMC (OriBiotech) were thawed in RPMI 1640 medium and incubated at 37° C. overnight. Target cells were seeded into 96-well-E plates (Agilent Technologies) and left to attach for 16 hours. The next day, PBMC were added into the 96-well-E plates with the effector to target cell ratio (E:T) of 5:1. Then co-cultured cells were treated with a series dilution of BE-915 in combination with an EpCAM/CD3 bispecific T-cell engager (BiTE) which provides signal 1 for T cell activation (FIG. 13A). Three HCC cell lines, HepG2, Huh7 and Hep3B, with high to low GPC3 expression based on the FACS analysis (FIG. 13B) were chosen to evaluate the effect of GPC3 levels on the functional activity of BE-915. No GPC3 expressing SK-OV-3 was used as a negative control cell line.

The experiment was allowed to proceed for 4 days to measure electrode impedance by live adherent target cells. Consistent with cytokine production assay, BE-915 dose-dependently enhanced T cell killing activity to GPC3 expressing cells but not to GPC3 negative cells (FIG. 13C). PBMCs from two donors were used in this experiment.

### Example 17. Pharmacokinetics Profile of BE-915 in Cynomolgus

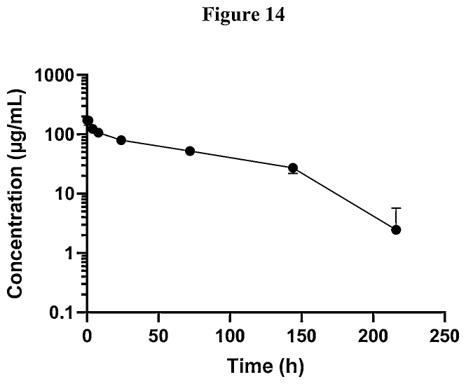

Blood samples were collected from Cynomolgus at 0, 0.167 hours, 1 hours, 4 hours, 8 hours, 1, 3, 6, 9, 13, 20 and 27 days after 5 mg/kg intravenously infusion of BE-915, followed by centrifugation (4° C., 3000 xg, 15 min) to separate serum. The concentrations of BE-915 were measured by in-house developed ELISA ligand binding methods. Briefly, tagged GPC3 antigen (Cat.: C414, Novoprotein, China) was used as a capture reagent, biotin labelled CD137 antigen (Cat.: 41B-H82E6, ACRO, China) was used as the detection reagents for BE-915. The obtained pharmacokinetics profiles and parameters are shown in FIG. 14 and Table 13, respectively. In the 5 mg/kg dosing group, BE-915 was below the lower limit of quantification (0.0391 g/mL) on day 13 post dose. Anti-drug antibody (ADA) was detected in serum since day 9 in 5 mg/kg dosing group, which indicated a potential influence on the pharmacokinetic curve. The PK parameters is calculated after removing the concentration value of the time point. The clearance of BE-915 is 10.4 mL/day/kg, with an antibody-like half-life of 3.2 day.

TABLE 13
Pharmacokinetics parameters of BE-915 in Cynomolgus after i.v infusion
PK Parameter after i.v infusion of BE-915 in Cyno (n = 2)

| PK Parameter | 5 mg/kg |
|---|---|
| T1/2 (day) | 3.2 |
| Cmax (μg/mL) | 127 |
| AUClast (day*μg/mL) | 358 |
| AUCINF (day*μg/mL) | 485 |
| AUC_% Extrap (%) | 26 |
| CL (mL/day/kg) | 10.4 |
| Vss (mL/kg) | 44.7 |

Note:
non-compartment model was used to calculate the pharmacokinetics parameters

### Example 18. Efficacy of BE-915 Monotherapy in MC38/hGPC3 Model in Humanized CD137 Knock-In Mice

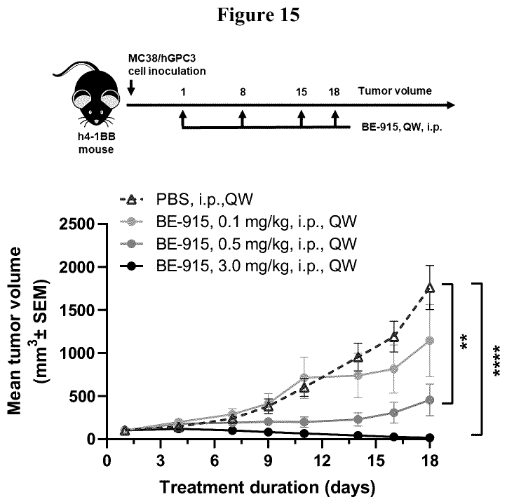

The in vivo efficacy of BE-915 was examined in the MC38/hGPC3 mouse colorectal carcinoma model in humanized CD137 knock-in mice. MC38/hGPC3 cells were subcutaneously implanted into the right flank of the recipient mice. Seven days after cell inoculation, the mice were randomized into 4 groups according to tumor volume. BE-915 was intraperitoneally administrated on Day 1 and were administrated once weekly for 18 days. BE-915 (0.1, 0.5, and 3.0 mg/kg, once weekly) effectively inhibited tumor growth. The tumor volume was significantly decreased at study endpoint (D18). In addition, the ratios of tumor free in 0.1, 0.5 and 3.0 mg/kg groups was 0%, 0%, and 10% on Day 18, respectively (FIG. 15 and Table 14). There was no significant impact on animal body weight in any of the treatment group throughout the study.

TABLE 14
Efficacy of BE-915 in the MC38/hGPC3 Syngeneic
Tumor Model in Humanized CD137 Knock-in Mice

| | | | Mean tumor | | P-value vs | | | |
|---|---|---|---|---|---|---|---|---|
| Test | | Dose | volume on Day 18 | | vehicle on | TGI rate on | Ratio of tumor | |
| article | n | (mg/kg) | (mm3 ± SEM) | | Day 18 | Day 18 (%) | free on Day 18 | |
| Vehicle | 10 | NA | 1761.1 | (±90.5) | NA | NA | 0% | (0/10) |
| BE-915 | 10 | 0.1, QW | 1145.1 | (±148.3) | 0.2807 | 37.1 | 0% | (0/10) |
| BE-915 | 10 | 0.5, QW | 455.2 | (±57.6) | 0.0019 | 78.8 | 0% | (0/10) |
| BE-915 | 10 | 3.0, QW | 14.6 | (±2.1) | <0.0001 | 105.3 | 10% | (1/10) |

Abbreviations: hGPC3, human Glypican 3; n, number of animals; NA, not applicable; QW, once weekly; SEM, standard error of the mean; TGI, tumor growth inhibition.
Note:
TGI rate was calculated according to the following formula: % TGI = [1 − (treated Tt − treated T0)/(vehicle Tt − vehicle T0)] × 100%. Treated Tt = mean tumor volume of a dosing group on Day t; treated T0 = mean tumor volume of a dosing group on Day 0; vehicle Tt = mean tumor volume of vehicle group on Day t; and vehicle T0 = mean tumor volume of vehicle group on Day 0.

### Example 19. Efficacy of the Combination of BE-915 and Anti-PD-1 Antibody in LL/2/hGPC3 Model in Humanized 4-1BB Knock-In Mice

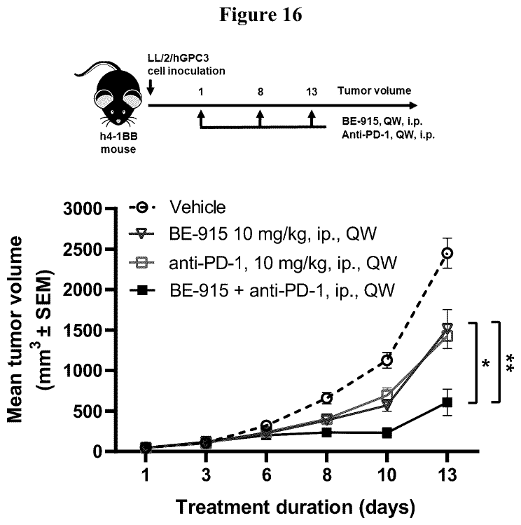

The antitumor activity of the combination of BE-915 and anti-mouse PD-1 antibody was investigated in the LL/2/hGPC3 syngeneic model (lung cancer) in humanized CD137 knock-in mice. LL/2/hGPC3 cells were implanted into the mice. Seven days after cell inoculation, the mice were randomized into 4 groups according to tumor volume. Mice receiving the combination treatment of BE-915 (10.0 mg/kg, once weekly) and anti-mouse PD-1 antibody (10.0 mg/kg, once weekly) exhibited synergistic tumor growth inhibition. The tumor growth inhibition rate in the combination group was 74.7% on Day 13, which was significantly higher than that in the group treated with BE-915 (34.1%) or anti-PD-1 (38.0%) alone (FIG. 16 and Table 15). No significant impact on animal body weight was observed in any treatment group throughout the study.

TABLE 15
Antitumor Effect of BE-915 and anti-mouse PD-1 antibody in
LL/2/hGPC3 Syngeneic Model in Humanized CD137 Knock-in Mice

| | | | | | P value vs. |
|---|---|---|---|---|---|
| | | | | Mean tumor | BE-915 + |
| | | | TGI rate | volume on | anti-PD-1 |
| Test | | Dosage | on Day | Day 13 | group on |
| article | n | (mg/kg) | 13 (%) | (mm³ ± SEM) | Day 13 |
| Vehicle | 10 | NA | NA | 2272.6 (±75.0) | NA |
| BE-915 | 10 | 10.0 QW | 34.1 | 1512.0 (±76.2) | 0.0142 |
| Anti-PD-1 | 10 | 10.0 QW | 38.0 | 1424.6 (±49.5) | 0.0082 |
| BE-915 + | 10 | 10.0 + | 74.7 | 606.9 (±51.3) | NA |
| anti-PD-1 | | 10.0 QW | | | |

Abbreviations: hGPC3, human Glypican 3; n, number of animals; NA, not applicable; QW, once weekly; SEM, standard error of the mean; TGI, tumor growth inhibition.
Note:
TGI rate was calculated according to the following formula: % TGI = [1 − (treated Tt − treated T0) / (vehicle Tt − vehicle T0)] × 100%. Treated Tt = mean tumor volume of a dosing group on Day t; treated T0 = mean tumor volume of a dosing group on Day 0; vehicle Tt = mean tumor volume of vehicle group on Day t; and vehicle T0 = mean tumor volume of vehicle group on Day 0.

### Example 20. Biophysical Properties of BE-915

The biophysical properties of BE-774 (using camelid CD137 VHH BGA-9612) and BE-915 (using humanized CD137 VHH BGA-2524) were tested. The tested biophysical properties included melting temperature, aggregation temperature, hydrophobicity by HIC-HPLC and self-association tendency by AC-SINS (see below the detailed description). BE-915 showed optimal thermal stability by Tm and Tagg and good colloidal stability by AC-SINS. As shown in Table 16, BE-915 showed comparable overall biophysical property with BE-774. It also indicates that humanized CD137 VHH BGA-2524 used in BE-915 has comparable overall biophysical property with camelid CD137 VHH BGA-9612 used in BE-774.

TABLE 16
Summary of biophysical properties of BE-915 and BE-774

| | Tm | Tagg | HIC Retention | AC-SINS |
|---|---|---|---|---|
| Antibody | (° C.) | (° C.) | Time (min) | Δλ (nm) |
| BE-774 | 64.53 | 70.66 | 21.61 | 0 |
| BE-915 | 64.28 | 70.35 | 22.15 | 1.7 |

Melting temperature (Tm) and the aggregation temperature Tagg (° C.) were determined by the UNCLE™ (Unchained lab, Pleasanton, CA), which was an instrument measuring the intrinsic fluorescence and static light scattering simultaneously. During the measurement, 9 μL protein sample at 1 mg/mL in PBS buffer was loaded to the cuvette; the samples were held at 20° C. for 120 s and then ramped to 95° C. at the rate of 0.3° C./min. Both fluorescence and static light scattering (at 266 nm) were collected after excitation at 266 nm.

To determine the hydrophobicity of a given antibody using HPLC system, 50 g of sample at 1 mg/ml was diluted with a mobile phase A solution (1.5 M ammonium sulfate, 50 mM sodium phosphate, pH 7.0) to achieve a final ammonium sulfate concentration of about 1M before analysis. A MABPac HIC-10 column was used with a liner gradient of mobile phase A and mobile phase B solution (50 mM sodium phosphate, pH 7.0) over 29 min at a flow rate of 0.5 mg/min. Peak retention times were monitored at A280 absorbance.

The AC-SINS assay measures protein self-interaction by capturing the antibody on the surface of a gold colloid that displays surface resonance oscillations in frequency with visible light. As the immobilized antibodies self-interact, the colloids aggregate, changing the oscillation frequency to absorb at a longer wavelength. Gold nanoparticles were incubated with an 80/20 (v/v) capture antibody/non-capture antibody mixture. The coated gold nanoparticles were then spun down and resuspended into PBS. The samples were diluted to 0.05 mg/ml into the conjugation buffers and 45 μl of each dilution was loaded onto a 384-well plate. Five μl of previously prepared gold nanoparticles were then added to each well of the plate including mAbs and buffer controls. The plate was then covered with an aluminum lid, incubated at room temperature for 2 hours, and quickly spun down at 3000 rpm prior to reading the absorbance spectra of each well from 450 to 650 nm using a plate reader. Each sample spectra were recorded and analyzed for red-shifting of the maximum of absorption peak compared to buffers, the red-shifting and its strength is indicative of the self-interaction propensity of the tested mAb sample.

The amino acid and DNA sequences of anti-CD137 VHHs and anti-GPC3×CD137 bispecific antibodies are shown in Table 17 below.

TABLE 17
Amino acid and DNA sequences of anti-CD137 VHHs and anti-GPC3xCD137
bispecific antibodies

| Antibody | SEQ ID NO | | SEQUENCE |
|---|---|---|---|
| BGA-9612 | SEQ ID NO: 1 | CDR1 | NYAVG |
| | | (Kabat) | |
| | SEQ ID NO: 2 | CDR2 | TYWSGYAHYGDFENN |
| | | (Kabat) | |
| | SEQ ID NO: 3 | CDR3 | SLKYPMATVTPDY |
| | | (Kabat) | |
| | SEQ ID NO: 4 | VHH AA | QVQLVESGGGTVQPGDSLRLSCAASGRTFDNYAVGWF |
| | | | RQAPGKERQYVGTYWSGYAHYGDFENNRFTISRDNAK |
| | | | NTVYLQMDNLKPEDTAVYYCAISLKYPMATVTPDYQG |
| | | | QGTQVTVSS |
| | SEQ ID NO: 5 | VHH DNA | CAGGTGCAGCTCGTGGAGTCCGGGGGAGGAACGGTG |
| | | | CAGCCTGGGGACTCTCTGAGACTCTCCTGTGCTGCCT |
| | | | CTGGACGCACCTTCGATAACTATGCCGTGGGCTGGTT |
| | | | CCGCCAGGCTCCAGGGAAGGAGCGTCAGTATGTCGG |
| | | | CACGTATTGGAGTGGTTACGCACACTATGGGGACTTT |
| | | | GAGAATAACCGATTCACGATCTCCAGAGACAACGCCA |
| | | | AGAATACGGTGTATCTGCAAATGGACAACCTGAAACC |
| | | | TGAGGACACGGCCGTTTATTACTGTGCAATATCCCTTA |
| | | | AATATCCTATGGCGACAGTTACCCCAGACTACCAGGG |
| | | | CCAGGGGACCCAGGTCACCGTCTCCTCA |
| BGA-6582 | SEQ ID NO: 1 | CDR1 | NYAVG |
| | | (Kabat) | |
| | SEQ ID NO: 2 | CDR2 | TYWSGYAHYGDFENN |
| | | (Kabat) | |
| | SEQ ID NO: 3 | CDR3 | SLKYPMATVTPDY |
| | | (Kabat) | |
| | SEQ ID NO: 8 | VHH AA | EVQLLESGGGLVQPGGSLRLSCAASGRTFDNYAVGWFR |
| | | | QAPGKERQYVGTYWSGYAHYGDFENNRFTISRDNSKN |
| | | | TVYLQMNSLRAEDTAVYYCAISLKYPMATVTPDYQGQ |
| | | | GTLVTVSS |
| | SEQ ID NO: 9 | VHH DNA | GAAGTGCAGCTGCTGGAGTCCGGGGGCGGCCTGGTC |
| | | | CAACCCGGGGGCAGCCTGAGACTGAGCTGCGCCGCC |
| | | | TCCGGCAGAACCTTCGACAACTACGCCGTGGGCTGG |
| | | | TTCAGACAAGCCCCCGGCAAGGAGAGACAGTACGTG |
| | | | GGCACCTACTGGAGCGGCTACGCCCACTACGGCGAC |
| | | | TTCGAGAACAACAGATTCACCATCAGCAGAGACAAC |
| | | | AGCAAGAACACCGTGTACCTGCAGATGAACAGCCTG |
| | | | AGAGCCGAGGACACCGCCGTGTACTACTGCGCCATC |
| | | | AGCCTGAAGTACCCCATGGCCACCGTGACCCCCGAC |
| | | | TACCAAGGCCAAGGCACCCTGGTGACCGTGAGCAGC |
| BGA-3726 | SEQ ID NO: 1 | CDR1 | NYAVG |
| | | (Kabat) | |
| | SEQ ID NO: 2 | CDR2 | TYWSGYAHYGDFENN |
| | | (Kabat) | |
| | SEQ ID NO: 3 | CDR3 | SLKYPMATVTPDY |
| | | (Kabat) | |
| | SEQ ID NO: 6 | VHH AA | EVQLLESGGGLVQPGGSLRLSCAASGRTFDNYAVGWF |
| | | | RQAPGKEREYVGTYWSGYAHYGDFENNRFTISRDNSK |
| | | | NTVYLQMNSLRAEDTAVYYCAISLKYPMATVTPDYQG |
| | | | QGTLVTVSS |
| | SEQ ID NO: 7 | VHH DNA | GAAGTGCAGCTGCTGGAGTCCGGGGGCGGCCTGGTC |
| | | | CAACCCGGGGGCAGCCTGAGACTGAGCTGCGCCGCC |
| | | | TCCGGCAGAACCTTCGACAACTACGCCGTGGGCTGG |
| | | | TTCAGACAAGCCCCCGGCAAGGAGAGAGAGTACGTG |
| | | | GGCACCTACTGGAGCGGCTACGCCCACTACGGCGAC |
| | | | TTCGAGAACAACAGATTCACCATCAGCAGAGACAAC |
| | | | AGCAAGAACACCGTGTACCTGCAGATGAACAGCCTG |
| | | | AGAGCCGAGGACACCGCCGTGTACTACTGCGCCATC |
| | | | AGCCTGAAGTACCCCATGGCCACCGTGACCCCCGAC |
| | | | TACCAAGGCCAAGGCACCCTGGTGACCGTGAGCAGC |
| BGA-3544 | SEQ ID NO: 1 | CDR1 | NYAVG |
| | | (Kabat) | |
| | SEQ ID NO: 10 | CDR2 | TYWSGYAHYGDFEKG |
| | | (Kabat) | |
| | SEQ ID NO: 3 | CDR3 | SLKYPMATVTPDY |
| | | (Kabat) | |
| | SEQ ID NO: 11 | VHH AA | EVQLVESGGGLVQPGGSLRLSCAASGRTFDNYAVGWFR |
| | | | QAPGKEREYVGTYWSGYAHYGDFEKGRFTISRDNSKN |
| | | | TVYLQMNNLRPEDTAVYYCAISLKYPMATVTPDYQGQ |
| | | | GTQVTVSS |
| | SEQ ID NO: 12 | VHH DNA | GAAGTGCAGCTGGTGGAGTCCGGGGGCGGCCTGGTC |
| | | | CAACCCGGGGGCAGCCTGAGACTGAGCTGCGCCGCC |
| | | | TCCGGCAGAACCTTCGACAACTACGCCGTGGGCTGG |
| | | | TTCAGACAAGCCCCCGGCAAGGAGAGAGAGTACGTG |
| | | | GGCACCTACTGGAGCGGCTACGCCCACTACGGCGAC |
| | | | TTCGAGAAGGGCAGATTCACCATCAGCAGAGACAAC |
| | | | AGCAAGAACACCGTGTACCTGCAGATGAACAACCTG |
| | | | AGACCCGAGGACACCGCCGTGTACTACTGCGCCATC |
| | | | AGCCTGAAGTACCCCATGGCCACCGTGACCCCCGAC |
| | | | TACCAAGGCCAAGGCACCCAAGTGACCGTGAGCAGC |
| BGA-9502 | SEQ ID NO: 13 | VHH AA | EVQLVESGGGLVQPGGSLRLSCAASGRTFDNYAVGWFR |
| | | | QAPGKEREYVGTYWSGYAHYGDFEKGRFTISRDNAKN |
| | | | TVYLQMNNLRPEDTAVYYCAISLKYPMATVTPDYQGQ |
| | | | GTQVTVSS |
| | SEQ ID NO: 14 | VHH DNA | GAAGTGCAGCTGGTGGAGTCCGGGGGCGGCCTGGTC |
| | | | CAACCCGGGGGCAGCCTGAGACTGAGCTGCGCCGCC |
| | | | TCCGGCAGAACCTTCGACAACTACGCCGTGGGCTGG |
| | | | TTCAGACAAGCCCCCGGCAAGGAGAGAGAGTACGTG |
| | | | GGCACCTACTGGAGCGGCTACGCCCACTACGGCGAC |
| | | | TTCGAGAAGGGCAGATTCACCATCAGCAGAGACAAC |
| | | | GCCAAGAACACCGTGTACCTGCAGATGAACAACCTG |
| | | | AGACCCGAGGACACCGCCGTGTACTACTGCGCCATC |
| | | | AGCCTGAAGTACCCCATGGCCACCGTGACCCCCGAC |
| | | | TACCAAGGCCAAGGCACCCAAGTGACCGTGAGCAGC |
| BGA-7031 | SEQ ID NO: 1 | CDR1 | NYAVG |
| | | (Kabat) | |
| | SEQ ID NO: 2 | CDR2 | TYWSGYAHYGDFENN |
| | | (Kabat) | |
| | SEQ ID NO: 3 | CDR3 | SLKYPMATVTPDY |
| | | (Kabat) | |
| | SEQ ID NO: 15 | VHH AA | EVQLVESGGGLVQPGGSLRLSCAASGRTFDNYAVGWF |
| | | | RQAPGKEREYVGTYWSGYAHYGDFENNRFTISRDNSK |
| | | | NTVYLQMNNLRPEDTAVYYCAISLKYPMATVTPDYQG |
| | | | QGTQVTVSS |
| | SEQ ID NO: 16 | VHH DNA | GAAGTGCAGCTGGTGGAGTCCGGGGGCGGCCTGGTC |
| | | | CAACCCGGGGGCAGCCTGAGACTGAGCTGCGCCGCC |
| | | | TCCGGCAGAACCTTCGACAACTACGCCGTGGGCTGGT |
| | | | TCAGACAAGCCCCCGGCAAGGAGAGAGAGTACGTGG |
| | | | GCACCTACTGGAGCGGCTACGCCCACTACGGCGACTT |
| | | | CGAGAACAACAGATTCACCATCAGCAGAGACAACAG |
| | | | CAAGAACACCGTGTACCTGCAGATGAACAACCTGAG |
| | | | ACCCGAGGACACCGCCGTGTACTACTGCGCCATCAGC |
| | | | CTGAAGTACCCCATGGCCACCGTGACCCCCGACTACC |
| | | | AAGGCCAAGGCACCCAAGTGACCGTGAGCAGC |
| BGA-2524 | SEQ ID NO: 17 | VHH AA | EVQLVESGGGLVQPGGSLRLSCAASGRTFDNYAVGWF |
| | | | RQAPGKEREYVGTYWSGYAHYGDFENNRFTISRDNAK |
| | | | NTVYLQMNNLRPEDTAVYYCAISLKYPMATVTPDYQG |
| | | | QGTQVTVSS |
| | SEQ ID NO: 18 | VHH DNA | GAAGTGCAGCTGGTGGAGTCCGGGGGCGGCCTGGTC |
| | | | CAACCCGGGGGCAGCCTGAGACTGAGCTGCGCCGCT |
| | | | AGCGGCAGAACCTTCGACAACTACGCCGTGGGCTGG |
| | | | TTCAGACAAGCCCCCGGCAAGGAGAGAGAGTACGTG |
| | | | GGCACCTACTGGAGCGGCTACGCCCACTACGGCGACT |
| | | | TCGAGAACAACAGATTCACCATCAGCAGAGACAACG |
| | | | CCAAGAACACCGTGTACCTGCAGATGAACAACCTGA |
| | | | GACCCGAGGACACCGCCGTGTACTACTGCGCCATCAG |
| | | | CCTGAAGTACCCCATGGCCACCGTGACCCCCGACTAC |
| | | | CAAGGCCAAGGCACCCAAGTGACCGTGAGCAGC |
| IgG1mf Fc | SEQ ID NO: 19 | Fc of | EPKSSDKTHTCPPCPAPPAAGPSVFLFPPKPKDTLMISRT |
| | | human | PEVTCVVVDVSHEDPEVKFNWYVDGVEVHNAKTKPR |
| | | IgG1 mf | EEQYNSTYRVVSVLTVLHQDWLNGKEYKCKVSNKALA |
| | | AA | APIEKTISKAKGQPREPQVYTLPPSREEMTKNQVSLTCL |
| | | | VKGFYPSDIAVEWESNGQPENNYKTTPPVLDSDGSFFLY |
| | | | SKLTVDKSRWQQGNVFSCSVMHEALHNHYTQKSLSLS |
| | | | PGK |
| IgG1mf Fc | SEQ ID NO: 53 | Fc of | EPKSCDKTHTCPPCPAPPAAGPSVFLFPPKPKDTLMISRT |
| | | human | PEVTCVVVDVSHEDPEVKFNWYVDGVEVHNAKTKPR |
| | | IgG1 mf | EEQYNSTYRVVSVLTVLHQDWLNGKEYKCKVSNKALA |
| | | AA | APIEKTISKAKGQPREPQVYTLPPSREEMTKNQVSLTCL |
| | | | VKGFYPSDIAVEWESNGQPENNYKTTPPVLDSDGSFFLY |
| | | | SKLTVDKSRWQQGNVFSCSVMHEALHNHYTQKSLSLS |
| | | | PGK |
| IgG1mf Fc- | SEQ ID NO: 20 | Fc of | EPKSCDKTHTCPPCPAPPAAGPSVFLFPPKPKDTLYITRE |
| YTE | | human | PEVTCVVVDVSHEDPEVKFNWYVDGVEVHNAKTKPR |
| | | IgG1 mf | EEQYNSTYRVVSVLTVLHQDWLNGKEYKCKVSNKALA |
| | | AA with | APIEKTISKAKGQPREPQVYTLPPSREEMTKNQVSLTCL |
| | | YTE | VKGFYPSDIAVEWESNGQPENNYKTTPPVLDSDGSFFLY |
| | | mutation | SKLTVDKSRWQQGNVFSCSVMHEALHNHYTQKSLSLS |
| | | | PGK |
| BE-915 | SEQ ID NO: 21 | VH | QVQLVQSGAEVKKPGASVKVSCKASGYTFTDYEMHW |
| | | | VRQAPGQGLEWMGALDPKTGDTAYSQKFKGRVTLTAD |
| | | | KSTSTAYMELSSLTSEDTAVYYCTRFYSYTYWGQGTLV |
| | | | TVSSASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPC |
| | | | PAPPAAGPSVFLFPPKPKDTLYITREPEVTCVVVDVSHE |
| | | | DPEVKFNWYVDGVEVHNAKTKPREEQYNSTYRVVSVL |
| | | | TVLHQDWLNGKEYKCKVSNKALAAPIEKTISKAKGQP |
| | | | REPQVYTLPPSREEMTKNQVSLTCLVKGFYPSDIAVEWE |
| | | | SNGQPENNYKTTPPVLDSDGSFFLYSKLTVDKSRWQQG |
| | | | NVFSCSVMHEALHNHYTQKSLSLSPGKGGGGSEVQLV |
| | | | ESGGGLVQPGGSLRLSCAASGRTFDNYAVGWFRQAPGK |
| | | | EREYVGTYWSGYAHYGDFENNRFTISRDNAKNTVYLQ |
| | | | MNNLRPEDTAVYYCAISLKYPMATVTPDYQGQGTQVT |
| | | | VSS |
| | SEQ ID NO: 22 | VH DNA | CAAGTGCAGCTGGTGCAGAGCGGCGCCGAGGTGAAG |
| | | | AAGCCCGGCGCTAGCGTGAAGGTGAGCTGCAAGGCT |
| | | | AGCGGCTACACCTTCACCGACTACGAAATGCACTGGG |
| | | | TCAGACAAGCCCCCGGCCAAGGCCTGGAATGGATGG |
| | | | GCGCCCTGGACCCCAAGACCGGCGACACCGCCTACA |
| | | | GCCAAAAGTTCAAGGGCAGAGTGACCCTGACCGCCG |
| | | | ACAAGAGCACAAGCACCGCCTACATGGAGCTGAGCA |
| | | | GCCTGACAAGCGAGGACACCGCCGTCTACTACTGCA |
| | | | CAAGATTCTACAGCTACACCTACTGGGGCCAAGGCAC |
| | | | CCTCGTGACCGTGAGCTCCGCTAGCACAAAGGGCCCT |
| | | | AGCGTGTTCCCCCTCGCTCCTAGCAGCAAGAGCACCT |
| | | | CCGGGGGCACAGCCGCCCTGGGCTGCCTGGTGAAGG |
| | | | ACTACTTCCCCGAGCCCGTGACCGTGAGCTGGAACA |
| | | | GCGGCGCCCTCACAAGCGGCGTGCATACCTTCCCCGC |
| | | | CGTGCTGCAGAGCAGCGGCCTGTACAGCCTGAGCAG |
| | | | CGTGGTGACCGTGCCTAGCAGCAGCCTGGGCACACA |
| | | | GACCTACATCTGCAACGTGAACCACAAGCCTAGCAAC |
| | | | ACCAAGGTGGACAAGAAGGTGGAGCCCAAGTCCTGT |
| | | | GATAAGACCCACACATGCCCTCCCTGCCCCGCCCCTC |
| | | | CCGCCGCCGGCCCTTCCGTGTTCCTGTTCCCCCCCAA |
| | | | GCCCAAGGACACCCTGTACATCACCAGAGAGCCCGA |
| | | | GGTGACCTGCGTGGTCGTGGACGTGAGCCACGAGGA |
| | | | CCCCGAGGTGAAGTTCAACTGGTACGTGGACGGCGT |
| | | | GGAGGTGCACAACGCCAAGACCAAGCCTAGAGAGGA |
| | | | GCAGTACAACAGCACCTACAGAGTGGTGAGCGTGCT |
| | | | GACCGTGCTGCACCAAGACTGGCTGAACGGCAAGGA |
| | | | GTACAAGTGCAAGGTGAGCAACAAGGCCCTGGCCGC |
| | | | CCCCATCGAGAAGACCATCAGCAAGGCCAAGGGGCA |
| | | | GCCTAGAGAGCCCCAAGTGTACACCCTGCCCCCTAGC |
| | | | AGAGAGGAGATGACCAAGAACCAAGTGAGCCTGACC |
| | | | TGCCTCGTGAAGGGCTTCTACCCTAGCGACATCGCCG |
| | | | TGGAGTGGGAGAGCAACGGGCAGCCCGAGAACAAC |
| | | | TACAAGACCACCCCCCCCGTGCTGGACAGCGACGGC |
| | | | AGCTTCTTCCTGTACAGCAAGCTGACCGTGGACAAGA |
| | | | GCAGATGGCAGCAAGGCAACGTGTTCAGCTGCAGCG |
| | | | TGATGCACGAGGCCCTGCACAACCACTACACCCAAA |
| | | | AAAGCCTCAGCCTCAGCCCCGGCAAGGGCGGGGGCG |
| | | | GCTCCGAAGTGCAGCTGGTGGAGTCCGGGGGCGGCC |
| | | | TGGTCCAACCCGGGGGCAGCCTGAGACTGAGCTGCG |
| | | | CCGCTAGCGGCAGAACCTTCGACAACTACGCCGTGG |
| | | | GCTGGTTCAGACAAGCCCCCGGCAAGGAGAGAGAGT |
| | | | ACGTGGGCACCTACTGGAGCGGCTACGCCCACTACG |
| | | | GCGACTTCGAGAACAACAGATTCACCATCAGCAGAG |
| | | | ACAACGCCAAGAACACCGTGTACCTGCAGATGAACA |
| | | | ACCTGAGACCCGAGGACACCGCCGTGTACTACTGCG |
| | | | CCATCAGCCTGAAGTACCCCATGGCCACCGTGACCCC |
| | | | CGACTACCAAGGCCAAGGCACCCAAGTGACCGTGAG |
| | | | CAGC |
| | SEQ ID NO: 23 | VLAA | DVVMTQSPLSLPVTPGEPASISCRSSQSLVHSNRNTYLH |
| | | | WYLQKPGQSPQLLIYKVSNRFSGVPDRFSGSGSGTDFT |
| | | | LKISRVEAEDVGVYYCSQNTHVPPTFGQGTKLEIKRTVA |
| | | | APSVFIFPPSDEQLKSGTASVVCLLNNFYPREAKVQWK |
| | | | VDNALQSGNSQESVTEQDSKDSTYSLSSTLTLSKADYE |
| | | | KHKVYACEVTHQGLSSPVTKSFNRGEC |
| | SEQ ID NO: 24 | VL DNA | GACGTGGTGATGACACAGAGCCCCCTGTCCCTGCCTG |
| | | | TGACCCCCGGGGAACCCGCTAGCATCAGCTGCAGAA |
| | | | GCAGCCAAAGCCTGGTGCACAGCAACAGAAACACCT |
| | | | ACCTGCACTGGTACCTGCAGAAGCCCGGGCAGAGCC |
| | | | CTCAGCTGCTGATCTACAAGGTGAGCAACAGATTCAG |
| | | | CGGCGTGCCCGACAGATTCTCCGGCAGCGGCAGCGG |
| | | | CACCGACTTCACCCTGAAGATCAGCAGAGTGGAGGC |
| | | | CGAGGACGTGGGCGTGTACTACTGCAGCCAAAACAC |
| | | | CCACGTGCCCCCCACCTTCGGCCAAGGCACCAAGCT |
| | | | GGAGATCAAGCGTACGGTGGCTGCACCATCTGTCTTC |
| | | | ATCTTCCCGCCATCTGATGAGCAGTTGAAATCTGGAA |
| | | | CTGCCTCTGTTGTGTGCCTGCTGAATAACTTCTATCCC |
| | | | AGAGAGGCCAAAGTACAGTGGAAGGTGGATAACGCC |
| | | | CTCCAATCGGGTAACTCCCAGGAGAGTGTCACAGAG |
| | | | CAGGACAGCAAGGACAGCACCTACAGCCTCAGCAGC |
| | | | ACCCTGACGCTGAGCAAAGCAGACTACGAGAAACAC |
| | | | AAAGTCTACGCCTGCGAAGTCACCCATCAGGGCCTGA |
| | | | GCTCGCCCGTCACAAAGAGCTTCAACAGGGGAGAGT |
| | | | GT |
| BE-774 | SEQ ID NO: 25 | VH AA | QVQLVQSGAEVKKPGASVKVSCKASGYTFTDYEMHW |
| | | | VRQAPGQGLEWMGALDPKTGDTAYSQKFKGRVTLTAD |
| | | | KSTSTAYMELSSLTSEDTAVYYCTRFYSYTYWGQGTLV |
| | | | TVSSASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPC |
| | | | PAPPAAGPSVFLFPPKPKDTLYITREPEVTCVVVDVSHE |
| | | | DPEVKFNWYVDGVEVHNAKTKPREEQYNSTYRVVSVL |
| | | | TVLHQDWLNGKEYKCKVSNKALAAPIEKTISKAKGQP |
| | | | REPQVYTLPPSREEMTKNQVSLTCLVKGFYPSDIAVEWE |
| | | | SNGQPENNYKTTPPVLDSDGSFFLYSKLTVDKSRWQQG |
| | | | NVFSCSVMHEALHNHYTQKSLSLSPGKGGGGSEVQLV |
| | | | ESGGGTVQPGDSLRLSCAASGRTFDNYAVGWFRQAPG |
| | | | KERQYVGTYWSGYAHYGDFENNRFTISRDNAKNTVYL |
| | | | QMDNLKPEDTAVYYCAISLKYPMATVTPDYQGQGTQV |
| | | | TVSS |
| | SEQ ID NO: 26 | VH DNA | CAGGTGCAGCTGGTGCAGTCTGGAGCAGAGGTGAAG |
| | | | AAGCCAGGAGCCAGCGTGAAGGTGTCCTGCAAGGCC |
| | | | TCTGGCTATACCTTCACAGACTACGAGATGCACTGGG |
| | | | TGCGGCAGGCACCAGGACAGGGACTGGAGTGGATGG |
| | | | GCGCCCTGGACCCTAAGACCGGCGATACAGCCTATAG |
| | | | CCAGAAGTTTAAGGGCAGAGTGACCCTGACAGCCGA |
| | | | CAAGTCCACCTCTACAGCCTACATGGAGCTGAGCTCC |
| | | | CTGACCAGCGAGGATACAGCCGTGTACTATTGTACCC |
| | | | GGTTCTACTCCTATACATACTGGGGCCAGGGCACCCT |
| | | | GGTGACAGTGTCTAGCGCTAGCACCAAGGGGCCCTC |
| | | | GGTCTTCCCCCTGGCACCCTCCTCCAAGAGTACTTCT |
| | | | GGGGGCACAGCGGCCCTGGGCTGCCTGGTCAAGGAC |
| | | | TACTTCCCCGAACCGGTGACGGTGTCGTGGAACTCAG |
| | | | GCGCCCTGACCAGCGGCGTGCACACCTTCCCGGCTGT |
| | | | CCTACAGTCCTCAGGACTCTACTCCCTCAGCAGCGTG |
| | | | GTGACCGTGCCCTCCAGCAGCTTGGGCACCCAGACC |
| | | | TACATCTGCAACGTGAATCACAAGCCCAGCAACACCA |
| | | | AGGTGGACAAGAAAGTTGAGCCAAAGTCCTGTGACA |
| | | | AGACCCACACATGCCCCCCTTGTCCTGCTCCACCAGC |
| | | | TGCAGGACCAAGCGTGTTCCTGTTTCCACCCAAGCCC |
| | | | AAGGATACCCTGtacATCaccCGGgagCCAGAGGTGACAT |
| | | | GCGTGGTGGTGGATGTGAGCCACGAGGACCCCGAGG |
| | | | TGAAGTTCAACTGGTATGTGGACGGCGTGGAGGTGC |
| | | | ACAATGCTAAGACCAAGCCCAGGGAGGAGCAGTACA |
| | | | ACTCCACCTATAGAGTGGTGTCTGTGCTGACAGTGCT |
| | | | GCACCAGGATTGGCTGAACGGCAAGGAGTATAAGTG |
| | | | CAAGGTGTCCAATAAGGCCCTGGCCGCTCCTATCGAG |
| | | | AAGACCATCTCTAAGGCCAAGGGCCAGCCCAGAGAG |
| | | | CCTCAGGTGTACACACTGCCTCCATCCCGGGAAGAGA |
| | | | TGACCAAGAACCAGGTGTCTCTGACATGTCTGGTCAA |
| | | | GGGCTTCTATCCCTCTGACATCGCCGTGGAGTGGGAG |
| | | | AGCAATGGCCAGCCTGAGAACAATTACAAGACCACA |
| | | | CCCCCTGTGCTGGATTCCGACGGCTCTTTCTTTCTGTA |
| | | | TAGCAAGCTGACCGTGGACAAGTCCCGGTGGCAGCA |
| | | | GGGCAACGTGTTCAGCTGTTCCGTGATGCACGAAGCT |
| | | | CTGCATAATCACTATACTCAGAAATCCCTGTCACTGTC |
| | | | ACCTGGTAAAGGTGGAGGCGGTTCAGAGGTGCAGCT |
| | | | CGTGGAGTCCGGGGGAGGAACGGTGCAGCCTGGGGA |
| | | | CTCTCTGAGACTCTCCTGTGCTGCCTCTGGACGCACC |
| | | | TTCGATAACTATGCCGTGGGCTGGTTCCGCCAGGCTC |
| | | | CAGGGAAGGAGCGTCAGTATGTCGGCACGTATTGGA |
| | | | GTGGTTACGCACACTATGGGGACTTTGAGAATAACCG |
| | | | ATTCACGATCTCCAGAGACAACGCCAAGAATACGGTG |
| | | | TATCTGCAAATGGACAACCTGAAACCTGAGGACACG |
| | | | GCCGTTTATTACTGTGCAATATCCCTTAAATATCCTATG |
| | | | GCGACAGTTACCCCAGACTACCAGGGCCAGGGGACC |
| | | | CAGGTCACCGTCTCCTCA |
| BE-933 | SEQ ID NO: 27 | VH AA | QVQLVQSGAEVKKPGASVKVSCKASGYTFTDYEMHW |
| | | | VRQAPGQGLEWMGALDPKTGDTAYSQKFKGRVTLTAD |
| | | | KSTSTAYMELSSLTSEDTAVYYCTRFYSYTYWGQGTLV |
| | | | TVSSASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPC |
| | | | PAPPAAGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALAAPIEKTISKAKGQP |
| | | | REPQVYTLPPSREEMTKNQVSLTCLVKGFYPSDIAVEWE |
| | | | SNGQPENNYKTTPPVLDSDGSFFLYSKLTVDKSRWQQG |
| | | | NVFSCSVMHEALHNHYTQKSLSLSPGKGGGGSEVQLV |
| | | | ESGGGTVQPGDSLRLSCAASGRTFDNYAVGWFRQAPG |
| | | | KERQYVGTYWSGYAHYGDFENNRFTISRDNAKNTVYL |
| | | | QMDNLKPEDTAVYYCAISLKYPMATVTPDYQGQGTQV |
| | | | TVSS |
| | SEQ ID NO: 28 | VH DNA | CAGGTGCAGCTGGTGCAGTCTGGAGCAGAGGTGAAG |
| | | | AAGCCAGGAGCCAGCGTGAAGGTGTCCTGCAAGGCC |
| | | | TCTGGCTATACCTTCACAGACTACGAGATGCACTGGG |
| | | | TGCGGCAGGCACCAGGACAGGGACTGGAGTGGATGG |
| | | | GCGCCCTGGACCCTAAGACCGGCGATACAGCCTATAG |
| | | | CCAGAAGTTTAAGGGCAGAGTGACCCTGACAGCCGA |
| | | | CAAGTCCACCTCTACAGCCTACATGGAGCTGAGCTCC |
| | | | CTGACCAGCGAGGATACAGCCGTGTACTATTGTACCC |
| | | | GGTTCTACTCCTATACATACTGGGGCCAGGGCACCCT |
| | | | GGTGACAGTGTCTAGCGCTAGCACCAAGGGGCCCTC |
| | | | GGTCTTCCCCCTGGCACCCTCCTCCAAGAGTACTTCT |
| | | | GGGGGCACAGCGGCCCTGGGCTGCCTGGTCAAGGAC |
| | | | TACTTCCCCGAACCGGTGACGGTGTCGTGGAACTCAG |
| | | | GCGCCCTGACCAGCGGCGTGCACACCTTCCCGGCTGT |
| | | | CCTACAGTCCTCAGGACTCTACTCCCTCAGCAGCGTG |
| | | | GTGACCGTGCCCTCCAGCAGCTTGGGCACCCAGACC |
| | | | TACATCTGCAACGTGAATCACAAGCCCAGCAACACCA |
| | | | AGGTGGACAAGAAAGTTGAGCCAAAGTCCTGTGACA |
| | | | AGACCCACACATGCCCCCCTTGTCCTGCTCCACCAGC |
| | | | TGCAGGACCAAGCGTGTTCCTGTTTCCACCCAAGCCC |
| | | | AAGGATACCCTGATGATCTCTCGGACCCCAGAGGTGA |
| | | | CATGCGTGGTGGTGGATGTGAGCCACGAGGACCCCG |
| | | | AGGTGAAGTTCAACTGGTATGTGGACGGCGTGGAGG |
| | | | TGCACAATGCTAAGACCAAGCCCAGGGAGGAGCAGT |
| | | | ACAACTCCACCTATAGAGTGGTGTCTGTGCTGACAGT |
| | | | GCTGCACCAGGATTGGCTGAACGGCAAGGAGTATAA |
| | | | GTGCAAGGTGTCCAATAAGGCCCTGGCCGCTCCTATC |
| | | | GAGAAGACCATCTCTAAGGCCAAGGGCCAGCCCAGA |
| | | | GAGCCTCAGGTGTACACACTGCCTCCATCCCGGGAAG |
| | | | AGATGACCAAGAACCAGGTGTCTCTGACATGTCTGGT |
| | | | CAAGGGCTTCTATCCCTCTGACATCGCCGTGGAGTGG |
| | | | GAGAGCAATGGCCAGCCTGAGAACAATTACAAGACC |
| | | | ACACCCCCTGTGCTGGATTCCGACGGCTCTTTCTTTCT |
| | | | GTATAGCAAGCTGACCGTGGACAAGTCCCGGTGGCA |
| | | | GCAGGGCAACGTGTTCAGCTGTTCCGTGATGCACGA |
| | | | AGCTCTGCATAATCACTATACTCAGAAATCCCTGTCAC |
| | | | TGTCACCTGGTAAAGGTGGAGGCGGTTCAGAGGTGC |
| | | | AGCTCGTGGAGTCCGGGGGAGGAACGGTGCAGCCTG |
| | | | GGGACTCTCTGAGACTCTCCTGTGCTGCCTCTGGACG |
| | | | CACCTTCGATAACTATGCCGTGGGCTGGTTCCGCCAG |
| | | | GCTCCAGGGAAGGAGCGTCAGTATGTCGGCACGTATT |
| | | | GGAGTGGTTACGCACACTATGGGGACTTTGAGAATAA |
| | | | CCGATTCACGATCTCCAGAGACAACGCCAAGAATACG |
| | | | GTGTATCTGCAAATGGACAACCTGAAACCTGAGGACA |
| | | | CGGCCGTTTATTACTGTGCAATATCCCTTAAATATCCTA |
| | | | TGGCGACAGTTACCCCAGACTACCAGGGCCAGGGGA |
| | | | CCCAGGTCACCGTCTCCTCA |
| BE-653 | SEQ ID NO: 29 | VH AA | QVQLVQSGAEVKKPGASVKVSCKASGYTFTDYEMHW |
| | | | VRQAPGQGLEWMGALDPKTGDTAYSQKFKGRVTLTAD |
| | | | KSTSTAYMELSSLTSEDTAVYYCTRFYSYTYWGQGTLV |
| | | | TVSSASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPC |
| | | | PAPPAAGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALAAPIEKTISKAKGQP |
| | | | REPQVYTLPPSREEMTKNQVSLTCLVKGFYPSDIAVEWE |
| | | | SNGQPENNYKTTPPVLDSDGSFFLYSKLTVDKSRWQQG |
| | | | NVFSCSVMHEALHNHYTQKSLSLSPGKGGGGSEVQLV |
| | | | ESGGGLVQPGGSLRLSCAASGRTFDNYAVGWFRQAPGK |
| | | | EREYVGTYWSGYAHYGDFENNRFTISRDNSKNTVYLQ |
| | | | MNNLRPEDTAVYYCAISLKYPMATVTPDYQGQGTQVT |
| | | | VSS |
| | SEQ ID NO: 30 | VH DNA | CAAGTGCAGCTGGTGCAGAGCGGCGCCGAGGTGAAG |
| | | | AAGCCCGGCGCTAGCGTGAAGGTGAGCTGCAAGGCT |
| | | | AGCGGCTACACCTTCACCGACTACGAAATGCACTGGG |
| | | | TCAGACAAGCCCCCGGCCAAGGCCTGGAATGGATGG |
| | | | GCGCCCTGGACCCCAAGACCGGCGACACCGCCTACA |
| | | | GCCAAAAGTTCAAGGGCAGAGTGACCCTGACCGCCG |
| | | | ACAAGAGCACAAGCACCGCCTACATGGAGCTGAGCA |
| | | | GCCTGACAAGCGAGGACACCGCCGTCTACTACTGCA |
| | | | CAAGATTCTACAGCTACACCTACTGGGGCCAAGGCAC |
| | | | CCTCGTGACCGTGAGCTCCGCTAGCACAAAGGGCCCT |
| | | | AGCGTGTTCCCCCTCGCTCCTAGCAGCAAGAGCACCT |
| | | | CCGGGGGCACAGCCGCCCTGGGCTGCCTGGTGAAGG |
| | | | ACTACTTCCCCGAGCCCGTGACCGTGAGCTGGAACA |
| | | | GCGGCGCCCTCACAAGCGGCGTGCATACCTTCCCCGC |
| | | | CGTGCTGCAGAGCAGCGGCCTGTACAGCCTGAGCAG |
| | | | CGTGGTGACCGTGCCTAGCAGCAGCCTGGGCACACA |
| | | | GACCTACATCTGCAACGTGAACCACAAGCCTAGCAAC |
| | | | ACCAAGGTGGACAAGAAGGTGGAGCCCAAGTCCTGT |
| | | | GATAAGACCCACACATGCCCTCCCTGCCCCGCCCCTC |
| | | | CCGCCGCCGGCCCTTCCGTGTTCCTGTTCCCCCCCAA |
| | | | GCCCAAGGACACCCTGATGATCAGCAGAACCCCCGA |
| | | | GGTGACCTGCGTGGTCGTGGACGTGAGCCACGAGGA |
| | | | CCCCGAGGTGAAGTTCAACTGGTACGTGGACGGCGT |
| | | | GGAGGTGCACAACGCCAAGACCAAGCCTAGAGAGGA |
| | | | GCAGTACAACAGCACCTACAGAGTGGTGAGCGTGCT |
| | | | GACCGTGCTGCACCAAGACTGGCTGAACGGCAAGGA |
| | | | GTACAAGTGCAAGGTGAGCAACAAGGCCCTGGCCGC |
| | | | CCCCATCGAGAAGACCATCAGCAAGGCCAAGGGGCA |
| | | | GCCTAGAGAGCCCCAAGTGTACACCCTGCCCCCTAGC |
| | | | AGAGAGGAGATGACCAAGAACCAAGTGAGCCTGACC |
| | | | TGCCTCGTGAAGGGCTTCTACCCTAGCGACATCGCCG |
| | | | TGGAGTGGGAGAGCAACGGGCAGCCCGAGAACAAC |
| | | | TACAAGACCACCCCCCCCGTGCTGGACAGCGACGGC |
| | | | AGCTTCTTCCTGTACAGCAAGCTGACCGTGGACAAGA |
| | | | GCAGATGGCAGCAAGGCAACGTGTTCAGCTGCAGCG |
| | | | TGATGCACGAGGCCCTGCACAACCACTACACCCAAA |
| | | | AAAGCCTCAGCCTCAGCCCCGGCAAGGGCGGGGGCG |
| | | | GCTCCGAAGTGCAGCTGGTGGAGTCCGGGGGCGGCC |
| | | | TGGTCCAACCCGGGGGCAGCCTGAGACTGAGCTGCG |
| | | | CCGCTAGCGGCAGAACCTTCGACAACTACGCCGTGG |
| | | | GCTGGTTCAGACAAGCCCCCGGCAAGGAGAGAGAGT |
| | | | ACGTGGGCACCTACTGGAGCGGCTACGCCCACTACG |
| | | | GCGACTTCGAGAACAACAGATTCACCATCAGCAGAG |
| | | | ACAACAGCAAGAACACCGTGTACCTGCAGATGAACA |
| | | | ACCTGAGACCCGAGGACACCGCCGTGTACTACTGCG |
| | | | CCATCAGCCTGAAGTACCCCATGGCCACCGTGACCCC |
| | | | CGACTACCAAGGCCAAGGCACCCAAGTGACCGTGAG |
| | | | CAGCTGA |
| BE-647 | SEQ ID NO: 31 | VH AA | QVQLVQSGAEVKKPGASVKVSCKASGYTFTDYEMHW |
| | | | VRQAPGQGLEWMGALDPKTGDTAYSQKFKGRVTLTAD |
| | | | KSTSTAYMELSSLTSEDTAVYYCTRFYSYTYWGQGTLV |
| | | | TVSSASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPC |
| | | | PAPPAAGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALAAPIEKTISKAKGQP |
| | | | REPQVYTLPPSREEMTKNQVSLTCLVKGFYPSDIAVEWE |
| | | | SNGQPENNYKTTPPVLDSDGSFFLYSKLTVDKSRWQQG |
| | | | NVFSCSVMHEALHNHYTQKSLSLSPGKGGGGSEVQLV |
| | | | ESGGGLVQPGGSLRLSCAASGRTFDNYAVGWFRQAPGK |
| | | | EREYVGTYWSGYAHYGDFEKGRFTISRDNAKNTVYLQ |
| | | | MNNLRPEDTAVYYCAISLKYPMATVTPDYQGQGTQVT |
| | | | VSS |
| | SEQ ID NO: 32 | VH DNA | CAAGTGCAGCTGGTGCAGAGCGGCGCCGAGGTGAAG |
| | | | AAGCCCGGCGCTAGCGTGAAGGTGAGCTGCAAGGCT |
| | | | AGCGGCTACACCTTCACCGACTACGAAATGCACTGGG |
| | | | TCAGACAAGCCCCCGGCCAAGGCCTGGAATGGATGG |
| | | | GCGCCCTGGACCCCAAGACCGGCGACACCGCCTACA |
| | | | GCCAAAAGTTCAAGGGCAGAGTGACCCTGACCGCCG |
| | | | ACAAGAGCACAAGCACCGCCTACATGGAGCTGAGCA |
| | | | GCCTGACAAGCGAGGACACCGCCGTCTACTACTGCA |
| | | | CAAGATTCTACAGCTACACCTACTGGGGCCAAGGCAC |
| | | | CCTCGTGACCGTGAGCTCCGCTAGCACAAAGGGCCCT |
| | | | AGCGTGTTCCCCCTCGCTCCTAGCAGCAAGAGCACCT |
| | | | CCGGGGGCACAGCCGCCCTGGGCTGCCTGGTGAAGG |
| | | | ACTACTTCCCCGAGCCCGTGACCGTGAGCTGGAACA |
| | | | GCGGCGCCCTCACAAGCGGCGTGCATACCTTCCCCGC |
| | | | CGTGCTGCAGAGCAGCGGCCTGTACAGCCTGAGCAG |
| | | | CGTGGTGACCGTGCCTAGCAGCAGCCTGGGCACACA |
| | | | GACCTACATCTGCAACGTGAACCACAAGCCTAGCAAC |
| | | | ACCAAGGTGGACAAGAAGGTGGAGCCCAAGTCCTGT |
| | | | GATAAGACCCACACATGCCCTCCCTGCCCCGCCCCTC |
| | | | CCGCCGCCGGCCCTTCCGTGTTCCTGTTCCCCCCCAA |
| | | | GCCCAAGGACACCCTGATGATCAGCAGAACCCCCGA |
| | | | GGTGACCTGCGTGGTCGTGGACGTGAGCCACGAGGA |
| | | | CCCCGAGGTGAAGTTCAACTGGTACGTGGACGGCGT |
| | | | GGAGGTGCACAACGCCAAGACCAAGCCTAGAGAGGA |
| | | | GCAGTACAACAGCACCTACAGAGTGGTGAGCGTGCT |
| | | | GACCGTGCTGCACCAAGACTGGCTGAACGGCAAGGA |
| | | | GTACAAGTGCAAGGTGAGCAACAAGGCCCTGGCCGC |
| | | | CCCCATCGAGAAGACCATCAGCAAGGCCAAGGGGCA |
| | | | GCCTAGAGAGCCCCAAGTGTACACCCTGCCCCCTAGC |
| | | | AGAGAGGAGATGACCAAGAACCAAGTGAGCCTGACC |
| | | | TGCCTCGTGAAGGGCTTCTACCCTAGCGACATCGCCG |
| | | | TGGAGTGGGAGAGCAACGGGCAGCCCGAGAACAAC |
| | | | TACAAGACCACCCCCCCCGTGCTGGACAGCGACGGC |
| | | | AGCTTCTTCCTGTACAGCAAGCTGACCGTGGACAAGA |
| | | | GCAGATGGCAGCAAGGCAACGTGTTCAGCTGCAGCG |
| | | | TGATGCACGAGGCCCTGCACAACCACTACACCCAAA |
| | | | AAAGCCTCAGCCTCAGCCCCGGCAAGGGCGGGGGCG |
| | | | GCTCCGAAGTGCAGCTGGTGGAGTCCGGGGGCGGCC |
| | | | TGGTCCAACCCGGGGGCAGCCTGAGACTGAGCTGCG |
| | | | CCGCTAGCGGCAGAACCTTCGACAACTACGCCGTGG |
| | | | GCTGGTTCAGACAAGCCCCCGGCAAGGAGAGAGAGT |
| | | | ACGTGGGCACCTACTGGAGCGGCTACGCCCACTACG |
| | | | GCGACTTCGAGAAGGGGAGATTCACCATCAGCAGAG |
| | | | ACAACGCCAAGAACACCGTGTACCTGCAGATGAACA |
| | | | ACCTGAGACCCGAGGACACCGCCGTGTACTACTGCG |
| | | | CCATCAGCCTGAAGTACCCCATGGCCACCGTGACCCC |
| | | | CGACTACCAAGGCCAAGGCACCCAAGTGACCGTGAG |
| | | | CAGCTGA |
| | SEQ ID NO: 33 | VH AA | QVQLVQSGAEVKKPGASVKVSCKASGYTFTDYEMHW |
| | | | VRQAPGQGLEWMGALDPKTGDTAYSQKFKGRVTLTAD |
| | | | KSTSTAYMELSSLTSEDTAVYYCTRFYSYTYWGQGTLV |
| | | | TVSSASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPC |
| | | | PAPPAAGPSVFLFPPKPKDTLYITREPEVTCVVVDVSHE |
| | | | DPEVKFNWYVDGVEVHNAKTKPREEQYNSTYRVVSVL |
| | | | TVLHQDWLNGKEYKCKVSNKALAAPIEKTISKAKGQP |
| | | | REPQVYTLPPSREEMTKNQVSLTCLVKGFYPSDIAVEWE |
| | | | SNGQPENNYKTTPPVLDSDGSFFLYSKLTVDKSRWQQG |
| | | | NVFSCSVMHEALHNHYTQKSLSLSPGKGGGGSEVQLV |
| | | | ESGGGLVQPGGSLRLSCAASGRTFDNYAVGWFRQAPGK |
| | | | EREYVGTYWSGYAHYGDFEKGRFTISRDNAKNTVYLQ |
| | | | MNNLRPEDTAVYYCAISLKYPMATVTPDYQGQGTQVT |
| | | | VSS |
| BE-621 | SEQ ID NO: 34 | VH DNA | CAAGTGCAGCTGGTGCAGAGCGGCGCCGAGGTGAAG |
| | | | AAGCCCGGCGCTAGCGTGAAGGTGAGCTGCAAGGCT |
| | | | AGCGGCTACACCTTCACCGACTACGAAATGCACTGGG |
| | | | TCAGACAAGCCCCCGGCCAAGGCCTGGAATGGATGG |
| | | | GCGCCCTGGACCCCAAGACCGGCGACACCGCCTACA |
| | | | GCCAAAAGTTCAAGGGCAGAGTGACCCTGACCGCCG |
| | | | ACAAGAGCACAAGCACCGCCTACATGGAGCTGAGCA |
| | | | GCCTGACAAGCGAGGACACCGCCGTCTACTACTGCA |
| | | | CAAGATTCTACAGCTACACCTACTGGGGCCAAGGCAC |
| | | | CCTCGTGACCGTGAGCTCCGCTAGCACAAAGGGCCCT |
| | | | AGCGTGTTCCCCCTCGCTCCTAGCAGCAAGAGCACCT |
| | | | CCGGGGGCACAGCCGCCCTGGGCTGCCTGGTGAAGG |
| | | | ACTACTTCCCCGAGCCCGTGACCGTGAGCTGGAACA |
| | | | GCGGCGCCCTCACAAGCGGCGTGCATACCTTCCCCGC |
| | | | CGTGCTGCAGAGCAGCGGCCTGTACAGCCTGAGCAG |
| | | | CGTGGTGACCGTGCCTAGCAGCAGCCTGGGCACACA |
| | | | GACCTACATCTGCAACGTGAACCACAAGCCTAGCAAC |
| | | | ACCAAGGTGGACAAGAAGGTGGAGCCCAAGTCCTGT |
| | | | GATAAGACCCACACATGCCCTCCCTGCCCCGCCCCTC |
| | | | CCGCCGCCGGCCCTTCCGTGTTCCTGTTCCCCCCCAA |
| | | | GCCCAAGGACACCCTGTACATCACCAGAGAGCCCGA |
| | | | GGTGACCTGCGTGGTCGTGGACGTGAGCCACGAGGA |
| | | | CCCCGAGGTGAAGTTCAACTGGTACGTGGACGGCGT |
| | | | GGAGGTGCACAACGCCAAGACCAAGCCTAGAGAGGA |
| | | | GCAGTACAACAGCACCTACAGAGTGGTGAGCGTGCT |
| | | | GACCGTGCTGCACCAAGACTGGCTGAACGGCAAGGA |
| | | | GTACAAGTGCAAGGTGAGCAACAAGGCCCTGGCCGC |
| | | | CCCCATCGAGAAGACCATCAGCAAGGCCAAGGGGCA |
| | | | GCCTAGAGAGCCCCAAGTGTACACCCTGCCCCCTAGC |
| | | | AGAGAGGAGATGACCAAGAACCAAGTGAGCCTGACC |
| | | | TGCCTCGTGAAGGGCTTCTACCCTAGCGACATCGCCG |
| | | | TGGAGTGGGAGAGCAACGGGCAGCCCGAGAACAAC |
| | | | TACAAGACCACCCCCCCCGTGCTGGACAGCGACGGC |
| | | | AGCTTCTTCCTGTACAGCAAGCTGACCGTGGACAAGA |
| | | | GCAGATGGCAGCAAGGCAACGTGTTCAGCTGCAGCG |
| | | | TGATGCACGAGGCCCTGCACAACCACTACACCCAAA |
| | | | AAAGCCTCAGCCTCAGCCCCGGCAAGGGCGGGGGCG |
| | | | GCTCCGAAGTGCAGCTGGTGGAGTCCGGGGGCGGCC |
| | | | TGGTCCAACCCGGGGGCAGCCTGAGACTGAGCTGCG |
| | | | CCGCTAGCGGCAGAACCTTCGACAACTACGCCGTGG |
| | | | GCTGGTTCAGACAAGCCCCCGGCAAGGAGAGAGAGT |
| | | | ACGTGGGCACCTACTGGAGCGGCTACGCCCACTACG |
| | | | GCGACTTCGAGAAGGGGAGATTCACCATCAGCAGAG |
| | | | ACAACGCCAAGAACACCGTGTACCTGCAGATGAACA |
| | | | ACCTGAGACCCGAGGACACCGCCGTGTACTACTGCG |
| | | | CCATCAGCCTGAAGTACCCCATGGCCACCGTGACCCC |
| | | | CGACTACCAAGGCCAAGGCACCCAAGTGACCGTGAG |
| | | | CAGC |
| Human | SEQ ID NO: 35 | AA | MGNSCYNIVATLLLVLNFERTRSLQDPCSNCPAGTFCDN |
| CD137-full | | | NRNQICSPCPPNSFSSAGGQRTCDICRQCKGVFRTRKEC |
| length | | | SSTSNAECDCTPGFHCLGAGCSMCEQDCKQGQELTKK |
| | | | GCKDCCFGTFNDQKRGICRPWTNCSLDGKSVLVNGTK |
| | | | ERDVVCGPSPADLSPGASSVTPPAPAREPGHSPQIISFFLA |
| | | | LTSTALLFLLFFLTLRFSVVKRGRKKLLYIFKQPFMRPVQ |
| | | | TTQEEDGCSCRFPEEEEGGCEL |
| | SEQ ID NO: 36 | DNA | ATGGGAAACAGCTGTTACAACATAGTAGCCACTCTGT |
| | | | TGCTGGTCCTCAACTTTGAGAGGACAAGATCATTGCA |
| | | | GGATCCTTGTAGTAACTGCCCAGCTGGTACATTCTGTG |
| | | | ATAATAACAGGAATCAGATTTGCAGTCCCTGTCCTCCA |
| | | | AATAGTTTCTCCAGCGCAGGTGGACAAAGGACCTGTG |
| | | | ACATATGCAGGCAGTGTAAAGGTGTTTTCAGGACCAG |
| | | | GAAGGAGTGTTCCTCCACCAGCAATGCAGAGTGTGA |
| | | | CTGCACTCCAGGGTTTCACTGCCTGGGGGCAGGATGC |
| | | | AGCATGTGTGAACAGGATTGTAAACAAGGTCAAGAA |
| | | | CTGACAAAAAAAGGTTGTAAAGACTGTTGCTTTGGG |
| | | | ACATTTAACGATCAGAAACGTGGCATCTGTCGACCCT |
| | | | GGACAAACTGTTCTTTGGATGGAAAGTCTGTGCTTGT |
| | | | GAATGGGACGAAGGAGAGGGACGTGGTCTGTGGACC |
| | | | ATCTCCAGCCGACCTCTCTCCGGGAGCATCCTCTGTG |
| | | | ACCCCGCCTGCCCCTGCGAGAGAGCCAGGACACTCT |
| | | | CCGCAGATCATCTCCTTCTTTCTTGCGCTGACGTCGAC |
| | | | TGCGTTGCTCTTCCTGCTGTTCTTCCTCACGCTCCGTT |
| | | | TCTCTGTTGTTAAACGGGGCAGAAAGAAACTCCTGTA |
| | | | TATATTCAAACAACCATTTATGAGACCAGTACAAACTA |
| | | | CTCAAGAGGAAGATGGCTGTAGCTGCCGATTTCCAGA |
| | | | AGAAGAAGAAGGAGGATGTGAACTG |
| Human | SEQ ID NO: 37 | AA | MEYASDASLDPEAPWPPAPRARACRVLPWALVAGLLLL |
| CD137- | | | LLLAAACAVFLACPWAVSGARASPGSAASPRLREGPELS |
| ligand full | | | PDDPAGLLDLRQGMFAQLVAQNVLLIDGPLSWYSDPGL |
| length | | | AGVSLTGGLSYKEDTKELVVAKAGVYYVFFQLELRRVV |
| | | | AGEGSGSVSLALHLQPLRSAAGAAALALTVDLPPASSE |
| | | | ARNSAFGFQGRLLHLSAGQRLGVHLHTEARARHAWQL |
| | | | TQGATVLGLFRVTPEIPAGLPSPRSE |
| | SEQ ID NO: 38 | DNA | ATGGAATACGCCTCTGACGCTTCACTGGACCCCGAAG |
| | | | CCCCGTGGCCTCCCGCGCCCCGCGCTCGCGCCTGCCG |
| | | | CGTACTGCCTTGGGCCCTGGTCGCGGGGCTGCTGCTG |
| | | | CTGCTGCTGCTCGCTGCCGCCTGCGCCGTCTTCCTCG |
| | | | CCTGCCCCTGGGCCGTGTCCGGGGCTCGCGCCTCGCC |
| | | | CGGCTCCGCGGCCAGCCCGAGACTCCGCGAGGGTCC |
| | | | CGAGCTTTCGCCCGACGATCCCGCCGGCCTCTTGGAC |
| | | | CTGCGGCAGGGCATGTTTGCGCAGCTGGTGGCCCAA |
| | | | AATGTTCTGCTGATCGATGGGCCCCTGAGCTGGTACA |
| | | | GTGACCCAGGCCTGGCAGGCGTGTCCCTGACGGGGG |
| | | | GCCTGAGCTACAAAGAGGACACGAAGGAGCTGGTGG |
| | | | TGGCCAAGGCTGGAGTCTACTATGTCTTCTTTCAACTA |
| | | | GAGCTGCGGCGCGTGGTGGCCGGCGAGGGCTCAGGC |
| | | | TCCGTTTCACTTGCGCTGCACCTGCAGCCACTGCGCT |
| | | | CTGCTGCTGGGGCCGCCGCCCTGGCTTTGACCGTGGA |
| | | | CCTGCCACCCGCCTCCTCCGAGGCTCGGAACTCGGCC |
| | | | TTCGGTTTCCAGGGCCGCTTGCTGCACCTGAGTGCCG |
| | | | GCCAGCGCCTGGGCGTCCATCTTCACACTGAGGCCAG |
| | | | GGCACGCCATGCCTGGCAGCTTACCCAGGGCGCCAC |
| | | | AGTCTTGGGACTCTTCCGGGTGACCCCCGAAATCCCA |
| | | | GCCGGACTCCCTTCACCGAGGTCGGAATAA |
| mIgG2a | SEQ ID NO: 39 | AA | EPRGPTIKPCPPCKCPAPNLLGGPSVFIFPPKIKDVLMISL |
| | | | SPIVTCVVVDVSEDDPDVQISWFVNNVEVHTAQTQTHR |
| | | | EDYNSTLRVVSALPIQHQDWMSGKEFKCKVNNKDLPA |
| | | | PIERTISKPKGSVRAPQVYVLPPPEEEMTKKQVTLTCMV |
| | | | TDFMPEDIYVEWTNNGKTELNYKNTEPVLDSDGSYFM |
| | | | YSKLRVEKKNWVERNSYSCSVVHEGLHNHHTTKSFSR |
| | | | TPGK |
| | SEQ ID NO: 40 | DNA | GAGCCCAGAGGGCCCACAATCAAGCCCTGTCCTCCAT |
| | | | GCAAATGCCCAGCACCTAACCTCTTGGGTGGACCATC |
| | | | CGTCTTCATCTTCCCTCCAAAGATCAAGGATGTACTCA |
| | | | TGATCTCCCTGAGCCCCATAGTCACATGTGTGGTGGT |
| | | | GGATGTGAGCGAGGATGACCCAGATGTCCAGATCAGC |
| | | | TGGTTTGTGAACAACGTGGAAGTACACACAGCTCAG |
| | | | ACACAAACCCATAGAGAGGATTACAACAGTACTCTCC |
| | | | GGGTGGTCAGTGCCCTCCCCATCCAGCACCAGGACTG |
| | | | GATGAGTGGCAAGGAGTTCAAATGCAAGGTCAACAA |
| | | | CAAAGACCTCCCAGCGCCCATCGAGAGAACCATCTC |
| | | | AAAACCCAAAGGGTCAGTAAGAGCTCCACAGGTATAT |
| | | | GTCTTGCCTCCACCAGAAGAAGAGATGACTAAGAAA |
| | | | CAGGTCACTCTGACCTGCATGGTCACAGACTTCATGC |
| | | | CTGAAGACATTTACGTGGAGTGGACCAACAACGGGA |
| | | | AAACAGAGCTAAACTACAAGAACACTGAACCAGTCC |
| | | | TGGACTCTGATGGTTCTTACTTCATGTACAGCAAGCTG |
| | | | AGAGTGGAAAAGAAGAACTGGGTGGAAAGAAATAG |
| | | | CTACTCCTGTTCAGTGGTCCACGAGGGTCTGCACAAT |
| | | | CACCACACGACTAAGAGCTTCTCCCGGACTCCGGGTA |
| | | | AA |
| Cyno CD137 | SEQ ID NO: | AA | LQDLCSNCPAGTFCDNNRSQICSPCPPNSFSSAGGQRTC |
| ECD | 102 | | DICRQCKGVFKTRKECSSTSNAECDCISGYHCLGAECS |
| | | | MCEQDCKQGQELTKKGCKDCCFGTFNDQKRGICRPWT |
| | | | NCSLDGKSVLVNGTKERDVVCGPSPADLSPGASSATPPA |
| | | | PAREPGHSPQEPRGPTIKPCPPCKCPAPNLLGGPSVFIFPP |
| | | | KIKDVLMISLSPIVTCVVVDVSEDDPDVQISWFVNNVE |
| | | | VHTAQTQTHREDYNSTLRVVSALPIQHQDWMSGKEFK |
| | | | CKVNNKDLPAPIERTISKPKGSVRAPQVYVLPPPEEEMT |
| | | | KKQVTLTCMVTDFMPEDIYVEWTNNGKTELNYKNTEP |
| | | | VLDSDGSYFMYSKLRVEKKNWVERNSYSCSVVHEGLH |
| | | | NHHTTKSFSRTPGK |
| Tislelizumab | SEQ ID NO: | AA | QVQLQESGPGLVKPSETLSLTCTVSGFSLTSYGVHWIRQ |
| VH | 103 | | PPGKGLEWIGVIYADGSTNYNPSLKSRVTISKDTSKNQV |
| | | | SLKLSSVTAADTAVYYCARAYGNYWYIDVWGQGTTVT |
| | | | VSS |
| Tislelizumab | SEQ ID NO: | AA | DIVMTQSPDSLAVSLGERATINCKSSESVSNDVAWYQQ |
| VL | 104 | | KPGQPPKLLINYAFHRFTGVPDRFSGSGYGTDFTLTISSL |
| | | | QAEDVAVYYCHQAYSSPYTFGQGTKLEIK |

### Example 21. Structural and Functional CD137 Epitope Mapping

To better understand how the anti-CD137 single domain antibody arm is capable of high affinity for CD137, and robust agonist of CD137/CD137L interaction, the crystal structure of VHH (BGA-2524) in complex with CD137 was determined.

### A. CD137 and VHH (BGA-2524) Expression, Purification, and Crystallization

Human CD137 ectodomain containing partial three CRDs (CRD1-3; amino acids 24-105 of SEQ ID NO: 35 (human CD137-full length)) was expressed in HEK293G cells. The cDNA coding CD137 was cloned into pMAX vector with an N-terminal secretion sequence and a C-terminal TEV cleavage site followed by an Fc tag. The culture supernatant containing the secreted CD137-Fc fusion protein was mixed with Mab Select Sure™ resin (GE Healthcare Life Sciences) for 3 hours at 4° C. The protein was washed with buffer containing 20 mM Tris-HCl pH 8.0, 150 mM NaCl, then eluted with 50 mM acetic acid (adjust pH value to 3.5 with 5 M NaOH), and finally neutralized with 1/10 CV 1.0 M Tris-HCl pH 8.0. The eluted protein was mixed with TEV proteases (10:1 molar ratio) and dialyzed against buffer (20 mM Tris-HCl, pH 8.0, 100 mM NaCl) at 4° C. overnight. The mixture was loaded onto a Ni-NTA column (Qiagen) and Mab Select Sure™ resin to remove the TEV proteases and Fc tag, and then the flow-through was further purified by size-exclusion chromatography in buffer (20 mM Tris pH 8.0, 100 mM NaCl) using a HiLoad 16/600 Superdex™ 75 μg column (GE Healthcare Life Sciences).

DNA sequence encoding VHH (BGA-2524) was cloned into a PET21a vector with N-terminal HIS-MBP tag followed by TEV protease site. Protein expression in Shuffle T7 was induced at OD600 of 0.6-1.0 with 1 mM IPTG at 18° C. for 16 h. The cells were harvested by centrifugation at 7,000 g, 10 min. The cell pellets were re-suspended in lysis buffer (50 mM Tris-HCl pH 8.0, 300 mM NaCl) and lysed under sonication on ice. The lysate then was centrifuged at 48,000 g at 4° C. for 30 min. The supernatant was mixed with Talon resin and batched at 4° C. for 3 hours. The resin was washed with lysis buffer containing 5 mM imidazole, the protein was eluted in lysis buffer with additional 100 mM imidazole. The eluate was mixed with TEV proteases (10:1 molar ratio) and dialyzed against buffer (20 mM Tris-HCl, pH 8.0, 100 mM NaCl) at 4° C. overnight. The mixture was loaded onto a Talon column to remove the TEV proteases and HIS-MBP tag, and then the flow-through was further purified by size-exclusion chromatography in buffer (20 mM Tris pH 8.0, 100 mM NaCl) using a HiLoad 16/600 Superdex™ 75 pg column (GE Healthcare Life Sciences).

Excess of purified CD137 was mixed with purified VHH (BGA-2524) (1.2:1 molar ratio) to generate the CD137/VHH (BGA-2524) complex. The complex was then further purified by gel filtration in buffer (20 mM Tris pH 8.0, 100 mM NaCl) using a Superdex™ 75 Increase 10/300 column (GE Healthcare Life Sciences). The CD137/VHH (BGA-2524) complex (10 mg/ml) was crystallized in 18% PEG 4000, 0.1 M Tris pH 8.7, 0.2 M Li₂SO₄. Crystals cryoprotected with 20% PEG 4000, 0.1 M Tris pH 8.7, 0.2 M Li₂SO₄, 10% Glycerol were flash frozen in liquid nitrogen. The X-ray diffraction data was collected at beamline BL02U1 at Shanghai synchrotron radiation facility (Shanghai, China).

### B. Data Collection and Structure Solution

The X-ray diffraction data was collected under cryo cooled conditions at 100 Kelvin at beamline BL02U1 in Shanghai synchrotron radiation facility (Shanghai, China). Diffraction images were processed with the integrated data processing software employing XDS (Kabsch, W., Xds. Acta Crystallogr D Biol Crystallogr, 2010. 66(Pt 2): p. 125-32). The structure of human CD137 (PDB: 6MGP) and in-house VHH model were used as search models. The initial solution was found with molecular replacement program PHASER (McCoy, A. J., et al., Phaser crystallographic software. J Appl Crystallogr, 2007. 40(Pt 4): p. 658-674). Then this model was iterative manually built with program COOT (Emsley, P. and K. Cowtan, Coot: model-building tools for molecular graphics. Acta Crystallogr D Biol Crystallogr, 2004. 60(Pt 12 Pt 1): p. 2126-32) and refinement using PHENIX (Adams, P. D., et al., PHENIX: a comprehensive Python-based system for macromolecular structure solution. Acta Crystallogr D Biol Crystallogr, 2010. 66(Pt 2): p. 213-21). The final model was refined to acceptable R and R free values and Ramachandran statistics (calculated by Molprobity). Data processing and refinement statistics can be found in Table 18.

TABLE 18
Data collection and refinement statistics

| Crystals | VHH (BGA-2524)/CD137 complex | |
|---|---|---|
| Data collection | | |
| X-ray source | BL02U1 | |
| Wavelength (Å) | 0.9792 | |
| Space group | P2₁ | |
| Cell Dimensions | | |
| a (Å) | 41.6755 | |
| b (Å) | 86.6772 | |
| c (Å) | 58.1101 | |
| α (°) | 90 | |
| ß (°) | 104.0174 | |
| Υ (°) | 90 | |
| Resolution (Å) | 86.68-1.71 | (1.75-1.71) |
| Rsym (%) | 4.4 | (96.7) |
| <I/σ> | 9.9 | (1.5) |
| CC_{1/2} | 99.6 | (40.9) |
| Redundancy | 6.5 | (5.5) |
| Completeness (%) | 100 | (100) |

| | |
|---|---|
| Refinement | |
| Resolution (Å) | 29.63-1.71 |
| No. reflections | 43253 |
| Rwork/Rfree ^{b}(%) | 21.86/24.98 |
| r.m.s.d. ^{c}bonds/angles | 0.009/1.678 |
| Number of atoms | |
| Protein | 2870 |
| Water | 301 |
| B factors | |
| Protein | 31.13 |
| Water | 36.41 |
| Ramachandran | |
| Favored (%) | 98.64 |
| Allowed (%) | 1.36 |
| Outlier (%) | 0 |

^{a} Values in parentheses are those of the highest resolution shell.
^{b}Calculated from about 5% of the reflection set aside during refinement
^{c}r.m.s.d., root mean square deviation

### C. The Structure of VHH (BGA-2524) Bound to Human CD137 Dimer

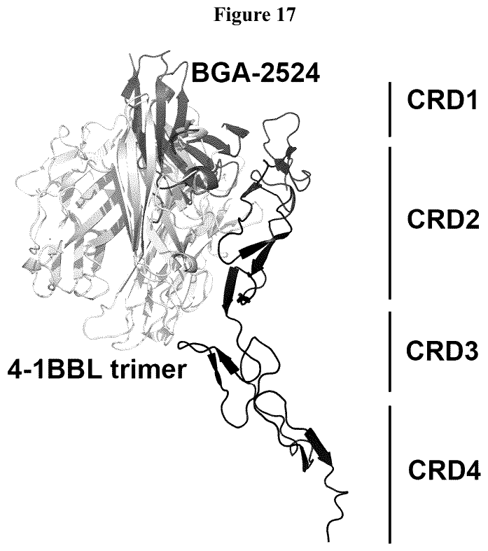

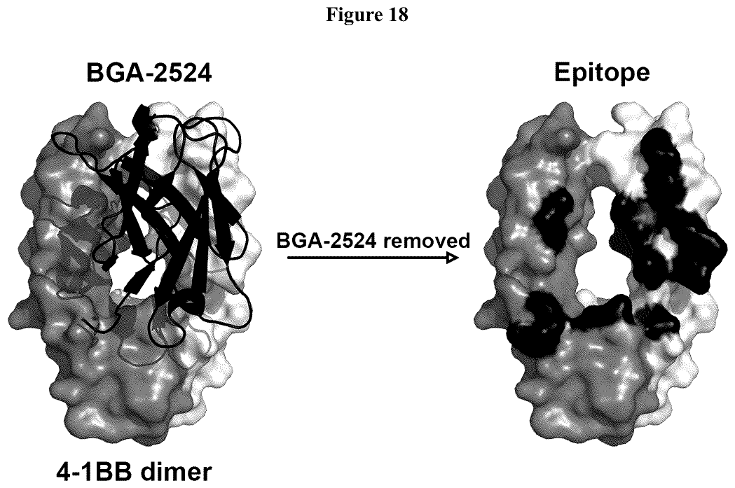

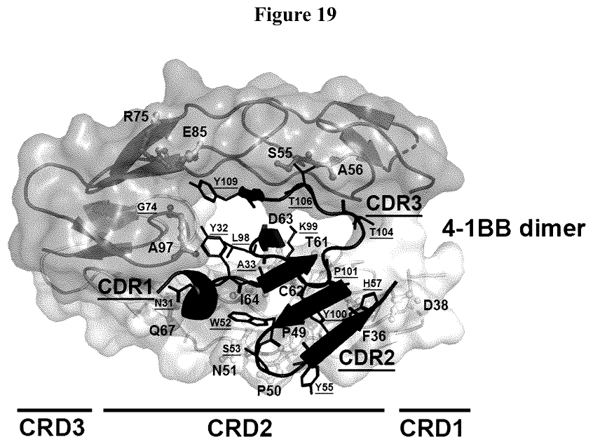

The VHH (BGA-2524) in complex with CD137 crystallized in the P2₁ space group, with two complex in the asymmetric unit, and diffracted to 1.71 Å. The structure of VHH (BGA-2524) bound to human CD137 shows that VHH (BGA-2524) partially sterically interfaces with CD137L binding (FIG. 17). The buried surface area between VHH (BGA-2524) and CD137 is approximately 863 Å². VHH (BGA-2524) interactions are clustered around CD137 CRD2 domain. VHH (BGA-2524) mainly binds the side surface of CD137 CRD2 domain with CDR residues. All CDRs of VHH (BGA-2524) participate in CD137 dimer binding, and especially CDR3 contributes mostpotential. CDR1 and CDR3 binds both monomers of CD137 dimer while CDR2 only binds one monomer of CD137 dimer. The bended CDR3 loop always covers hydrophobic patch of VHH framework, which could result in better biophysical property (FIG. 18). VHH (BGA-2524) CDR1 Tyr32 contact one monomer of CD137 dimer at residues Gly98, meanwhile CDR1 Asn31 and Ala33 contact another monomer of CD137 dimer at residues Ile64 and Gln67. VHH (BGA-2524) CDR2 Trp52, Ser53, Tyr55, His57 only contact one monomer of CD137 dimer at residues Asp38, Pro49, Pro50, Asn51, Ile64. VHH (BGA-2524) CDR3 residues Leu98, Thr104, Thr106 and Tyr109 contact one monomer of CD137 dimer at residues Ser55, Ala56, Arg75, Glu85 and Ala97, meanwhile CDR3 residues Leu98, Lys99, Tyr100 and Pro101 contact another monomer of CD137 dimer at residues Phe36, Pro49, Thr61, Cys62, Asp63 and Ile64. VHH (BGA-2524) interacts with CD137 using a combination of hydrogen bonds and salt bridges together with hydrophobic interactions. For example, VHH (BGA-2524) CDR2 residue His57 forms two salt bridges with CD137 residues Asp38. VHH (BGA-2524) CDR3 residue Lys99 forms two salt bridges with CD137 residue Asp63. VHH (BGA-2524) residues Tyr32, Ser53, His57, Leu98, Lys99, Pro101 and Thr106 form one hydrogen bond with CD137 residues Gly98, Asn51, Asp38, Ile64, Asp63, Thr61 and Ser55, respectively (i.e., Tyr32-Gly98, Ser53-Asn51, His57-Asp38, Leu98-Ile64, Lys99-Asp63, Pro101-Thr61 and Thr106-Ser55). VHH (BGA-2524) residue Trp52 forms two hydrogen bonds with CD137 residues Pro50 and Asn51. VHH (BGA-2524) residue Tyr109 forms two hydrogen bonds with CD137 residues Arg75 and Glu85. VHH (BGA-2524) residue Tyr100 forms two hydrogen bonds with CD137 residue Cys62 (FIG. 19).

Based on the crystal structure of the VHH (BGA-2524)/CD137 complex, the residues of CD137 that are contacted by VHH (BGA-2524) (i.e., the epitopic residues of CD137 bound by VHH (BGA-2524)) and the residues of VHH (BGA-2524) that are contacted by CD137 (i.e. the paratopic residues of VHH (BGA-2524) contacted by CD137) were determined. Table 19 and Table 20, below, show the residues of CD137 and VHH (BGA-2524) to which they contact, as assessed using a contact distance stringency of 3.7 Å, a point at which van der Waals (non-polar) interaction forces are highest.

TABLE 19
Epitopic residues of CD137 and their corresponding
paratopic residues of VHH (BGA-2524)

| | | | | | Natural | Numbered in |
|---|---|---|---|---|---|---|
| CD137 | | CD137 | | VHH | order of | Kabat |
| monomer | | monomer | | (BGA- | sequence | nomenclature |
| 1 | | 2 | | 2524) | for VHH | for VHH |
| | | PHE | 36 | PRO | 101 | H99 |
| | | ASP | 38 | HIS | 57 | H58 |
| | | PRO | 49 | TRP | 52 | H52 |
| | | | | TYR | 55 | H56 |
| | | | | TYR | 100 | H98 |
| | | PRO | 50 | TRP | 52 | H52 |
| | | | | SER | 53 | H54 |
| | | | | TYR | 55 | H56 |
| | | ASN | 51 | TRP | 52 | H52 |
| | | | | SER | 53 | H54 |
| SER | 55 | | | THR | 106 | H100D |
| ALA | 56 | | | THR | 104 | H100B |
| | | | | THR | 106 | H100D |
| | | THR | 61 | LYS | 99 | H97 |
| | | | | TYR | 100 | H98 |
| | | | | PRO | 101 | H99 |
| | | CYS | 62 | LYS | 99 | H97 |
| | | | | TYR | 100 | H98 |
| | | ASP | 63 | LEU | 98 | H96 |
| | | | | LYS | 99 | H97 |
| | | ILE | 64 | ALA | 33 | H33 |
| | | | | TRP | 52 | H52 |
| | | | | LEU | 98 | H96 |
| | | GLN | 67 | ASN | 31 | H31 |
| ARG | 75 | | | TYR | 109 | H102 |
| GLU | 85 | | | TYR | 109 | H102 |
| ALA | 97 | | | TYR | 32 | H32 |
| | | | | LEU | 98 | H96 |
| GLY | 98 | | | TYR | 32 | H32 |

TABLE 20
Paratopic residues of VHH (BGA-2524) and their
corresponding epitopic residues of CD137

| | | | | | | Numbered |
|---|---|---|---|---|---|---|
| | Natural | | | | | in Kabat |
| VHH | order of | CD137 | | CD137 | Natural | nomen- |
| (BGA- | sequence | monomer | | monomer | order of | clature |
| 2524) | for VHH | 1 | | 2 | sequence | for VHH |
| ASN | 31 | | | GLN | 67 | H31 |
| TYR | 32 | ALA | 97 | | | H32 |
| | | GLY | 98 | | | |
| ALA | 33 | | | ILE | 64 | H33 |
| TRP | 52 | | | PRO | 49 | H52 |
| | | | | PRO | 50 | |
| | | | | ASN | 51 | |
| | | | | ILE | 64 | |
| SER | 53 | | | PRO | 50 | H54 |
| | | | | ASN | 51 | |
| TYR | 55 | | | PRO | 49 | H56 |
| | | | | PRO | 50 | |
| HIS | 57 | | | ASP | 38 | H58 |
| LEU | 98 | ALA | 97 | ASP | 63 | H96 |
| | | | | ILE | 64 | |
| LYS | 99 | | | THR | 61 | H97 |
| | | | | CYS | 62 | |
| | | | | ASP | 63 | |
| TYR | 100 | | | PRO | 49 | H98 |
| | | | | THR | 61 | |
| | | | | CYS | 62 | |
| PRO | 101 | | | PHE | 36 | H99 |
| | | | | THR | 61 | |
| THR | 104 | ALA | 56 | | | H100B |
| THR | 106 | SER | 55 | | | H100D |
| | | ALA | 56 | | | |
| TYR | 109 | ARG | 75 | | | H102 |
| | | GLU | 85 | | | |

1-33. (canceled)

34. A multispecific antibody or antigen-binding fragment thereof, comprising a first antigen binding domain that specifically binds to human Glypican 3 (GPC3) and a second antigen binding domain that specifically binds to human CD137, wherein the first antigen binding domain that specifically binds to human GPC3 comprises
a heavy chain variable region (VH) that comprises (a) a HCDR1 of SEQ ID NO: 45, (b) a HCDR2 of SEQ ID NO: 46, and (c) a HCDR3 of SEQ ID NO: 47; and
a light chain variable region (VL) that comprises (d) a LCDR1 of SEQ ID NO: 48, (e) a LCDR2 of SEQ ID NO: 49, and (f) a LCDR3 of SEQ ID NO: 50, and
wherein the second antigen binding domain that specifically binds to human CD137 comprises:
(i) a heavy chain variable region (VH) that comprises (a) a HCDR1 of SEQ ID NO: 1, (b) a HCDR2 of SEQ ID NO: 2, and (c) a HCDR3 of SEQ ID NO: 3; or
(ii) a heavy chain variable region (VH) that comprises (a) a HCDR1 of SEQ ID NO: 1, (b) a HCDR2 of SEQ ID NO: 10, and (c) a HCDR3 of SEQ ID NO: 3.

35. The multispecific antibody or antigen-binding fragment thereof of claim 34, wherein the first antigen binding domain that specifically binds to human GPC3 comprises:
a heavy chain variable region (VH) that comprises the amino acid sequence at least 95% identical to SEQ ID NO: 41, and a light chain variable region (VL) that comprises the amino acid sequence at least 95% identical to SEQ ID NO: 43; and
wherein the second antigen binding domain that specifically binds to human CD137 comprises:
(i) a heavy chain variable region (VH) that comprises the amino acid sequence at least 95% identical SEQ ID NO: 17;
(ii) a heavy chain variable region (VH) that comprises the amino acid sequence at least 95% identical SEQ ID NO: 4;
(iii) a heavy chain variable region (VH) that comprises the amino acid sequence at least 95% identical SEQ ID NO: 11;
(iv) a heavy chain variable region (VH) that comprises the amino acid sequence at least 95% identical SEQ ID NO: 13; or
(v) a heavy chain variable region (VH) that comprises the amino acid sequence at least 95% identical SEQ ID NO: 15.

36. The multispecific antibody or antigen-binding fragment thereof of claim 34, wherein the first antigen binding domain that specifically binds to human GPC3 comprises:
a heavy chain variable region (VH) that comprises the amino acid sequence of SEQ ID NO: 41, and a light chain variable region (VL) that comprises the amino acid sequence of SEQ ID NO: 43; and
wherein the second antigen binding domain that specifically binds to human CD137 comprises:
a heavy chain variable region (VH) that comprises the amino acid sequence of SEQ ID NO: 17.

37. (canceled)

38. The multispecific antibody or antigen-binding fragment thereof of claim 34, wherein the multispecific antibody or antigen-binding fragment thereof is a bispecific antibody.

39-42. (canceled)

43. The multispecific antibody or antigen-binding fragment thereof of claim 34, wherein the multispecific antibody or antigen-binding fragment comprises a heavy chain constant region of the subclass of IgG1, and a light chain constant region of the type of kappa.

44-47. (canceled)

48. The multispecific antibody or antigen-binding fragment thereof of claim 34, wherein the multispecific antibody or antigen-binding fragment thereof comprises a Fc domain, and wherein the Fc domain comprises the amino acid sequence of SEQ ID NO: 20.

49-50. (canceled)

51. The multispecific antibody or antigen-binding fragment thereof of claim 34, wherein the multispecific antibody or antigen-binding fragment comprises
a first polypeptide of SEQ ID NO: 21 and a second polypeptide of SEQ ID NO: 23.

52. (canceled)

53. A method of treating a cancer comprising administering to a patient in need thereof a therapeutically effective amount of the multispecific antibody or antigen-binding fragment thereof of claim 34.

54-56. (canceled)

57. The method of claim 53, wherein the liver cancer is hepatoblastoma or hepatocellular carcinoma (HCC).

58-72. (canceled)

73. A multispecific antibody or antigen-binding fragment thereof, comprising a first polypeptide of SEQ ID NO: 21 and a second polypeptide of SEQ ID NO: 23.

74. A pharmaceutical composition comprising the multispecific antibody or antigen-binding fragment thereof of claim 73 and a pharmaceutically acceptable carrier.

75. An isolated nucleic acid that encodes the multispecific antibody or antigen-binding fragment thereof of claim 73.

76. A vector comprising the nucleic acid of claim 75.

77. A host cell comprising the nucleic acid of claim 75.

78. A process for producing a multispecific antibody or antigen-binding fragment thereof comprising cultivating the host cell of claim 77 and recovering the antibody or antigen-binding fragment thereof from the culture.

79. An antibody or antigen-binding fragment thereof which specifically binds human CD137, comprising:
(i) a heavy chain variable region (VH) that comprises (a) a HCDR1 (Heavy Chain Complementarity Determining Region 1) of SEQ ID NO: 1, (b) a HCDR2 of SEQ ID NO: 2, and (c) a HCDR3 of SEQ ID NO: 3; or
(ii) a heavy chain variable region that comprises (a) a HCDR1 of SEQ ID NO:1, (b) a HCDR2 of SEQ ID NO: 10, and (c) a HCDR3 of SEQ ID NO: 3.

80. A multispecific antibody or antigen-binding fragment thereof, comprising at least a first antigen binding domain that specifically binds a human tumor-associated antigen (TAA), and at least a second antigen binding domain that specifically binds human CD137, wherein the second antigen binding domain comprises:
(i) a heavy chain variable region (VH) that comprises (a) a HCDR1 of SEQ ID NO: 1, (b) a HCDR2 of SEQ ID NO: 2, and (c) a HCDR3 of SEQ ID NO: 3; or
(ii) a heavy chain variable region (VH) that comprises (a) a HCDR1 of SEQ ID NO: 1, (b) a HCDR2 of SEQ ID NO: 10, and (c) a HCDR3 of SEQ ID NO: 3.


SEQUENCE LISTING (105 sequences)

SEQ ID NO: 1 - protein, 5 aa, synthetic construct
  source 1..5: note = HCDR1 (Kabat)
NYAVG

SEQ ID NO: 2 - protein, 15 aa, synthetic construct
  source 1..15: note = HCDR2 (Kabat)
TYWSGYAHYGDFENN

SEQ ID NO: 3 - protein, 13 aa, synthetic construct
  source 1..13: note = HCDR3 (Kabat)
SLKYPMATVTPDY

SEQ ID NO: 4 - protein, 120 aa, synthetic construct
  source 1..120: note = VH AA
QVQLVESGGGTVQPGDSLRLSCAASGRTFDNYAVGWFRQAPGKERQYVGTYWSGYAHYGDFENNRFTISRDNAKNTVYLQMDNLKPEDTAVYYCAISLKYPMATVTPDYQGQGTQVTVSS

SEQ ID NO: 5 - DNA, 360 bp, synthetic construct
  source 1..360: mol_type = other DNA; note = VH DNA
caggtgcagctcgtggagtccgggggaggaacggtgcagcctggggactctctgagactctcctgtgctgcctctggacgcaccttcgataactatgccgtgggctggttccgccaggctccagggaaggagcgtcagtatgtcggcacgtattggagtggttacgcacactatggggactttgagaataaccgattcacgatctccagagacaacgccaagaatacggtgtatctgcaaatggacaacctgaaacctgaggacacggccgtttattactgtgcaatatcccttaaatatcctatggcgacagttaccccagactaccagggccaggggacccaggtcaccgtctcctca

SEQ ID NO: 6 - protein, 120 aa, synthetic construct
  source 1..120: note = VH AA
EVQLLESGGGLVQPGGSLRLSCAASGRTFDNYAVGWFRQAPGKEREYVGTYWSGYAHYGDFENNRFTISRDNSKNTVYLQMNSLRAEDTAVYYCAISLKYPMATVTPDYQGQGTLVTVSS

SEQ ID NO: 7 - DNA, 360 bp, synthetic construct
  source 1..360: mol_type = other DNA; note = VH DNA
gaagtgcagctgctggagtccgggggcggcctggtccaacccgggggcagcctgagactgagctgcgccgcctccggcagaaccttcgacaactacgccgtgggctggttcagacaagcccccggcaaggagagagagtacgtgggcacctactggagcggctacgcccactacggcgacttcgagaacaacagattcaccatcagcagagacaacagcaagaacaccgtgtacctgcagatgaacagcctgagagccgaggacaccgccgtgtactactgcgccatcagcctgaagtaccccatggccaccgtgacccccgactaccaaggccaaggcaccctggtgaccgtgagcagc

SEQ ID NO: 8 - protein, 120 aa, synthetic construct
  source 1..120: note = VHH AA
EVQLLESGGGLVQPGGSLRLSCAASGRTFDNYAVGWFRQAPGKERQYVGTYWSGYAHYGDFENNRFTISRDNSKNTVYLQMNSLRAEDTAVYYCAISLKYPMATVTPDYQGQGTLVTVSS

SEQ ID NO: 9 - DNA, 360 bp, synthetic construct
  source 1..360: mol_type = other DNA; note = VHH DNA
gaagtgcagctgctggagtccgggggcggcctggtccaacccgggggcagcctgagactgagctgcgccgcctccggcagaaccttcgacaactacgccgtgggctggttcagacaagcccccggcaaggagagacagtacgtgggcacctactggagcggctacgcccactacggcgacttcgagaacaacagattcaccatcagcagagacaacagcaagaacaccgtgtacctgcagatgaacagcctgagagccgaggacaccgccgtgtactactgcgccatcagcctgaagtaccccatggccaccgtgacccccgactaccaaggccaaggcaccctggtgaccgtgagcagc

SEQ ID NO: 10 - protein, 15 aa, synthetic construct
  source 1..15: note = CDR2 (Kabat)
TYWSGYAHYGDFEKG

SEQ ID NO: 11 - protein, 120 aa, synthetic construct
  source 1..120: note = VHH AA
EVQLVESGGGLVQPGGSLRLSCAASGRTFDNYAVGWFRQAPGKEREYVGTYWSGYAHYGDFEKGRFTISRDNSKNTVYLQMNNLRPEDTAVYYCAISLKYPMATVTPDYQGQGTQVTVSS

SEQ ID NO: 12 - DNA, 360 bp, synthetic construct
  source 1..360: mol_type = other DNA; note = VHH DNA
gaagtgcagctggtggagtccgggggcggcctggtccaacccgggggcagcctgagactgagctgcgccgcctccggcagaaccttcgacaactacgccgtgggctggttcagacaagcccccggcaaggagagagagtacgtgggcacctactggagcggctacgcccactacggcgacttcgagaagggcagattcaccatcagcagagacaacagcaagaacaccgtgtacctgcagatgaacaacctgagacccgaggacaccgccgtgtactactgcgccatcagcctgaagtaccccatggccaccgtgacccccgactaccaaggccaaggcacccaagtgaccgtgagcagc

SEQ ID NO: 13 - protein, 120 aa, synthetic construct
  source 1..120: note = VHH AA
EVQLVESGGGLVQPGGSLRLSCAASGRTFDNYAVGWFRQAPGKEREYVGTYWSGYAHYGDFEKGRFTISRDNAKNTVYLQMNNLRPEDTAVYYCAISLKYPMATVTPDYQGQGTQVTVSS

SEQ ID NO: 14 - DNA, 360 bp, synthetic construct
  source 1..360: mol_type = other DNA; note = VHH DNA
gaagtgcagctggtggagtccgggggcggcctggtccaacccgggggcagcctgagactgagctgcgccgcctccggcagaaccttcgacaactacgccgtgggctggttcagacaagcccccggcaaggagagagagtacgtgggcacctactggagcggctacgcccactacggcgacttcgagaagggcagattcaccatcagcagagacaacgccaagaacaccgtgtacctgcagatgaacaacctgagacccgaggacaccgccgtgtactactgcgccatcagcctgaagtaccccatggccaccgtgacccccgactaccaaggccaaggcacccaagtgaccgtgagcagc

SEQ ID NO: 15 - protein, 120 aa, synthetic construct
  source 1..120: note = VHH AA
EVQLVESGGGLVQPGGSLRLSCAASGRTFDNYAVGWFRQAPGKEREYVGTYWSGYAHYGDFENNRFTISRDNSKNTVYLQMNNLRPEDTAVYYCAISLKYPMATVTPDYQGQGTQVTVSS

SEQ ID NO: 16 - DNA, 360 bp, synthetic construct
  source 1..360: mol_type = other DNA; note = VHH DNA
gaagtgcagctggtggagtccgggggcggcctggtccaacccgggggcagcctgagactgagctgcgccgcctccggcagaaccttcgacaactacgccgtgggctggttcagacaagcccccggcaaggagagagagtacgtgggcacctactggagcggctacgcccactacggcgacttcgagaacaacagattcaccatcagcagagacaacagcaagaacaccgtgtacctgcagatgaacaacctgagacccgaggacaccgccgtgtactactgcgccatcagcctgaagtaccccatggccaccgtgacccccgactaccaaggccaaggcacccaagtgaccgtgagcagc

SEQ ID NO: 17 - protein, 120 aa, synthetic construct
  source 1..120: note = VHH AA
EVQLVESGGGLVQPGGSLRLSCAASGRTFDNYAVGWFRQAPGKEREYVGTYWSGYAHYGDFENNRFTISRDNAKNTVYLQMNNLRPEDTAVYYCAISLKYPMATVTPDYQGQGTQVTVSS

SEQ ID NO: 18 - DNA, 360 bp, synthetic construct
  source 1..360: mol_type = other DNA; note = VHH DNA
gaagtgcagctggtggagtccgggggcggcctggtccaacccgggggcagcctgagactgagctgcgccgctagcggcagaaccttcgacaactacgccgtgggctggttcagacaagcccccggcaaggagagagagtacgtgggcacctactggagcggctacgcccactacggcgacttcgagaacaacagattcaccatcagcagagacaacgccaagaacaccgtgtacctgcagatgaacaacctgagacccgaggacaccgccgtgtactactgcgccatcagcctgaagtaccccatggccaccgtgacccccgactaccaaggccaaggcacccaagtgaccgtgagcagc

SEQ ID NO: 19 - protein, 231 aa, Homo sapiens
  source 1..231: note = Fc of human IgG1 mf AA
EPKSSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 231 aa, Homo sapiens
  source 1..231: note = Fc of human IgG1 mf AA with YTE mutation
EPKSCDKTHTCPPCPAPPAAGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 569 aa, synthetic construct
  source 1..569: note = VH
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGLVQPGGSLRLSCAASGRTFDNYAVGWFRQAPGKEREYVGTYWSGYAHYGDFENNRFTISRDNAKNTVYLQMNNLRPEDTAVYYCAISLKYPMATVTPDYQGQGTQVTVSS

SEQ ID NO: 22 - DNA, 1707 bp, synthetic construct
  source 1..1707: mol_type = other DNA; note = VH DNA
caagtgcagctggtgcagagcggcgccgaggtgaagaagcccggcgctagcgtgaaggtgagctgcaaggctagcggctacaccttcaccgactacgaaatgcactgggtcagacaagcccccggccaaggcctggaatggatgggcgccctggaccccaagaccggcgacaccgcctacagccaaaagttcaagggcagagtgaccctgaccgccgacaagagcacaagcaccgcctacatggagctgagcagcctgacaagcgaggacaccgccgtctactactgcacaagattctacagctacacctactggggccaaggcaccctcgtgaccgtgagctccgctagcacaaagggccctagcgtgttccccctcgctcctagcagcaagagcacctccgggggcacagccgccctgggctgcctggtgaaggactacttccccgagcccgtgaccgtgagctggaacagcggcgccctcacaagcggcgtgcataccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcccaagtcctgtgataagacccacacatgccctccctgccccgcccctcccgccgccggcccttccgtgttcctgttcccccccaagcccaaggacaccctgtacatcaccagagagcccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctggccgcccccatcgagaagaccatcagcaaggccaaggggcagcctagagagccccaagtgtacaccctgccccctagcagagaggagatgaccaagaaccaagtgagcctgacctgcctcgtgaagggcttctaccctagcgacatcgccgtggagtgggagagcaacgggcagcccgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacccaaaaaagcctcagcctcagccccggcaagggcgggggcggctccgaagtgcagctggtggagtccgggggcggcctggtccaacccgggggcagcctgagactgagctgcgccgctagcggcagaaccttcgacaactacgccgtgggctggttcagacaagcccccggcaaggagagagagtacgtgggcacctactggagcggctacgcccactacggcgacttcgagaacaacagattcaccatcagcagagacaacgccaagaacaccgtgtacctgcagatgaacaacctgagacccgaggacaccgccgtgtactactgcgccatcagcctgaagtaccccatggccaccgtgacccccgactaccaaggccaaggcacccaagtgaccgtgagcagc

SEQ ID NO: 23 - protein, 219 aa, synthetic construct
  source 1..219: note = VL AA
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 657 bp, synthetic construct
  source 1..657: mol_type = other DNA; note = VL DNA
gacgtggtgatgacacagagccccctgtccctgcctgtgacccccggggaacccgctagcatcagctgcagaagcagccaaagcctggtgcacagcaacagaaacacctacctgcactggtacctgcagaagcccgggcagagccctcagctgctgatctacaaggtgagcaacagattcagcggcgtgcccgacagattctccggcagcggcagcggcaccgacttcaccctgaagatcagcagagtggaggccgaggacgtgggcgtgtactactgcagccaaaacacccacgtgccccccaccttcggccaaggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 25 - protein, 569 aa, synthetic construct
  source 1..569: note = VH AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGTVQPGDSLRLSCAASGRTFDNYAVGWFRQAPGKERQYVGTYWSGYAHYGDFENNRFTISRDNAKNTVYLQMDNLKPEDTAVYYCAISLKYPMATVTPDYQGQGTQVTVSS

SEQ ID NO: 26 - DNA, 1707 bp, synthetic construct
  source 1..1707: mol_type = other DNA; note = VH DNA
caggtgcagctggtgcagtctggagcagaggtgaagaagccaggagccagcgtgaaggtgtcctgcaaggcctctggctataccttcacagactacgagatgcactgggtgcggcaggcaccaggacagggactggagtggatgggcgccctggaccctaagaccggcgatacagcctatagccagaagtttaagggcagagtgaccctgacagccgacaagtccacctctacagcctacatggagctgagctccctgaccagcgaggatacagccgtgtactattgtacccggttctactcctatacatactggggccagggcaccctggtgacagtgtctagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgtacatcacccgggagccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtgcagctcgtggagtccgggggaggaacggtgcagcctggggactctctgagactctcctgtgctgcctctggacgcaccttcgataactatgccgtgggctggttccgccaggctccagggaaggagcgtcagtatgtcggcacgtattggagtggttacgcacactatggggactttgagaataaccgattcacgatctccagagacaacgccaagaatacggtgtatctgcaaatggacaacctgaaacctgaggacacggccgtttattactgtgcaatatcccttaaatatcctatggcgacagttaccccagactaccagggccaggggacccaggtcaccgtctcctca

SEQ ID NO: 27 - protein, 569 aa, synthetic construct
  source 1..569: note = VH AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGTVQPGDSLRLSCAASGRTFDNYAVGWFRQAPGKERQYVGTYWSGYAHYGDFENNRFTISRDNAKNTVYLQMDNLKPEDTAVYYCAISLKYPMATVTPDYQGQGTQVTVSS

SEQ ID NO: 28 - DNA, 1707 bp, synthetic construct
  source 1..1707: mol_type = other DNA; note = VH DNA
caggtgcagctggtgcagtctggagcagaggtgaagaagccaggagccagcgtgaaggtgtcctgcaaggcctctggctataccttcacagactacgagatgcactgggtgcggcaggcaccaggacagggactggagtggatgggcgccctggaccctaagaccggcgatacagcctatagccagaagtttaagggcagagtgaccctgacagccgacaagtccacctctacagcctacatggagctgagctccctgaccagcgaggatacagccgtgtactattgtacccggttctactcctatacatactggggccagggcaccctggtgacagtgtctagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtgcagctcgtggagtccgggggaggaacggtgcagcctggggactctctgagactctcctgtgctgcctctggacgcaccttcgataactatgccgtgggctggttccgccaggctccagggaaggagcgtcagtatgtcggcacgtattggagtggttacgcacactatggggactttgagaataaccgattcacgatctccagagacaacgccaagaatacggtgtatctgcaaatggacaacctgaaacctgaggacacggccgtttattactgtgcaatatcccttaaatatcctatggcgacagttaccccagactaccagggccaggggacccaggtcaccgtctcctca

SEQ ID NO: 29 - protein, 569 aa, synthetic construct
  source 1..569: note = VH AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGLVQPGGSLRLSCAASGRTFDNYAVGWFRQAPGKEREYVGTYWSGYAHYGDFENNRFTISRDNSKNTVYLQMNNLRPEDTAVYYCAISLKYPMATVTPDYQGQGTQVTVSS

SEQ ID NO: 30 - DNA, 1710 bp, synthetic construct
  source 1..1710: mol_type = other DNA; note = VH DNA
caagtgcagctggtgcagagcggcgccgaggtgaagaagcccggcgctagcgtgaaggtgagctgcaaggctagcggctacaccttcaccgactacgaaatgcactgggtcagacaagcccccggccaaggcctggaatggatgggcgccctggaccccaagaccggcgacaccgcctacagccaaaagttcaagggcagagtgaccctgaccgccgacaagagcacaagcaccgcctacatggagctgagcagcctgacaagcgaggacaccgccgtctactactgcacaagattctacagctacacctactggggccaaggcaccctcgtgaccgtgagctccgctagcacaaagggccctagcgtgttccccctcgctcctagcagcaagagcacctccgggggcacagccgccctgggctgcctggtgaaggactacttccccgagcccgtgaccgtgagctggaacagcggcgccctcacaagcggcgtgcataccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcccaagtcctgtgataagacccacacatgccctccctgccccgcccctcccgccgccggcccttccgtgttcctgttcccccccaagcccaaggacaccctgatgatcagcagaacccccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctggccgcccccatcgagaagaccatcagcaaggccaaggggcagcctagagagccccaagtgtacaccctgccccctagcagagaggagatgaccaagaaccaagtgagcctgacctgcctcgtgaagggcttctaccctagcgacatcgccgtggagtgggagagcaacgggcagcccgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacccaaaaaagcctcagcctcagccccggcaagggcgggggcggctccgaagtgcagctggtggagtccgggggcggcctggtccaacccgggggcagcctgagactgagctgcgccgctagcggcagaaccttcgacaactacgccgtgggctggttcagacaagcccccggcaaggagagagagtacgtgggcacctactggagcggctacgcccactacggcgacttcgagaacaacagattcaccatcagcagagacaacagcaagaacaccgtgtacctgcagatgaacaacctgagacccgaggacaccgccgtgtactactgcgccatcagcctgaagtaccccatggccaccgtgacccccgactaccaaggccaaggcacccaagtgaccgtgagcagctga

SEQ ID NO: 31 - protein, 569 aa, synthetic construct
  source 1..569: note = VH AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGLVQPGGSLRLSCAASGRTFDNYAVGWFRQAPGKEREYVGTYWSGYAHYGDFEKGRFTISRDNAKNTVYLQMNNLRPEDTAVYYCAISLKYPMATVTPDYQGQGTQVTVSS

SEQ ID NO: 32 - DNA, 1710 bp, synthetic construct
  source 1..1710: mol_type = other DNA; note = VH DNA
caagtgcagctggtgcagagcggcgccgaggtgaagaagcccggcgctagcgtgaaggtgagctgcaaggctagcggctacaccttcaccgactacgaaatgcactgggtcagacaagcccccggccaaggcctggaatggatgggcgccctggaccccaagaccggcgacaccgcctacagccaaaagttcaagggcagagtgaccctgaccgccgacaagagcacaagcaccgcctacatggagctgagcagcctgacaagcgaggacaccgccgtctactactgcacaagattctacagctacacctactggggccaaggcaccctcgtgaccgtgagctccgctagcacaaagggccctagcgtgttccccctcgctcctagcagcaagagcacctccgggggcacagccgccctgggctgcctggtgaaggactacttccccgagcccgtgaccgtgagctggaacagcggcgccctcacaagcggcgtgcataccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcccaagtcctgtgataagacccacacatgccctccctgccccgcccctcccgccgccggcccttccgtgttcctgttcccccccaagcccaaggacaccctgatgatcagcagaacccccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctggccgcccccatcgagaagaccatcagcaaggccaaggggcagcctagagagccccaagtgtacaccctgccccctagcagagaggagatgaccaagaaccaagtgagcctgacctgcctcgtgaagggcttctaccctagcgacatcgccgtggagtgggagagcaacgggcagcccgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacccaaaaaagcctcagcctcagccccggcaagggcgggggcggctccgaagtgcagctggtggagtccgggggcggcctggtccaacccgggggcagcctgagactgagctgcgccgctagcggcagaaccttcgacaactacgccgtgggctggttcagacaagcccccggcaaggagagagagtacgtgggcacctactggagcggctacgcccactacggcgacttcgagaaggggagattcaccatcagcagagacaacgccaagaacaccgtgtacctgcagatgaacaacctgagacccgaggacaccgccgtgtactactgcgccatcagcctgaagtaccccatggccaccgtgacccccgactaccaaggccaaggcacccaagtgaccgtgagcagctga

SEQ ID NO: 33 - protein, 569 aa, synthetic construct
  source 1..569: note = VH AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGLVQPGGSLRLSCAASGRTFDNYAVGWFRQAPGKEREYVGTYWSGYAHYGDFEKGRFTISRDNAKNTVYLQMNNLRPEDTAVYYCAISLKYPMATVTPDYQGQGTQVTVSS

SEQ ID NO: 34 - DNA, 1707 bp, synthetic construct
  source 1..1707: mol_type = other DNA; note = VH DNA
caagtgcagctggtgcagagcggcgccgaggtgaagaagcccggcgctagcgtgaaggtgagctgcaaggctagcggctacaccttcaccgactacgaaatgcactgggtcagacaagcccccggccaaggcctggaatggatgggcgccctggaccccaagaccggcgacaccgcctacagccaaaagttcaagggcagagtgaccctgaccgccgacaagagcacaagcaccgcctacatggagctgagcagcctgacaagcgaggacaccgccgtctactactgcacaagattctacagctacacctactggggccaaggcaccctcgtgaccgtgagctccgctagcacaaagggccctagcgtgttccccctcgctcctagcagcaagagcacctccgggggcacagccgccctgggctgcctggtgaaggactacttccccgagcccgtgaccgtgagctggaacagcggcgccctcacaagcggcgtgcataccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcccaagtcctgtgataagacccacacatgccctccctgccccgcccctcccgccgccggcccttccgtgttcctgttcccccccaagcccaaggacaccctgtacatcaccagagagcccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctggccgcccccatcgagaagaccatcagcaaggccaaggggcagcctagagagccccaagtgtacaccctgccccctagcagagaggagatgaccaagaaccaagtgagcctgacctgcctcgtgaagggcttctaccctagcgacatcgccgtggagtgggagagcaacgggcagcccgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacccaaaaaagcctcagcctcagccccggcaagggcgggggcggctccgaagtgcagctggtggagtccgggggcggcctggtccaacccgggggcagcctgagactgagctgcgccgctagcggcagaaccttcgacaactacgccgtgggctggttcagacaagcccccggcaaggagagagagtacgtgggcacctactggagcggctacgcccactacggcgacttcgagaaggggagattcaccatcagcagagacaacgccaagaacaccgtgtacctgcagatgaacaacctgagacccgaggacaccgccgtgtactactgcgccatcagcctgaagtaccccatggccaccgtgacccccgactaccaaggccaaggcacccaagtgaccgtgagcagc

SEQ ID NO: 35 - protein, 255 aa, Homo sapiens
  source 1..255: note = Human CD137-full length AA
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 36 - DNA, 765 bp, Homo sapiens
  source 1..765: mol_type = other DNA; note = Human CD137-full length DNA on GenBank sequence Accession No: NM_001561.4
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 37 - protein, 254 aa, Homo sapiens
  source 1..254: note = Human CD137-ligand full length AA
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 38 - DNA, 765 bp, Homo sapiens
  source 1..765: mol_type = other DNA; note = Human CD137-ligand full length DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 39 - protein, 233 aa, synthetic construct
  source 1..233: note = mIgG2a AA
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 40 - DNA, 699 bp, synthetic construct
  source 1..699: mol_type = other DNA; note = mIgG2a DNA
gagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 41 - protein, 115 aa, synthetic construct
  source 1..115: note = GPC3 VH AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 42 - DNA, 345 bp, synthetic construct
  source 1..345: mol_type = other DNA; note = GPC3 VH DNA
caggtgcagctggtgcagtctggagcagaggtgaagaagccaggagccagcgtgaaggtgtcctgcaaggcctctggctataccttcacagactacgagatgcactgggtgcggcaggcaccaggacagggactggagtggatgggcgccctggaccctaagaccggcgatacagcctatagccagaagtttaagggcagagtgaccctgacagccgacaagtccacctctacagcctacatggagctgagctccctgaccagcgaggatacagccgtgtactattgtacccggttctactcctatacatactggggccagggcaccctggtgacagtgtctagc

SEQ ID NO: 43 - protein, 112 aa, synthetic construct
  source 1..112: note = GPC3 VL AA
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 44 - DNA, 336 bp, synthetic construct
  source 1..336: mol_type = other DNA; note = GPC3 VL DNA
gacgtggtcatgacacagtcccctctgtctctgccagtgaccccaggagagcctgcctctatcagctgcaggagctcccagagcctggtgcactccaaccgcaatacatacctgcactggtatctgcagaagccaggccagtccccccagctgctgatctacaaggtgtctaaccggttcagcggcgtgcccgacagattttccggctctggcagcggcaccgatttcacactgaagatctcccgggtggaggcagaggatgtgggcgtgtactattgttctcagaatacccacgtgccccctacatttggccagggcaccaagctggagatcaag

SEQ ID NO: 45 - protein, 5 aa, synthetic construct
  source 1..5: note = Anti-GPC3 antibody HCDR1 (Kabat)
DYEMH

SEQ ID NO: 46 - protein, 17 aa, synthetic construct
  source 1..17: note = Anti-GPC3 antibody HCDR2 (Kabat)
ALDPKTGDTAYSQKFKG

SEQ ID NO: 47 - protein, 6 aa, synthetic construct
  source 1..6: note = Anti-GPC3 antibody HCDR3 (Kabat)
FYSYTY

SEQ ID NO: 48 - protein, 16 aa, synthetic construct
  source 1..16: note = Anti-GPC3 antibody LCDR1 (Kabat)
RSSQSLVHSNRNTYLH

SEQ ID NO: 49 - protein, 7 aa, synthetic construct
  source 1..7: note = Anti-GPC3 antibody LCDR2 (Kabat)
KVSNRFS

SEQ ID NO: 50 - protein, 9 aa, synthetic construct
  source 1..9: note = Anti-GPC3 antibody LCDR3 (Kabat)
SQNTHVPPT

SEQ ID NO: 51 - protein, 580 aa, Homo sapiens
  source 1..580: note = Human GPC3 full length AA
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 52 - DNA, 1743 bp, Homo sapiens
  source 1..1743: mol_type = other DNA; note = Human GPC3 full length DNA
atggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtatgtccagaagaatgcaggaaagctgaccaccactattggcaagttatgtgcccattctcaacaacgccaatatagatctgcttattatcctgaagatctctttattgacaagaaagtattaaaagttgctcatgtagaacatgaagaaaccttatccagccgaagaagggaactaattcagaagttgaagtctttcatcagcttctatagtgctttgcctggctacatctgcagccatagccctgtggcggaaaacgacaccctttgctggaatggacaagaactcgtggagagatacagccaaaaggcagcaaggaatggaatgaaaaaccagttcaatctccatgagctgaaaatgaagggccctgagccagtggtcagtcaaattattgacaaactgaagcacattaaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacctcgggaacgttcattccccgctgaagcttctcaccagcatggccatctcggtggtgtgcttcttcttcctggtgcactga

SEQ ID NO: 53 - protein, 231 aa, synthetic construct
  source 1..231: note = IgG1mf Fc
EPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 8 aa, synthetic construct
  source 1..8: note = HCDR1 (IMGT)
GRTFDNYA

SEQ ID NO: 55 - protein, 6 aa, synthetic construct
  source 1..6: note = HCDR2 (IMGT)
YWSGYA

SEQ ID NO: 56 - protein, 15 aa, synthetic construct
  source 1..15: note = HCDR3 (IMGT)
AISLKYPMATVTPDY

SEQ ID NO: 57 - protein, 7 aa, synthetic construct
  source 1..7: note = HCDR1 (Chothia)
GRTFDNY

SEQ ID NO: 58 - protein, 4 aa, synthetic construct
  source 1..4: note = HCDR2 (Chothia)
WSGY

SEQ ID NO: 59 - protein, 13 aa, synthetic construct
  source 1..13: note = HCDR3 (Chothia)
SLKYPMATVTPDY

SEQ ID NO: 60 - protein, 4 aa, synthetic construct
  source 1..4: note = linker
GGGS

SEQ ID NO: 61 - protein, 4 aa, synthetic construct
  source 1..4: note = SGGG
SGGG

SEQ ID NO: 62 - protein, 5 aa, synthetic construct
  source 1..5: note = linker
GGGGS

SEQ ID NO: 63 - protein, 7 aa, synthetic construct
  source 1..7: note = linker
GGGGSGS

SEQ ID NO: 64 - protein, 7 aa, synthetic construct
  source 1..7: note = linker
GGGGSGS

SEQ ID NO: 65 - protein, 8 aa, synthetic construct
  source 1..8: note = linker
GGGGSGGS

SEQ ID NO: 66 - protein, 10 aa, synthetic construct
  source 1..10: note = linker
GGGGSGGGGS

SEQ ID NO: 67 - protein, 15 aa, synthetic construct
  source 1..15: note = linker
GGGGSGGGGSGGGGS

SEQ ID NO: 68 - protein, 16 aa, synthetic construct
  source 1..16: note = linker
AKTTPKLEEGEFSEAR

SEQ ID NO: 69 - protein, 17 aa, synthetic construct
  source 1..17: note = linker
AKTTPKLEEGEFSEARV

SEQ ID NO: 70 - protein, 9 aa, synthetic construct
  source 1..9: note = linker
AKTTPKLGG

SEQ ID NO: 71 - protein, 10 aa, synthetic construct
  source 1..10: note = linker
SAKTTPKLGG

SEQ ID NO: 72 - protein, 17 aa, synthetic construct
  source 1..17: note = linker
AKTTPKLEEGEFSEARV

SEQ ID NO: 73 - protein, 6 aa, synthetic construct
  source 1..6: note = linker
SAKTTP

SEQ ID NO: 74 - protein, 10 aa, synthetic construct
  source 1..10: note = linker
SAKTTPKLGG

SEQ ID NO: 75 - protein, 6 aa, synthetic construct
  source 1..6: note = linker
RADAAP

SEQ ID NO: 76 - protein, 9 aa, synthetic construct
  source 1..9: note = linker
RADAAPTVS

SEQ ID NO: 77 - protein, 12 aa, synthetic construct
  source 1..12: note = linker
RADAAAAGGPGS

SEQ ID NO: 78 - protein, 27 aa, synthetic construct
  source 1..27: note = linker
RADAAAAGGGGSGGGGSGGGGSGGGGS

SEQ ID NO: 79 - protein, 6 aa, synthetic construct
  source 1..6: note = linker
SAKTTP

SEQ ID NO: 80 - protein, 10 aa, synthetic construct
  source 1..10: note = linker
SAKTTPKLGG

SEQ ID NO: 81 - protein, 18 aa, synthetic construct
  source 1..18: note = linker
SAKTTPKLEEGEFSEARV

SEQ ID NO: 82 - protein, 5 aa, synthetic construct
  source 1..5: note = linker
ADAAP

SEQ ID NO: 83 - protein, 12 aa, synthetic construct
  source 1..12: note = linker
ADAAPTVSIFPP

SEQ ID NO: 84 - protein, 5 aa, synthetic construct
  source 1..5: note = linker
TVAAP

SEQ ID NO: 85 - protein, 12 aa, synthetic construct
  source 1..12: note = linker
TVAAPSVFIFPP

SEQ ID NO: 86 - protein, 6 aa, synthetic construct
  source 1..6: note = linker
QPKAAP

SEQ ID NO: 87 - protein, 13 aa, synthetic construct
  source 1..13: note = linker
QPKAAPSVTLFPP

SEQ ID NO: 88 - protein, 6 aa, synthetic construct
  source 1..6: note = linker
AKTTPP

SEQ ID NO: 89 - protein, 13 aa, synthetic construct
  source 1..13: note = linker
AKTTPPSVTPLAP

SEQ ID NO: 90 - protein, 6 aa, synthetic construct
  source 1..6: note = linker
AKTTAP

SEQ ID NO: 91 - protein, 13 aa, synthetic construct
  source 1..13: note = linker
AKTTAPSVYPLAP

SEQ ID NO: 92 - protein, 6 aa, synthetic construct
  source 1..6: note = linker
ASTKGP

SEQ ID NO: 93 - protein, 13 aa, synthetic construct
  source 1..13: note = linker
ASTKGPSVFPLAP

SEQ ID NO: 94 - protein, 15 aa, synthetic construct
  source 1..15: note = linker
GENKVEYAPALMALS

SEQ ID NO: 95 - protein, 15 aa, synthetic construct
  source 1..15: note = linker
GPAKELTPLKEAKVS

SEQ ID NO: 96 - protein, 15 aa, synthetic construct
  source 1..15: note = linker
GHEAAAVMQVQYPAS

SEQ ID NO: 97-101 - skipped (5 entries)

SEQ ID NO: 102 - protein, 396 aa, synthetic construct
  source 1..396: note = Cyno CD137 ECD AA
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 103 - protein, 118 aa, synthetic construct
  source 1..118: note = Tislelizumab VH AA
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 104 - protein, 107 aa, synthetic construct
  source 1..107: note = Tislelizumab VH AA
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 105 - DNA, 1680 bp, Homo sapiens
  source 1..1680: mol_type = other DNA; note = Human CD137 ligand (TNFSF9) - CD137 ligand GenBank sequence (Accession No: NM_003811.3]
aaaaagcggcgcgctgtgtcttcccgcagtctctcgtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgtccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttgggggggggggggtcttcgacattgccgaggctggtcttgaactcctggacttagacgatcctcctgcctcagcctcccaagcaactgggattcatcctttctattaattcattgtacttatttgcttatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcctccctctgaaaaaaaaaaaaaaa